SEQUENCE LISTING (1 sequence)

SEQ ID NO: 1 - DNA, 20 bp, synthetic construct
  misc_feature 1..20: note = source = /note="Description of Artificial Sequence: Synthetic oligonucleotide"
  source 1..20: mol_type = other DNA
tgctccgttggtgcttgttc

(19) United States
(12) Patent Application Publication
(10) Pub. No.: US 2026/0049310 A1
(43) Pub. Date: Feb. 19, 2026

(54) METHOD FOR REDUCING THE RISK OF A CARDIOVASCULAR EVENT WITH CONJUGATED ANTISENSE COMPOUNDS TARGETING APO(A)

(71) Applicant: Novartis AG, Basel (CH)

(72) Inventors: Anastasia LESOGOR, Wenslingen (CH); Qing SHAO, Montville, NJ (US); Tom THUREN, East Hanover, NJ (US)

(21) Appl. No.: 19/171,501

(22) Filed: Apr. 7, 2025

Related U.S. Application Data
(63) Continuation of application No. 18/824,292, filed on Sep. 4, 2024, now abandoned.
Application No. 18/824,292 is a continuation of application No. 17/292,075, now abandoned.
(60) Provisional application No. 62/758,323, filed on Nov. 9, 2018.
(60) Provisional application No. 62/874,459, filed on Jul. 15, 2019.

(51) Int. Cl. C12N 15/113; A61P 9/00

(52) U.S. Cl. CPC C12N 15/113; A61P 9/00; C12N 2310/11; C12N 2310/341; C12N 2310/351; C12N 2320/30

(57) ABSTRACT

The present disclosure is directed to methods of reducing the risk of a cardiovascular event with conjugated antisense compounds targeting apo(a). Specifically, a method of reducing the risk of a cardiovascular event in a patient who has established cardiovascular disease with conjugated antisense compound ISIS 681257 or a salt thereof.

### RELATED APPLICATIONS

This application is a continuation of U.S. application Ser. No. 18/824,292, filed Sep. 4, 2024, which is a continuation of U.S. application Ser. No. 17/292,075, filed May 7, 2021, which is a U.S. National Phase filing of International Application Serial No. PCT/IB2019/059632, filed Nov. 8, 2019, and claims the benefit of and priority to U.S. Provisional Application No. 62/758,323, filed Nov. 9, 2018, and U.S. Provisional Application No. 62/874,459, filed Jul. 15, 2019, the entire contents of each of which are incorporated herein by reference in their entireties.

### SEQUENCE LISTING

The present application is being filed along with a Sequence Listing in electronic format. The Sequence Listing is provided as a file entitled PAT058330-US-CNT_SL.xml created Sep. 4, 2024, which is 3 kilobytes in size. The information in the electronic format of the sequence listing is incorporated herein by reference in its entirety.

### FIELD OF DISCLOSURE

The present disclosure is directed to methods of reducing the risk of a cardiovascular event with conjugated antisense compounds targeting apo(a). Specifically, a method of reducing the risk of a cardiovascular event in a patient who has established cardiovascular disease with conjugated antisense compound ISIS 681257 or a salt thereof.

### BACKGROUND OF THE DISCLOSURE

The principle behind antisense technology is that an antisense compound hybridizes to a target nucleic acid and modulates the amount, activity, and/or function of the target nucleic acid. For example in certain instances, antisense compounds result in altered transcription or translation of a target. Such modulation of expression can be achieved by, for example, target mRNA degradation or occupancy-based inhibition. An example of modulation of RNA target function by degradation is RNase H-based degradation of the target RNA upon hybridization with a DNA-like antisense compound. Another example of modulation of gene expression by target degradation is RNA interference (RNAi). RNAi refers to antisense-mediated gene silencing through a mechanism that utilizes the RNA-induced silencing complex (RISC). An additional example of modulation of RNA target function is by an occupancy-based mechanism such as is employed naturally by microRNA. MicroRNAs are small non-coding RNAs that regulate the expression of protein-coding RNAs. The binding of an antisense compound to a microRNA prevents that microRNA from binding to its messenger RNA targets, and thus interferes with the function of the microRNA. MicroRNA mimics can enhance native microRNA function. Certain antisense compounds alter splicing of pre-mRNA. Regardless of the specific mechanism, sequence-specificity makes anti sense compounds attractive as tools for target validation and gene functionalization, as well as therapeutics to selectively modulate the expression of genes involved in the pathogenesis of diseases.

Antisense technology is an effective means for modulating the expression of one or more specific gene products and can therefore prove to be uniquely useful in a number of therapeutic, diagnostic, and research applications. Chemically modified nucleosides may be incorporated into antisense compounds to enhance one or more properties, such as nuclease resistance, pharmacokinetics or affinity for a target nucleic acid. In 1998, the antisense compound, Vitravene® (fomivirsen; developed by Isis Pharmaceuticals Inc., Carlsbad, CA) was the first antisense drug to achieve marketing clearance from the U.S. Food and Drug Administration (FDA), and is currently a treatment of cytomegalovirus (CMV)-induced retinitis in AIDS patients.

New chemical modifications have improved the potency and efficacy of antisense compounds, uncovering the potential for oral delivery as well as enhancing subcutaneous administration, decreasing potential for side effects, and leading to improvements in patient convenience. Chemical modifications increasing potency of antisense compounds allow administration of lower doses, which reduces the potential for toxicity, as well as decreasing overall cost of therapy. Modifications increasing the resistance to degradation result in slower clearance from the body, allowing for less frequent dosing. Different types of chemical modifications can be combined in one compound to further optimize the compound's efficacy.

Lipoproteins are globular, micelle-like particles that consist of a non-polar core of acylglycerols and cholesteryl esters surrounded by an amphiphilic coating of protein, phospholipid and cholesterol. Lipoproteins have been classified into five broad categories on the basis of their functional and physical properties: chylomicrons, very low density lipoproteins (VLDL), intermediate density lipoproteins (IDL), low density lipoproteins (LDL), and high density lipoproteins (HDL). Chylomicrons transport dietary lipids from intestine to tissues. VLDLs, IDLs and LDLs all transport triacylglycerols and cholesterol from the liver to tissues. HDLs transport endogenous cholesterol from tissues to the liver.

Lipoprotein particles undergo continuous metabolic processing and have variable properties and compositions. Lipoprotein densities increase without increasing particle diameter because the density of their outer coatings is less than that of the inner core. The protein components of lipoproteins are known as apolipoproteins. At least nine apolipoproteins are distributed in significant amounts among the various human lipoproteins.

The lipoprotein (a) [Lp(a)] particle was identified nearly 50 years ago and is comprised of a highly unique LDL particle in which one apolipoprotein B (apoB) protein is linked via a disulfide bond to a single apolipoprotein (a) [apo(a)] protein. The apo(a) protein shares a high degree of homology with plasminogen particularly within the kringle IV type 2 repetitive domain. Levels of circulating Lp(a) are inversely proportional to the number of kringle IV type 2 variable repeats present in the molecule and, as both alleles are co-expressed within individuals, can display heterozygous plasma isoform profiles (Kraft et al., Eur J Hum. Genet, 1996; 4 (2): 74-87). It is thought that this kringle repeat domain in apo(a) may be responsible for its pro-thrombotic and anti-fibrinolytic properties, potentially enhancing atherosclerotic progression.

Apo(a) is transcriptionally regulated by IL-6 and in studies in rheumatoid arthritis patients treated with an IL-6 inhibitor (tocilizumab), plasma levels were reduced by 30% after 3 month treatment (Schultz et al., PLOS One 2010; 5: e14328).

Apo(a) has been shown to preferentially bind oxidized phospholipids and potentiate vascular inflammation (Bergmark et al., J Lipid Res 2008; 49:2230-2239; Tsimikas et al., Circulation. 2009; 119 (13): 1711-1719).

Further, studies suggest that the Lp(a) particle may also stimulate endothelial permeability, induce plasminogen activator inhibitor type-I expression and activate macrophage interleukin-8 secretion (Koschinsky and Marcovina, Curr. Opin. Lipidol 2004; 15:167-174). Importantly, recent genetic association studies revealed that Lp(a) was an independent risk factor for myocardial infarction, stroke, peripheral vascular disease and abdominal aortic aneurysm (Rifai et al., Clin. Chem. 2004; 50:1364-71; Erqou et al., JAMA 2009; 302:412-23; Kamstrup et al., Circulation 2008; 117:176-84). Further, in the recent Precocious Coronary Artery Disease (PROCARDIS) study, Clarke et al. (Clarke et al., NEJM (2009) 361; 2518-2528) described robust and independent associations between coronary heart disease and plasma Lp(a) concentrations. Additionally, Solfrizzi et al., suggested that increased serum Lp(a) may be linked to an increased risk for Alzheimer's Disease (AD) (Solfrizzi et al., J Neural Neurosurg Psychiatry 2002, 72:732-736. Currently, in the clinic setting, examples of indirect apo(a) inhibitors for treating cardiovascular disease include aspirin, Niaspan, Mipomersen, Anacetrapib, Epirotirome and Lomitapide which reduce plasma Lp(a) levels by 18%, 39%, 32%, 36%, 43% and 17%, respectively. Additionally, Lp(a) apheresis has been used in the clinic to reduce apo(a) containing Lp(a) particles.

To date, therapeutic strategies to treat cardiovascular disease by directly targeting apo(a) levels have been limited. Ribozyme oligonucleotides (U.S. Pat. No. 5,877,022) and antisense oligonucleotides (WO2005/000201; WO 2003/014397; WO 2013/177468; US20040242516; U.S. Pat. Nos. 8,138,328, 8,673,632 and 7,259,150; Merki et al., J Am Coll Cardiol 2011; 57:1611-1621; each publication incorporated by reference in its entirety) have been developed but none have been approved for commercial use.

Tsimikas et al. (Lancet. 2015 Oct. 10; 386:1472-83) discloses the results of a randomized, doubleblind, placebo-controlled Phase 1 study using an antisense compound targeting human Apo(a): ISIS 494372 (also known as ISIS-APO (a) Rx).

One chemical modification used to improve the activity of RNAse H dependent (gapmer) antisense compounds, including Apo(a) targeting compounds, in vivo is conjugation to a conjugate group, such as a GalNAc cluster. Conjugation to a conjugate group has been shown to improve potency in vivo in non-human subjects, for example including the use of RNAse H dependent (gapmer) antisense compounds conjugated to GalNAc clusters as disclosed in WO 2014/179620. Prior to the present invention, no RNAse H dependent (gapmer) antisense compounds conjugated to GalNAc clusters had been tested in humans to achieve target reduction.

WO 2014/179625 discloses antisense compounds conjugated to GalNAc clusters targeting Apo(a), including ISIS 681257.

The compound code “ISIS 681257” refers to a compound having the following structure, which code ISIS 681257 includes the compound as well as salts thereof:

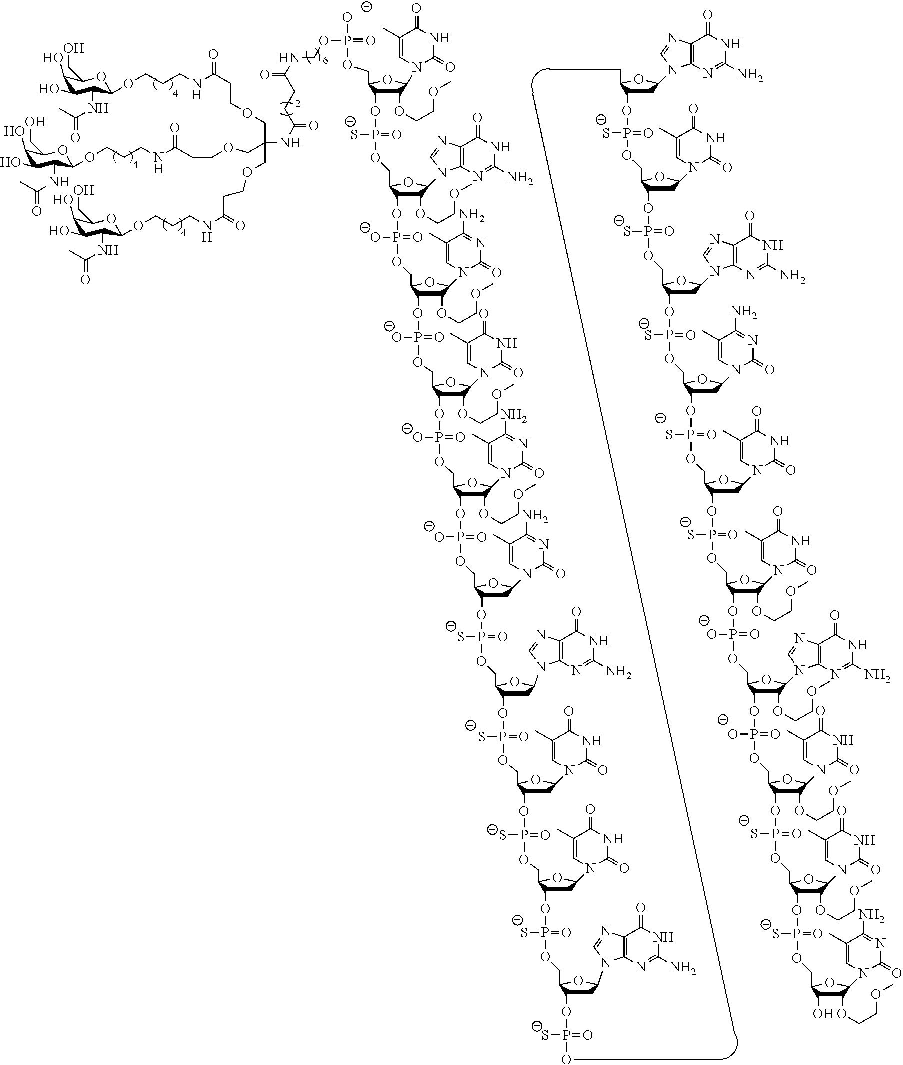

Pictured below is an example of a salt of ISIS 681257:

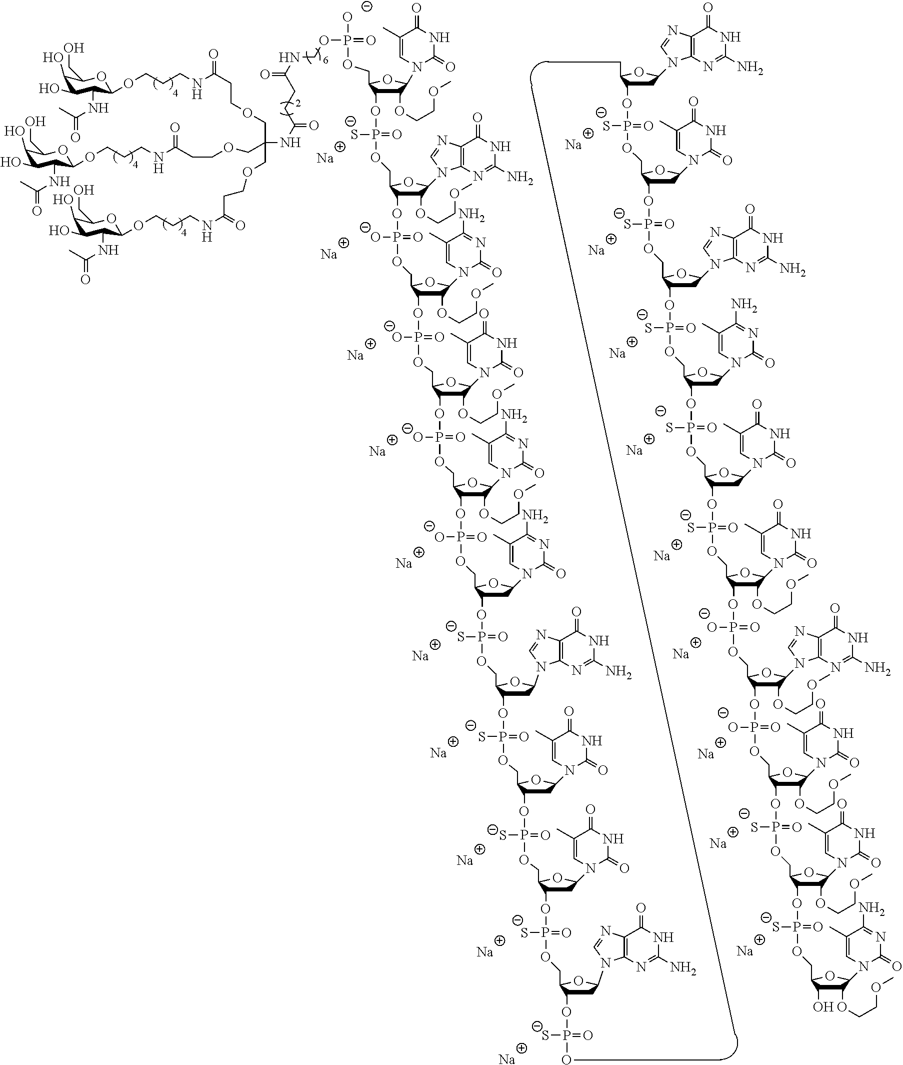

ISIS 681257 comprises a modified oligonucleotide having the nucleobase sequence TGCTCCGTTGGTGCTTGTTC (SEQ ID NO.: 1), a 5-10-5 gapmer motif, and a GalNAc conjugate.

WO 2017/079739 discloses method of treatments using ISIS 681257 at certain dosages and in certain dosing regimens. WO 2017/079739 discloses that when administered to humans, ISIS 681257 is particularly efficacious at lowering Apo(a) mRNA and plasma Lp(a) levels in terms of both its potency and its duration of action. In particular, ISIS 681257 showed a >30-fold increase in humans compared to a modified oligonucleotide having the same nucleobase sequence and the same 5-10-5 gapmer motif, but lacking a GalNAc conjugate, namely ISIS 494372. It was disclosed that ISIS 681257 provided excellent reduction of Apo(a) mR A and plasma Lp(a) and enables efficacious dosing of once a week, once a month, once every two months, or once every three months.

However, there still remains an unmet medical need for optimized treatment methods using ISIS 681257 to potently and selectively reduce Apo(a) levels in humans whilst exposing patients to only the minimum required dose on an optimized administration scheme, including in patients at enhanced risk for cardiovascular events due to chronically elevated plasma Lp(a) levels. Advantages of such optimized treatment include, e.g. reduced cost of treatment, improved patient compliance, reduced volume of administered medicinal product and/or potentially reduced risk of potential adverse events via lower dose administration regimens.

### SUMMARY OF THE DISCLOSURE

The present disclosure relates to methods of reducing the risk of a cardiovascular event in a patient who has established cardiovascular disease comprising administering the oligomeric compound ISIS 681257 to the patient at a certain dosage at a certain dosing interval.

A Phase 2B trial using ISIS 681257 (Clinical trials NCT03070782) showed that the compound considerably reduced Lp(a) levels in patients with established cardiovascular disease (CVD) and elevated levels of lipoprotein (a) with a favorable safety and tolerability profile.

The Phase 2 study was designed to evaluate the safety and tolerability of ISIS 681257 and to determine the appropriate dosing for a planned Phase 3 cardiovascular outcomes study. The randomized, double-blind, placebo-controlled, dose-ranging Phase 2 study included 286 patients with established CVD and high Lp(a) levels (baseline mean of approximately 100 mg/dl [250 nmol/L]-more than three times the upper limit of normal).

The trial had five cohorts: 20 mg (every 4 weeks), 40 mg (every 4 weeks), 60 (every 4 weeks), 20 mg (every 2 weeks), and 20 mg (every week).

The primary efficacy endpoint was the percent change in Lp(a) from baseline at the primary analysis time point (6 months) compared to placebo.

The secondary efficacy endpoints were mean percent change in LDL-C, apoB, OxPL-apoB, OxPL-apo(a), and the number of patients reaching pre-specific thresholds of <125 nmol/L (<50 mg/dL) or <75 nmol/L (<30 mg/dL).

All patients were treated for at least six months, with some patients treated up to one year.

The study met all primary and secondary efficacy endpoints analyzed at 6 months.

Results from the study show statistically significant and dose dependent reductions from baseline in Lp(a) levels:

| | | 20 mg | 40 mg | 20 mg | 60 mg | |
|---|---|---|---|---|---|---|
| | Pooled | every 4 | every 4 | every 2 | every 4 | 20 mg |
| | placebo | weeks | weeks | weeks | weeks | weekly |
| Lp(a) | (n = 47) | (n = 48) | (n = 48) | (n = 48) | (n = 47) | (n = 48) |
| LSMean % | −6 | −35 | −56 | −58 | −72 | −80 |
| change in | | (p = 0.0032) | (p < 0.0001) | (p < 0.0001) | (p < 0.0001) | (p < 0.0001) |
| Lp(a) | | | | | | |

*LSMean: Least squares mean

Approximately 98% of patients in the 20 mg weekly cohort and approximately 81% of patients in the 60 mg every 4 week cohort achieved clinically significant reductions in Lp(a) levels bringing them below the recommended threshold of risk for CVD events (<50 mg/dL).
Treatment with ISIS 681257 was associated with decreases in LDL-C, apoB, OxPL-apoB, OxPL-apo(a).
Most adverse events were mild. The most frequent adverse events were injection site reactions (ISRs). ISRs occurred in 26% of patients and were mostly mild and one patient discontinued due to an ISR.
There were no safety concerns related to platelet counts, liver function or renal function.
No patient in the study experienced a confirmed platelet count below 100,000/mm³. The incidence of platelet levels below normal (140,000/mm³) was comparable between the active (10.5%) and placebo (14.9%) groups.
Approximately 90% of patients completed treatment and the rate of discontinuation was comparable between the active (12.1%) and placebo (14.9%) groups.

These data showed that ISIS 681257 significantly reduces Lp(a) in patients with pre-existing cardiovascular disease due to elevated Lp(a) levels. ISIS 681257 is the first and only drug to show a clinically significant reduction of Lp(a) levels and a favorable safety and tolerability profile in patients with this genetic condition.

Based on these results, a new 75 mg to 85 mg, e.g. an 80 mg, dosage of ISIS 681257 is now chosen to provide maximal efficacy with an acceptable safety profile. A once monthly regimen for this dosage will decrease the overall burden to the patient associated with more frequent dosing and will provide better local tolerability.

The maximal dose evaluated in the phase 2b study was 20 mg once weekly (QW) (total monthly exposure 80 mg). The 80 mg once monthly (QM) dose is expected to provide similar efficacy to the 20 mg QW regimen, as the overall monthly exposure is comparable. This is supported by the similarity in efficacy of ISIS 681257 observed at doses of 40 mg Q4W and 20 mg Q2W. The overall exposure and reductions in Lp(a) between these regimens were similar; therefore, total monthly exposure to ISIS 681257 is believed to account for the efficacy of the drug. With regards to safety, there were no ISIS 681257-related changes in any safety signals in humans at doses ranging from 20 mg/month to 20 mg/week (equivalent to 80 mg/month; total dose ranging from 260 to 1040 mg) relative to placebo.

Accordingly, in a first aspect, the present disclosure relates to a method of reducing the risk of a cardiovascular event in a patient who has established cardiovascular disease comprising administering to said patient a unit dose comprising from about 75 mg to about 85 mg of the compound ISIS 681257 (the compound itself or a salt thereof), by subcutaneous injection to the patient once a month or every four weeks, wherein said patient has a plasma Lp(a) concentration greater than or equal to 70 mg/dL prior to the time of the first administration of the compound.

This new treatment regimen with a reduced dose of only 75 mg to about 85 mg of the compound ISIS 681257 once monthly or once every four weeks which has been shown to provide maximal efficacy with an acceptable safety profile. The once monthly regimen also decreases the overall burden to the patient associated with more frequent dosing and provides better local tolerability.

This dosing schedule is surprising, because the maximal dose evaluated in the Phase 2b study was 20 mg once weekly (QW) (equaling a total monthly exposure of 80 mg). The now described 80 mg once monthly (QM) dose provides similar efficacy to the 20 mg QW regimen. It was not necessarily to be expected that the overall monthly exposure is comparable, but it is supported by the similarity in efficacy of the compound observed at doses of 40 mg Q4W and 20 mg Q2W. The Phase 2b showed that the overall exposure and reductions in Lp(a) between these regimens were similar; therefore, total monthly exposure to the compound is believed to account for the efficacy of the drug. This total monthly exposure then allows to reduce the injection frequency to once monthly instead of once weekly.

This new treatment regimen provides one or more very significant improvements in treating humans, e.g. reduced cost of treatment, improved patient compliance, reduced volume of administered medicinal product and/or potentially reduced risk of potential adverse events via lower dose administration regimens.

The present disclosure provides the following non-limiting embodiments:

In some embodiments, the cardiovascular event is selected from a major adverse cardiovascular event (MACE), all cause death (death from any cause), coronary heart disease (CHD) death, acute myocardial infarction (AMI) death, heart failure (HF) death, death caused by the immediate complications of a cardiac procedure, and urgent lower limb re-vascularization or amputation for ischemia.

In some embodiments, the major adverse cardiovascular event (MACE) is selected from cardiovascular (CV) death, non-fatal myocardial infarction, non-fatal stroke, and urgent coronary re-vascularization requiring hospitalization. In another embodiment, the major adverse cardiovascular event (MACE) is cardiovascular (CV) death. In yet another embodiment, the major adverse cardiovascular event (MACE) is non-fatal myocardial infarction. In another embodiment, the major adverse cardiovascular event (MACE) is non-fatal stroke. In yet another embodiment, the major adverse cardiovascular event (MACE) is urgent coronary re-vascularization requiring hospitalization.

In some embodiments, the cardiovascular event is selected from all cause death (death from any cause), coronary heart disease (CHD) death, acute myocardial infarction (AMI) death, heart failure (HF) death, death caused by the immediate complications of a cardiac procedure, and urgent lower limb re-vascularization or amputation for ischemia.

In one embodiment, the cardiovascular event is all cause death (death from any cause).

In another embodiment, the cardiovascular event is coronary heart disease (CHD) death. In one embodiment, the coronary heart disease (CHD) death comprises acute myocardial infarction (AMI) death, heart failure (HF) death, and death caused by the immediate complications of a cardiac procedure.

In another embodiment, the cardiovascular event is urgent lower limb re-vascularization or amputation for ischemia.

In some embodiments, the patient who has established cardiovascular disease is a patient having at least one of the following (i) a history of spontaneous myocardial infarction, (i) a history of ischemic stroke, and (iii) clinically significant symptomatic peripheral artery disease.

In one embodiment, the history of spontaneous myocardial infarction occurred ≥3 months and ≤10 years prior to the time of the first administration of the compound.

In one embodiment, the history of ischemic stroke occurred ≥3 months and ≤10 years prior to the time of the first administration of the compound.

In one embodiment, the history of ischemic stroke is an acute episode of focal cerebral, spinal, or retinal dysfunction caused by infarction of central nervous system tissue.

In one embodiment, the clinically significant symptomatic peripheral artery disease is evidenced by intermittent claudication with at least one of (i) an ankle-brachial index ≤0.90; and (ii) lower limb amputation or re-vascularization due to lower limb ischemia.

In some embodiments, the patient has a plasma Lp(a) concentration ≥90 mg/dL prior to the time of the first administration of the compound.

In some embodiments, the unit dose comprises 75 mg to 85 mg of the compound.

In one embodiment, the unit dose comprises about 80 mg of the compound. In another embodiment, the unit dose comprises not more than 80 mg of the compound. In yet another embodiment, the unit dose comprises 80 mg of the compound.

In some embodiments, the compound is formulated in a sterile liquid and wherein each unit dose of the compound does not comprise more than 1 mL of the sterile liquid.

In one embodiment, each unit dose of the compound does not comprise more than 0.8 mL of the sterile liquid. In another embodiment, each unit dose of the compound does not comprise more than 0.5 mL of the sterile liquid. In yet another embodiment, each unit dose of the compound does not comprise more than 0.4 mL of the sterile liquid. In another embodiment, each unit dose of the compound does not comprise not more than 0.25 mL of the sterile liquid. In yet another embodiment, each unit dose of the compound does not comprise not more than 0.2 mL of the sterile liquid.

In one embodiment, the sterile liquid is water. In another embodiment, the sterile liquid is water with a sodium phosphate buffer. In yet another embodiment, the sterile liquid is water with a sodium phosphate buffer and sodium chloride.

In some embodiments, the mean/median plasma Lp(a) concentration in the patient is reduced by at least 50%, when the plasma Lp(a) concentration in the patient is measured at the start and the end of the period when the patient is dosed with the compound (dosing period). In another embodiment, the mean/median plasma Lp(a) concentration in the patient is reduced by at least 60%, when the plasma Lp(a) concentration in the patient is measured at the start and end of the dosing period. In yet another embodiment, the mean/median plasma Lp(a) concentration in the patient is reduced by at least 70%, when the plasma Lp(a) concentration in the patient is measured at the start and end of the dosing period. In another embodiment, the mean/median plasma Lp(a) concentration in the patient is reduced by at least 75%, when the plasma Lp(a) concentration in the patient is measured at the start and end of the dosing period.

In some embodiments, the overall risk of the patient to experience a major adverse cardiovascular event (MACE) is reduced by a statistically significant amount at the end of the dosing period in comparison to patients who were not administered the compound.

In other embodiments, the overall risk of the patient to experience one of the following events is reduced by a statistically significant amount at the end of the dosing period in comparison to patients who were not administered the compound: (i) the composite of cardiovascular (CV) death, non-fatal MI and non-fatal stroke; (ii) the composite of coronary heart disease (CHD) death, non-fatal MI and urgent coronary re-vascularization requiring hospitalization; (iii) the composite of coronary heart disease (CHD) death, non-fatal MI, urgent coronary re-vascularization requiring hospitalization and urgent lower limb re-vascularization or amputation for ischemia; and (iv) the rate of all cause death.

In other embodiments, the overall risk of the patient to experience one of the following events is reduced by a statistically significant amount at the end of the dosing period in comparison to patients who were not administered the compound, and wherein the patient has a plasma Lp(a) concentration ≥90 mg/dL prior to the time of the first administration of the compound: (i) the composite of all-cause mortality, non-fatal MI and non-fatal stroke; (ii) the composite of total vascular events: CV death, non-fatal MI, non-fatal stroke, urgent coronary re-vascularization requiring hospitalization and urgent lower limb re-vascularization or amputation for ischemia; (iii) the composite of all-cause mortality, non-fatal MI, non-fatal stroke and urgent coronary re-vascularization requiring hospitalization; (iv) the composite of fatal and non-fatal stroke, (v) the rate of major adverse limb events (MALE) in patients with history of peripheral artery disease (PAD), (vi) the rate of hospitalization for unstable angina, and (vii) the rate of hospitalizations for heart failure.

In one embodiment, the relative risk reduction rate (i.e., the statistically significant relative amount by which the overall risk is reduced) is at least 15% for any one of the events for a patient having a plasma Lp(a) concentration greater than or equal to 70 mg/dL prior to the time of the first administration of the compound. In another embodiment, the relative risk reduction rate (i.e., the statistically significant relative amount by which the overall risk is reduced) for any one of the events is (i) at least 15%, preferably at least 20%, more preferably at least 25%, for a patient having a plasma Lp(a) concentration greater than or equal to 70 mg/dL prior to the time of the first administration of the compound; (ii) at least 20%, preferably at least 25%, more preferably at least 30%, for a patient having a plasma Lp(a) concentration greater than or equal to 90 mg/dL prior to the time of the first administration of the compound.

In one embodiment of the above embodiments, the absolute risk reduction rate (i.e., the statistically significant absolute amount by which the overall risk is reduced) for any one of the events is (i) at least 2.0%, preferably at least 2.5%, for a patient having a plasma Lp(a) concentration greater than or equal to 70 mg/dL prior to the time of the first administration of the compound; (ii) at least 3.0%, preferably at least 3.5%, for a patient having a plasma Lp(a) concentration greater than or equal to 90 mg/dL prior to the time of the first administration of the compound.

In other embodiments, the patient shows an improvement in any one of the following events or characteristics by a statistically significant amount at the end of the dosing period in comparison to patients who were not administered the compound, and wherein the patient has a plasma Lp(a) concentration ≥90 mg/dL prior to the time of the first administration of the compound: (i) the change in Lp(a) (in mg/dL and nmol/L) from baseline at specified time points selected from 1, 2, 3, 4, 5, 6, 9, 12, 13, 15, 18, 21, 24 and 27 months after treatment initiation, (ii) the change in expanded lipid profile parameters (total cholesterol, LDL-C, apoB, HDL-C, non-HDL-C, triglycerides) and hsCRP, (iii) the incidence of new onset type 2 diabetes mellitus, (iv) the quality of life as evaluated by the SF-12 questionnaire, and (v) the time to the first occurrence of the aortic valve replacement (open or trans-catheter) or hospitalization for aortic valve stenosis.

In one embodiment thereof, the relative improvement rate (i.e., the statistically significant relative amount by which the event or characteristic is improved) is at least 15% for any one of the events or characteristics.

In some embodiments, the dosing period is at least six months. In another embodiment, the dosing period is at least one year. In yet another embodiment, the dosing period is at least two years. In another embodiment, the dosing period is at least three years.

In some embodiments, the patient receives a background therapy to achieve a guideline defined target low-density lipoprotein cholesterol (LDL-cholesterol) level.

In some embodiments, the background therapy comprises at least one of the following (i) a statin, (ii) ezetimibe, and (iii) a PCSK9 inhibitor.

In one embodiment, the background therapy comprises a statin and the patient receives an optimal dose of the statin before first administration of the compound.

In some embodiments, the patient has a sitting systolic blood pressure (SBP) less than 180 mmHg and/or diastolic BP (DBP) less than 110 mmHg.

In some embodiments, the patient has not been treated with niacin within a three month time period prior to the time of the first administration of the compound.

In some embodiments, the patient has not been diagnosed with heart failure New York Heart Association (NYHA) Class IV at the time of the first administration of the compound.

In some embodiments, the patient does not have a history of hemorrhagic stroke or other major bleeding prior to the time of the first administration of the compound.

In some embodiments, the patient has not had a myocardial infarction, stroke, coronary or lower limb re-vascularization, major cardiac or non-cardiac surgery, or lipoprotein apheresis within 3 months of the time of the first administration of the compound.

In some embodiments, the patient has no known active infection or major hematologic, renal, metabolic, gastrointestinal, or endocrine dysfunction.

In some embodiments, the patient has an estimated glomerular filtration rate (eGFR) greater than 30 mL/min/1.73 m² prior to the time of the first administration of the compound.

In some embodiments, the patient does not have an estimated glomerular filtration rate (eGFR) smaller than 30 mL/min/1.73 m² prior to the time of the first administration of the compound.

In some embodiments, the patient does not have active liver disease or hepatic dysfunction defined as aspartate aminotransferase (AST) or alanine aminotransferase (ALT) serum level more than 2 times the upper limit of normal (ULN) prior to the time of the first administration of the compound.

In some embodiments, the patient does not have a total bilirubin of more than 1.5 times the upper limit of normal (ULN) prior to the time of the first administration of the compound.c

The present disclosure provides methods comprising administering ISIS 681257 to a patient in need thereof. In certain embodiments, a patient in need thereof is a human with elevated Apo(a) levels, for example, a human having apo(a) levels ≥30 mg/dL, ≥35 mg/dL, ≥40 mg/dL, ≥50 mg/dL, ≥60 mg/dL, ≥70 mg/dL, ≥80 mg/dL, ≥90 mg/dL, ≥100 mg/dL, ≥110 mg/dL, ≥120 mg/dL, ≥130 mg/dL, ≥140 mg/dL, ≥150 mg/dL, ≥160 mg/dL, ≥170 mg/dl, ≥175 mg/dl, ≥ 180 mg/dL, ≥190 mg/dL, or ≥200 mg/dL. Lp(a) may also be expressed in nanomoles per liter. For example, a human subject having ≥75 nanomoles/liter (nmol/L) or ≥30 mg/dL, would be considered at risk of one or more cardiovascular events.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar to or equivalent to those described herein can be used in the practice and testing of the disclosure, suitable methods and materials are described below. All publications, patent applications, patents, and other references mentioned herein are incorporated by reference. The references cited herein are not admitted to be prior art to the claimed disclosure. In the case of conflict, the present specification, including definitions, will control. In addition, the materials, methods, and examples are illustrative only and not intended to be limiting.

Other features and advantages of the disclosure will be apparent from the following detailed description and claims.

### BRIEF DESCRIPTION OF THE DRAWINGS

FIG. 1 provides a summary of the study design

FIG. 2 depicts statin treatment regimen for patients.

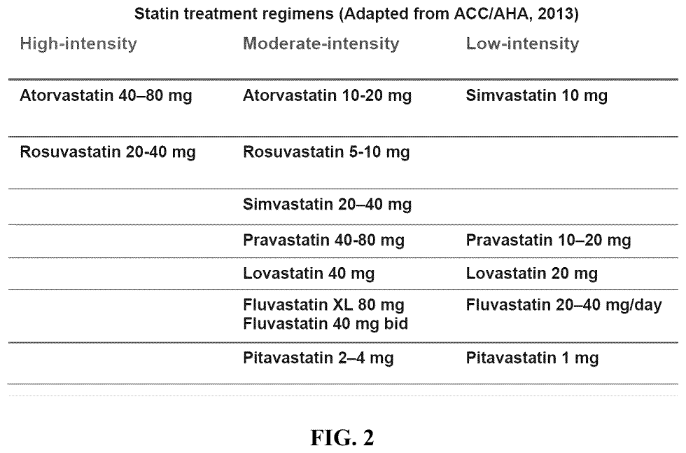

FIG. 3 depicts testing procedure for primary and secondary endpoints.

### DETAILED DESCRIPTION OF THE DISCLOSURE

The present disclosure relates to methods of reducing the risk of a cardiovascular event in a patient who has established cardiovascular disease comprising, administering of the oligomeric compound ISIS 681257 to the patient with certain specified limitations.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### Definition of Terms and Conventions Used

Unless specific definitions are provided, the nomenclature used in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis. Certain such techniques and procedures may be found for example in “Carbohydrate Modifications in Antisense Research” Edited by Sangvi and Cook, American Chemical Society, Washington D.C., 1994; “Remington's Pharmaceutical Sciences,” Mack Publishing Co., Easton, Pa., 21st edition, 2005; and “Antisense Drug Technology, Principles, Strategies, and Applications” Edited by Stanley T. Crooke, CRC Press, Boca Raton, Florida; and Sambrook et al., “Molecular Cloning, A laboratory Manual,” 2nct Edition, Cold Spring Harbor Laboratory Press, 1989, which are hereby incorporated by reference for any purpose. Where permitted, all patents, applications, published applications and other publications and other data referred to throughout in the disclosure are incorporated by reference herein in their entirety.

Unless otherwise indicated, the following terms have the following meanings:

As used herein, “dosing period” means the period of time between when a human subject receives the first dose and when the human subject receives a final dose. It is envisaged that dosing of the patient may continue after the end of the dosing period, such that a first dosing period is followed by one or more further dosing periods during which the same of a different dosing regimen is used. For example, a human subject may receive 6 doses in a first dosing period where the first and last dose are given 4 weeks apart. Subsequently, the human subject may then start a second dosing period where the human subject receives doses at regular intervals (e.g. one unit dose per week, one unit dose per month, or one unit dose per quarter).

As used herein, the term “unit dose” refers to the specific amount of the oligomeric compound administered to the human at a particular time point (e.g. the specific amount of the oligomeric compound administered to the human in a single subcutaneous injection). Each unit dose forms part of a multi-dose regimen, as described herein.

As used herein, the term “unit dosage form” denotes the physical form in which each unit dose is presented for administration.

As used here, the term “sterile liquid” means and liquid suitable for administration to a human subject. In certain embodiments, sterile liquids comprise liquids that are substantially free from viable microorganisms or bacteria. In certain embodiments, sterile liquids comprise USP grade water or USP grade saline.

As used herein, “nucleoside” means a compound comprising a nucleobase moiety and a sugar moiety. Nucleosides include, but are not limited to, naturally occurring nucleosides (as found in DNA and RNA) and modified nucleosides. Nucleosides may be linked to a phosphate moiety.

As used herein, “chemical modification” means a chemical difference in a compound when compared to a naturally occurring counterpart. Chemical modifications of oligonucleotides include nucleoside modifications (including sugar moiety modifications and nucleobase modifications) and internucleoside linkage modifications. In reference to an oligonucleotide, chemical modification does not include differences only in nucleobase sequence.

As used herein, “furanosyl” means a structure comprising a 5-membered ring comprising four carbon atoms and one oxygen atom.

As used herein, “naturally occurring sugar moiety” means a ribofuranosyl as found in naturally occurring RNA or a deoxyribofuranosyl as found in naturally occurring DNA.

As used herein, “sugar moiety” means a naturally occurring sugar moiety or a modified sugar moiety of a nucleoside.

As used herein, “modified sugar moiety” means a substituted sugar moiety or a sugar surrogate.

As used herein, “substituted sugar moiety” means a furanosyl that is not a naturally occurring sugar moiety. Substituted sugar moieties include, but are not limited to furanosyls comprising substituents at the 2′-position, the 3′-position, the 5′-position and/or the 4′-position. Certain substituted sugar moieties are bicyclic sugar moieties.

As used herein, “2′-substituted sugar moiety” means a furanosyl comprising a substituent at the 2′-position other than Hor OH. Unless otherwise indicated, a 2′-substituted sugar moiety is not a bicyclic sugar moiety (i.e., the 2′-substituent of a 2′-substituted sugar moiety does not form a bridge to another atom of the furanosyl ring.

As used herein, “MOE” means —OCH₂CH₂OCH₃.

As used herein, “nucleic acid” refers to molecules composed of monomeric nucleotides. A nucleic acid includes ribonucleic acids (RNA), deoxyribonucleic acids (DNA), single-stranded nucleic acids (ssDNA), double-stranded nucleic acids (dsDNA), small interfering ribonucleic acids (siRNA), and microRNAs (miRNA). A nucleic acid may also comprise any combination of these elements in a single molecule.

As used herein, “nucleotide” means a nucleoside further comprising a phosphate linking group. As used herein, “linked nucleosides” may or may not be linked by phosphate linkages and thus includes, but is not limited to “linked nucleotides.” As used herein, “linked nucleosides” are nucleosides that are connected in a continuous sequence (i.e. no additional nucleosides are present between those that are linked).

As used herein, “nucleobase” means a group of atoms that can be linked to a sugar moiety to create a nucleoside that is capable of incorporation into an oligonucleotide, and wherein the group of atoms is capable of bonding with a complementary naturally occurring nucleobase of another oligonucleotide or nucleic acid. Nucleobases may be naturally occurring or may be modified. As used herein, “nucleobase sequence” means the order of contiguous nucleobases independent of any sugar, linkage, or nucleobase modification.

As used herein the terms, “unmodified nucleobase” or “naturally occurring nucleobase” means the naturally occurring heterocyclic nucleobases of RNA or DNA: the purine bases adenine (A) and guanine (G), and the pyrimidine bases thymine (T), cytosine (C) (including 5-methyl C), and uracil (U).

As used herein, “modified nucleobase” means any nucleobase that is not a naturally occurring nucleobase.

As used herein, “modified nucleoside” means a nucleoside comprising at least one chemical modification compared to naturally occurring RNA or DNA nucleosides. Modified nucleosides comprise a modified sugar moiety and/or a modified nucleobase.

As used herein, “2′-substituted nucleoside” means a nucleoside comprising a substituent at the 2′-position other than H or OH. Unless otherwise indicated, a 2′-substituted nucleoside is not a bicyclic nucleoside.

As used herein, “deoxynucleoside” means a nucleoside comprising 2′-H furanosyl sugar moiety, as found in naturally occurring deoxyribonucleosides (DNA). In certain embodiments, a 2′-deoxynucleoside may comprise a modified nucleobase or may comprise an RNA nucleobase (e.g., uracil).

As used herein, “oligonucleotide” means a compound comprising a plurality of linked nucleosides. In certain embodiments, an oligonucleotide comprises one or more unmodified ribonucleosides (RNA) and/or unmodified deoxyribonucleosides (DNA) and/or one or more modified nucleosides.

As used herein “oligonucleoside” means an oligonucleotide in which none of the internucleoside linkages contains a phosphorus atom. As used herein, oligonucleotides include oligonucleosides.

As used herein, “modified oligonucleotide” means an oligonucleotide comprising at least one modified nucleoside and/or at least one modified internucleoside linkage.

As used herein, “linkage” or “linking group” means a group of atoms that link together two or more other groups of atoms.

As used herein “internucleoside linkage” means a covalent linkage between adjacent nucleosides in an oligonucleotide.

As used herein “naturally occurring internucleoside linkage” means a 3′ to 5′ phosphodiester linkage.

As used herein, “modified internucleoside linkage” means any internucleoside linkage other than a naturally occurring internucleoside linkage.

As used herein, “terminal internucleoside linkage” means the linkage between the last two nucleosides of an oligonucleotide or defined region thereof.

As used herein, “phosphorus linking group” means a linking group comprising a phosphorus atom. Phosphorus linking groups include without limitation groups having the formula:

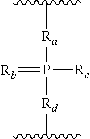

wherein:
Rₐ and R_{d} are each, independently, O, S, CH₂, NH, or NJ₁ wherein J₁ is C₁-C₆ alkyl or substituted C₁-C₆ alkyl;
R_{b} is O or S; and
R_{c} is OH, SH, C₁-C₆ alkyl, substituted C₁-C₆ alkyl, C₁-C₆ alkoxy, substituted C₁-C₆ alkoxy, amino, or substituted amino.

Phosphorus linking groups include without limitation, phosphodiester, phosphorothioate, phosphorodithioate, phosphonate, phosphoramidate, phosphorothioamidate, thionoalkylphosphonate, phosphotriesters, thionoalkylphosphotriester and boranophosphate.

As used herein, “internucleoside phosphorus linking group” means a phosphorus linking group that directly links two nucleosides.

As used herein, “non-internucleoside phosphorus linking group” means a phosphorus linking group that does not directly link two nucleosides. In certain embodiments, a non-internucleoside phosphorus linking group links a nucleoside to a group other than a nucleoside. In certain embodiments, a non-internucleoside phosphorus linking group links two groups, neither of which is a nucleoside.

As used herein, “neutral linking group” means a linking group that is not charged. Neutral linking groups include without limitation phosphotriesters, methylphosphonates, MMI (—CH₂—N(CH₃)—O—), amide-3 (—CH₂—C(═O)—N(H)—), amide-4 (—CH₂—N(H)—C(═O)—), formacetal (—O—CH₂—O—), and thioformacetal (—S—CH₂—O—). Further neutral linking groups include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook Eds. ACS Symposium Series 580; Chapters 3 and 4, (pp. 40-65)). Further neutral linking groups include nonionic linkages comprising mixed N, O, S, and CH₂ component parts.

As used herein, “internucleoside neutral linking group” means a neutral linking group that directly links two nucleosides.

As used herein, “oligomeric compound” means a polymeric structure comprising two or more substructures. In certain embodiments, an oligomeric compound comprises an oligonucleotide. In certain embodiments, an oligomeric compound comprises one or more conjugate groups and/or terminal groups. In certain embodiments, an oligomeric compound consists of an oligonucleotide. Oligomeric compounds also include naturally occurring nucleic acids. In certain embodiments, an oligomeric compound comprises a backbone of one or more linked monomeric subunits where each linked monomeric subunit is directly or indirectly attached to a heterocyclic base moiety. In certain embodiments, oligomeric compounds may also include monomeric subunits that are not linked to a heterocyclic base moiety, thereby providing abasic sites. In certain embodiments, the linkages joining the monomeric subunits, the sugar moieties or surrogates and the heterocyclic base moieties can be independently modified. In certain embodiments, the linkage-sugar unit, which may or may not include a heterocyclic base, may be substituted with a mimetic such as the monomers in peptide nucleic acids.

As used herein, “terminal group” means one or more atom attached to either, or both, the 3′ end or the 5′ end of an oligonucleotide. In certain embodiments, a terminal group is a conjugate group. In certain embodiments, a terminal group comprises one or more terminal group nucleosides.

As used herein, “conjugate” or “conjugate group” means an atom or group of atoms bound to an oligonucleotide or oligomeric compound. In general, conjugate groups modify one or more properties of the compound to which they are attached, including, but not limited to pharmacodynamic, pharmacokinetic, binding, absorption, cellular distribution, cellular uptake, charge and/or clearance properties.

As used herein, “conjugate linker” or “linker” in the context of a conjugate group means a portion of a conjugate group comprising any atom or group of atoms and which covalently link (1) an oligonucleotide to another portion of the conjugate group or (2) two or more portions of the conjugate group.

Conjugate groups are shown herein as radicals, providing a bond for forming covalent attachment to an oligomeric compound such as an antisense oligonucleotide. In certain embodiments, the point of attachment on the oligomeric compound is the 3′-oxygen atom of the 3′-hydroxyl group of the 3′ terminal nucleoside of the oligomeric compound. In certain embodiments, the point of attachment on the oligomeric compound is the 5′-oxygen atom of the 5′-hydroxyl group of the 5′ terminal nucleoside of the oligomeric compound. In certain embodiments, the bond for forming attachment to the oligomeric compound is a cleavable bond. In certain such embodiments, such cleavable bond constitutes all or part of a cleavable moiety.

In certain embodiments, conjugate groups comprise a cleavable moiety (e.g., a cleavable bond or cleavable nucleoside) and a carbohydrate cluster portion, such as a GalNAc cluster portion. Such carbohydrate cluster portion comprises: a targeting moiety and, optionally, a conjugate linker. In certain embodiments, the carbohydrate cluster portion is identified by the number and identity of the ligand. For example, in certain embodiments, the carbohydrate cluster portion comprises 3 GalNAc groups and is designated “GalNAc3”. Specific carbohydrate cluster portions (having specific tether, branching and conjugate linker groups) are described herein and designated by Roman numeral followed by subscript “a”. Accordingly “GalNac3-1ₐ” refers to a specific carbohydrate cluster portion of a conjugate group having 3 GalNac groups and specifically identified tether, branching and linking groups. Such carbohydrate cluster fragment is attached to an oligomeric compound via a cleavable moiety, such as a cleavable bond or cleavable nucleoside.

As used herein, “cleavable moiety” means a bond or group that is capable of being split under physiological conditions. In certain embodiments, a cleavable moiety is cleaved inside a cell or sub-cellular compartments, such as a lysosome. In certain embodiments, a cleavable moiety is cleaved by endogenous enzymes, such as nucleases. In certain embodiments, a cleavable moiety comprises a group of atoms having one, two, three, four, or more than four cleavable bonds.

As used herein, “cleavable bond” means any chemical bond capable of being split. In certain embodiments, a cleavable bond is selected from among: an amide, a polyamide, an ester, an ether, one or both esters of a phosphodiester, a phosphate ester, a carbamate, a di-sulfide, or a peptide.

As used herein, “carbohydrate cluster” means a compound having one or more carbohydrate residues attached to a scaffold or linker group. (see, e.g., Maier et al., “Synthesis of Antisense Oligonucleotides Conjugated to a Multivalent Carbohydrate Cluster for Cellular Targeting,” Bioconjugate Chemistry, 2003, (14): 18-29, which is incorporated herein by reference in its entirety, or Rensen et al., “Design and Synthesis of Novel N-Acetylgalactosamine-Terminated Glycolipids for Targeting of Lipoproteins to the Hepatic Asiaglycoprotein Receptor,” J Med. Chem. 2004, (47): 5798-5808, for examples of carbohydrate conjugate clusters).

As used herein, “single-stranded” means an oligomeric compound that is not hybridized to its complement and which lacks sufficient self-complementarity to form a stable self-duplex.

As used herein, “double-stranded” means a pair of oligomeric compounds that are hybridized to one another or a single self-complementary oligomeric compound that forms a hairpin structure. In certain embodiments, a double-stranded oligomeric compound comprises a first and a second oligomeric compound.

As used herein, “antisense compound” means a compound comprising or consisting of an oligonucleotide at least a portion of which is complementary to a target nucleic acid to which it is capable of hybridizing, resulting in at least one antisense activity.

As used herein, “antisense activity” means any detectable and/or measurable change attributable to the hybridization of an antisense compound to its target nucleic acid. In certain embodiments, antisense activity includes modulation of the amount or activity of a target nucleic acid transcript (e.g. mRNA). In certain embodiments, antisense activity includes modulation of the splicing of pre-mRNA.

As used herein, “RNase H based antisense compound” means an antisense compound wherein at least some of the antisense activity of the antisense compound is attributable to hybridization of the antisense compound to a target nucleic acid and subsequent cleavage of the target nucleic acid by RNase H.

As used herein, “detecting” or “measuring” means that a test or assay for detecting or measuring is performed. Such detection and/or measuring may result in a value of zero. Thus, if a test for detection or measuring results in a finding of no activity (activity of zero), the step of detecting or measuring the activity has nevertheless been performed.

As used herein, “detectable and/or measureable activity” means a statistically significant activity that is not zero.

As used herein, “essentially unchanged” means little or no change in a particular parameter, particularly relative to another parameter which changes much more. In certain embodiments, a parameter is essentially unchanged when it changes less than 5%. In certain embodiments, a parameter is essentially unchanged if it changes less than two-fold while another parameter changes at least ten-fold. For example, in certain embodiments, an antisense activity is a change in the amount of a target nucleic acid. In certain such embodiments, the amount of a non-target nucleic acid is essentially unchanged if it changes much less than the target nucleic acid does, but the change need not be zero.

As used herein, “expression” means the process by which a gene ultimately results in a protein. Expression includes, but is not limited to, transcription, post-transcriptional modification (e.g., splicing, polyadenlyation, addition of 5′-cap), and translation.

As used herein, “target nucleic acid” means a nucleic acid molecule to which an antisense compound is intended to hybridize to result in a desired antisense activity. Antisense oligonucleotides have sufficient complementarity to their target nucleic acids to allow hybridization under physiological conditions.

As used herein, “nucleobase complementarity” or “complementarity” when in reference to nucleobases means a nucleobase that is capable of base pairing with another nucleobase. For example, in DNA, adenine (A) is complementary to thymine (T). For example, in RNA, adenine (A) is complementary to uracil (U). In certain embodiments, complementary nucleobase means a nucleobase of an antisense compound that is capable of base pairing with a nucleobase of its target nucleic acid. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be complementary at that nucleobase pair. Nucleobases comprising certain modifications may maintain the ability to pair with a counterpart nucleobase and thus, are still capable of nucleobase complementarity.

As used herein, “non-complementary” in reference to nucleobases means a pair of nucleobases that do not form hydrogen bonds with one another.

As used herein, “complementary” in reference to oligomeric compounds (e.g., linked nucleosides, oligonucleotides, or nucleic acids) means the capacity of such oligomeric compounds or regions thereof to hybridize to another oligomeric compound or region thereof through nucleobase complementarity. Complementary oligomeric compounds need not have nucleobase complementarity at each nucleoside. Rather, some mismatches are tolerated. In certain embodiments, complementary oligomeric compounds or regions are complementary at 70% of the nucleobases (70% complementary). In certain embodiments, complementary oligomeric compounds or reg10 ns are 80% complementary. In certain embodiments, complementary oligomeric compounds or reg10 ns are 90% complementary. In certain embodiments, complementary oligomeric compounds or reg10 ns are 95% complementary. In certain embodiments, complementary oligomeric compounds or regions are 100% complementary.

As used herein, “mismatch” means a nucleobase of a first oligomeric compound that is not capable of pairing with a nucleobase at a corresponding position of a second oligomeric compound, when the first and second oligomeric compound are aligned. Either or both of the first and second oligomeric compounds may be oligonucleotides.

As used herein, “hybridization” means the pairing of complementary oligomeric compounds (e.g., an antisense compound and its target nucleic acid). While not limited to a particular mechanism, the most common mechanism of pairing involves hydrogen bonding, which may be Watson-Crick, Hoogsteen or reversed Hoogsteen hydrogen bonding, between complementary nucleobases.

As used herein, “specifically hybridizes” means the ability of an oligomeric compound to hybridize to one nucleic acid site with greater affinity than it hybridizes to another nucleic acid site.

As used herein, “fully complementary” in reference to an oligonucleotide or portion thereof means that each nucleobase of the oligonucleotide or portion thereof is capable of pairing with a nucleobase of a complementary nucleic acid or contiguous portion thereof. Thus, a fully complementary region comprises no mismatches or unhybridized nucleobases in either strand.

As used herein, “percent complementarity” means the percentage of nucleobases of an oligomeric compound that are complementary to an equal-length portion of a target nucleic acid. Percent complementarity is calculated by dividing the number of nucleobases of the oligomeric compound that are complementary to nucleobases at corresponding positions in the target nucleic acid by the total length of the oligomeric compound.

As used herein, “percent identity” means the number of nucleobases in a first nucleic acid that are the same type (independent of chemical modification) as nucleobases at corresponding positions in a second nucleic acid, divided by the total number of nucleobases in the first nucleic acid.

As used herein, “modulation” means a change of amount or quality of a molecule, function, or activity when compared to the amount or quality of a molecule, function, or activity prior to modulation. For example, modulation includes the change, either an increase (stimulation or induction) or a decrease (inhibition or reduction) in gene expression. As a further example, modulation of expression can include a change in splice site selection of pre-mRNA processing, resulting in a change in the absolute or relative amount of a particular splice-variant compared to the amount in the absence of modulation.

As used herein, “chemical motif” means a pattern of chemical modifications in an oligonucleotide or a region thereof. Motifs may be defined by modifications at certain nucleosides and/or at certain linking groups of an oligonucleotide.

As used herein, “nucleoside motif” means a pattern of nucleoside modifications in an oligonucleotide or a region thereof. The linkages of such an oligonucleotide may be modified or unmodified. Unless otherwise indicated, motifs herein describing only nucleosides are intended to be nucleoside motifs. Thus, in such instances, the linkages are not limited.

As used herein, “sugar motif” means a pattern of sugar modifications in an oligonucleotide or a region thereof.

As used herein, “linkage motif” means a pattern of linkage modifications in an oligonucleotide or region thereof. The nucleosides of such an oligonucleotide may be modified or unmodified. Unless otherwise indicated, motifs herein describing only linkages are intended to be linkage motifs. Thus, in such instances, the nucleosides are not limited.

As used herein, “nucleobase modification motif” means a pattern of modifications to nucleobases along an oligonucleotide. Unless otherwise indicated, a nucleobase modification motif is independent of the nucleobase sequence.

As used herein, “sequence motif” means a pattern of nucleobases arranged along an oligonucleotide or portion thereof. Unless otherwise indicated, a sequence motif is independent of chemical modifications and thus may have any combination of chemical modifications, including no chemical modifications.
As used herein, “type of modification” in reference to a nucleoside or a nucleoside of a “type” means the chemical modification of a nucleoside and includes modified and unmodified nucleosides. Accordingly, unless otherwise indicated, a “nucleoside having a modification of a first type” may be an unmodified nucleoside.

As used herein, “differently modified” mean chemical modifications or chemical substituents that are different from one another, including absence of modifications. Thus, for example, a MOE nucleoside and an unmodified DNA nucleoside are “differently modified,” even though the DNA nucleoside is unmodified. Likewise, DNA and RNA are “differently modified,” even though both are naturally-occurring unmodified nucleosides. Nucleosides that are the same but for comprising different nucleobases are not differently modified. For example, a nucleoside comprising a 2′-OMe modified sugar and an unmodified adenine nucleobase and a nucleoside comprising a 2′-OMe modified sugar and an unmodified thymine nucleobase are not differently modified.

As used herein, “the same type of modifications” refers to modifications that are the same as one another, including absence of modifications. Thus, for example, two unmodified DNA nucleosides have “the same type of modification,” even though the DNA nucleoside is unmodified. Such nucleosides having the same type modification may comprise different nucleobases.

As used herein, “separate regions” means portions of an oligonucleotide wherein the chemical modifications or the motif of chemical modifications of any neighboring portions include at least one difference to allow the separate regions to be distinguished from one another.

As used herein, “pharmaceutically acceptable carrier or diluent” means any substance suitable for use in administering to an animal. In certain embodiments, a pharmaceutically acceptable carrier or diluent is sterile saline. In certain embodiments, such sterile saline is pharmaceutical grade saline.

As used herein the term “metabolic disorder” means a disease or condition principally characterized by dysregulation of metabolism—the complex set of chemical reactions associated with breakdown of food to produce energy.

As used herein, the term “cardiovascular disorder” means a disease or condition principally characterized by impaired function of the heart or blood vessels.

As used herein, “prodrug” means an inactive or less active form of a compound which, when administered to a subject, is metabolized to form the active, or more active, compound (e.g., drug).

As used herein, “SF-12” refers to a widely used validated generic health-related quality of life (HRQOL) instrument, which encompasses generic health concepts considered to be relevant across age groups, disease states and treatments types. The measure comprises 12 items and has 8 domains: general health (1 item), physical functioning (2 items), role limitations due to physical health (2 items), bodily pain (1 item), vitality (1 item), social functioning (1 item), role limitations due to emotional problems (2 items), mental health (2 items). In addition, there are two composite summary scores derived based on the SF-12 individual domains:
Physical Component Summary (PCS)
Mental Component Summary (MCS).

As used herein, unless otherwise indicated or modified, the term “double-stranded” refers to two separate oligomeric compounds that are hybridized to one another. Such double stranded compounds may have one or more or non-hybridizing nucleosides at one or both ends of one or both strands (overhangs) and/or one or more internal non-hybridizing nucleosides (mismatches) provided there is sufficient complementarity to maintain hybridization under physiologically relevant conditions.

As used herein, “5′ target site” refers to the nucleotide of a target nucleic acid which is complementary to the 5′-most nucleotide of a particular antisense compound.

As used herein, “about” means within +10% of a value. For example, if it is stated, “a marker May 30 be increased by about 50%”, it is implied that the marker may be increased between 45%-55%.

As used herein, “administered concomitantly” refers to the co-administration of two agents in any manner in which the pharmacological effects of both are manifest in the patient at the same time. Concomitant administration does not require that both agents be administered in a single pharmaceutical composition, in the same dosage form, or by the same route of administration. The effects of both agents need not manifest themselves at the same time. The effects need only be overlapping for a period of time and need not be coextensive.

As used herein, “administering” or “administration” means providing a pharmaceutical agent to an individual, and includes, but is not limited to, administering by a medical professional and self-administering. Administration of a pharmaceutical agent to an individual can be continuous, chronic, short or intermittent. Administration can parenteral or non-parenteral.

As used herein, “agent” means an active substance that can provide a therapeutic benefit when administered to an animal. “First agent” means a therapeutic compound of the invention. For example, a first agent can be an antisense oligonucleotide targeting apo(a). “Second agent” means a second therapeutic compound of the invention (e.g. a second antisense oligonucleotide targeting apo(a)) and/or a non-apo(a) therapeutic compound.

As used herein, “amelioration” or “ameliorate” or “ameliorating” refers to a lessening of at least one indicator, sign, or symptom of an associated disease, disorder, or condition. The severity of indicators can be determined by subjective or objective measures, which are known to those skilled in the art.

As used herein, “apo(a)” means any nucleic acid or protein sequence encoding apo(a). For example, in certain embodiments, apo(a) includes a DNA sequence encoding apo(a), a RNA sequence transcribed from DNA encoding apo(a) (including genomic DNA comprising intrans and exons), a mRNA sequence encoding apo(a), or a peptide sequence encoding apo(a).

As used herein, “apo(a) nucleic acid” means any nucleic acid encoding apo(a). For example, in certain embodiments, an apo(a) nucleic acid includes a DNA sequence encoding apo(a), a RNA sequence transcribed from DNA encoding apo(a) (including genomic DNA comprising intrans and exons), and a mRNA sequence encoding apo(a).

As used herein, “apo(a) mRNA” means a mRNA encoding an apo(a) protein.

As used herein, “apo(a) protein” means any protein sequence encoding Apo(a).

As used herein, “apo(a) specific inhibitor” refers to any agent capable of specifically inhibiting the expression of an apo(a) nucleic acid and/or apo(a) protein. For example, apo(a) specific inhibitors include nucleic acids (including antisense compounds), peptides, antibodies, small molecules, and other agents capable of inhibiting the expression of apo(a) nucleic acid and/or apo(a) protein. In certain embodiments, by specifically modulating apo(a) nucleic acid expression and/or apo(a) protein expression, apo(a) specific inhibitors can affect other components of the lipid transport system including downstream components. Similarly, in certain embodiments, apo(a) specific inhibitors can affect other molecular processes in an animal.

As used herein “optimal dose of a statin” means a dose as adapted from the American College of Cardiology (ACC)/American Heart Association (AHA) guideline on the treatment of Blood Cholesterol to Reduce Atherosclerotic Cardiovascular Risk in Adults. (See Stone, Neil J. et al., “2013 ACC/AHA Guideline on the Treatment of Blood Cholesterol to Reduce Atherosclerotic Cardiovascular Risk in Adults, A Report of the American College of Cardiology/American Heart Association Task Force on Practice Guidelines,” Circulation, Jun. 24, 2014, S1-S45)

As used herein, “atherosclerosis” means a hardening of the arteries affecting large and medium-sized arteries and is characterized by the presence of fatty deposits. The fatty deposits are called “atheromas” or “plaques,” which consist mainly of cholesterol and other fats, calcium and scar tissue, and damage the lining of arteries.

As used herein, “coronary heart disease (CHD)” means a narrowing of the small blood vessels that supply blood and oxygen to the heart, which is often a result of atherosclerosis.

As used herein, “diabetes mellitus” or “diabetes” is a syndrome characterized by disordered metabolism and abnormally high blood sugar (hyperglycemia) resulting from insufficient levels of insulin or reduced insulin sensitivity. The characteristic symptoms are excessive urine production (polyuria) due to high blood glucose levels, excessive thirst and increased fluid intake (polydipsia) attempting to compensate for increased urination, blurred vision due to high blood glucose effects on the eye's optics, unexplained weight loss, and lethargy.

As used herein, “diabetic dyslipidemia” or “type 2 diabetes with dyslipidemia” means a condition characterized by Type 2 diabetes, reduced HDL-C, elevated triglycerides (TG), and elevated small, dense LDL particles.

As used herein, “diluent” means an ingredient in a composition that lacks pharmacological activity, but is pharmaceutically necessary or desirable. For example, the diluent in an injected composition can be a liquid, e.g. saline solution.

As used herein, “dyslipidemia” refers to a disorder of lipid and/or lipoprotein metabolism, including lipid and/or lipoprotein overproduction or deficiency. Dyslipidemias can be manifested by elevation of lipids such as chylomicron, cholesterol and triglycerides as well as lipoproteins such as low-density lipoprotein (LDL) cholesterol.

As used herein, “dose” means a specified quantity of a pharmaceutical agent provided in a single administration, or in a specified time period. In certain embodiments, a dose can be administered in one, two, or more boluses, tablets, or injections. For example, in certain embodiments where subcutaneous administration is desired, the desired dose requires a volume not easily accommodated by a single injection, therefore, two or more injections can be used to achieve the desired dose. In certain embodiments, the pharmaceutical agent is administered by infusion over an extended period of time or continuously. Doses can be stated as the amount of pharmaceutical agent per hour, day, week, or month. Doses can also be stated as mg/kg or g/kg.

As used herein, “effective amount” or “therapeutically effective amount” means the amount of active pharmaceutical agent sufficient to effectuate a desired physiological outcome in an individual in need of the agent. The effective amount can vary among individuals depending on the health and physical condition of the individual to be treated, the taxonomic group of the individuals to be treated, the formulation of the composition, assessment of the individual's medical condition, and other relevant factors.

As used herein, “fully complementary” or “100% complementary” means each nucleobase of a nucleobase sequence of a first nucleic acid has a complementary nucleobase in a second nucleobase sequence of a second nucleic acid. In certain embodiments, a first nucleic acid is an antisense compound and a second nucleic acid is a target nucleic acid.

As used herein, “glucose” is a monosaccharide used by cells as a source of energy and inflammatory intermediate. “Plasma glucose” refers to glucose present in the plasma.

As used herein, “high density lipoprotein-C” or “HDL-C” means cholesterol associated with high-density lipoprotein particles. Concentration of HDL-C in serum (or plasma) is typically quantified in mg/dL or nmol/L. “Serum HDL-C” and “plasma HDL-C” mean HDL-C in serum and plasma, respectively.

As used herein, “HMG-COA reductase inhibitor” means an agent that acts through the inhibition of the enzyme HMG-COA reductase, such as atorvastatin, rosuvastatin, fluvastatin, lovastatin, pravastatin, and simvastatin.

As used herein, “hypercholesterolemia” means a condition characterized by elevated cholesterol or circulating (plasma) cholesterol, LDL-cholesterol and VLDL-cholesterol, as per the guidelines of the Expert Panel Report of the National Cholesterol Educational Program (NCEP) of Detection, Evaluation of Treatment of high cholesterol in adults (see, Arch. Int. Med. (1988) 148, 36-39).

As used herein, “hyperlipidemia” or “hyperlipemia” is a condition characterized by elevated serum lipids or circulating (plasma) lipids. This condition manifests an abnormally high concentration of fats. The lipid fractions in the circulating blood are cholesterol, low-density lipoproteins, very low density lipoproteins, chylomicrons and triglycerides. The Fredrickson classification of hyperlipidemias is based on the pattern of TG and cholesterol-rich lipoprotein particles, as measured by electrophoresis or ultracentrifugation and is commonly used to characterize primary causes of hyperlipidemias such as hypertriglyceridemia (Fredrickson and Lee, Circulation, 1965, 31:321-327; Fredrickson et al., New Eng J Med, 1967, 276 (1): 34-42).

As used herein, “hypertriglyceridemia” means a condition characterized by elevated triglyceride levels. Its etiology includes primary (i.e. genetic causes) and secondary (other underlying causes such as diabetes, metabolic syndrome/insulin resistance, obesity, physical inactivity, cigarette smoking, excess alcohol and a diet very high in carbohydrates) factors or, most often, a combination of both (Yuan et al., CMAJ, 2007, 176:1113-1120).

As used herein, “identifying” or “selecting an animal with metabolic or cardiovascular disease” means identifying or selecting a subject prone to or having been diagnosed with a metabolic disease, a cardiovascular disease, or a metabolic syndrome; or, identifying or selecting a subject having any symptom of a metabolic disease, cardiovascular disease, or metabolic syndrome including, but not limited to, hypercholesterolemia, hyperglycemia, hyperlipidemia, hypertriglyceridemia, hypertension increased insulin resistance, decreased insulin sensitivity, above normal body weight, and/or above normal body fat content or any combination thereof. Such identification can be accomplished by any method, including but not limited to, standard clinical tests or assessments, such as measuring serum or circulating (plasma) cholesterol, measuring serum or circulating (plasma) blood-glucose, measuring serum or circulating (plasma) triglycerides, measuring blood-pressure, measuring body fat content, measuring body weight, and the like.

As used herein, “improved cardiovascular outcome” means a reduction in the occurrence of adverse cardiovascular events, or the risk thereof. Examples of adverse cardiovascular events include, without limitation, death, reinfarction, stroke, cardiogenic shock, pulmonary edema, cardiac arrest, and atrial dysrhythmia.

As used herein, “increasing HDL” or “raising HDL” means increasing the level of HDL in an animal after administration of at least one compound of the invention, compared to the HDL level in an animal not administered any compound.

As used herein, “individual”, “patient”, or “subject” means a human selected for treatment or therapy.

As used herein, “individual in need thereof” refers to a human or non-human animal selected for treatment or therapy that is in need of such treatment or therapy.

As used herein, “induce”, “inhibit”, “potentiate”, “elevate”, “increase”, “decrease”, “reduce” or the like denote quantitative differences between two states. For example, “an amount effective to inhibit the activity or expression of apo(a)” means that the level of activity or expression of apo(a) in a treated sample will differ from the level of apo(a) activity or expression in an untreated sample. Such terms are applied to, for example, levels of expression, and levels of activity.

The terms “lower”, “reduce”, “reduction”, “decrease” or “inhibit” are all used herein generally to mean a decrease by a statistically significant amount. However, for avoidance of doubt, “lower”, “reduce”, “reduction” or “decrease” or “inhibit” means a decrease by at least 10% as compared to a reference level, for example a decrease by at least about 10%, or at least about 15%, or at least about 20%, or at least about 25%, or at least about 30%, or at least about 35%, or at least about 40%, or at least about 50%, (i.e. absent level as compared to a reference sample), or any decrease between 10-50% as compared to a reference level.

As used herein, “inflammatory condition” refers to a disease, disease state, syndrome, or other condition resulting in inflammation. For example, rheumatoid arthritis and liver fibrosis are inflammatory conditions. Other examples of inflammatory conditions include sepsis, myocardial ischemia/reperfusion injury, adult respiratory distress syndrome, nephritis, graft rejection, inflammatory bowel disease, multiple sclerosis, arteriosclerosis, atherosclerosis and vasculitis.

As used herein, “inhibiting the expression or activity” refers to a reduction or blockade of the expression or activity of a RNA or protein and does not necessarily indicate a total elimination of expression or activity.

As used herein, “insulin resistance” is defined as the condition in which normal amounts of insulin are inadequate to produce a normal insulin response from fat, muscle and liver cells. Insulin resistance in fat cells results in hydrolysis of stored triglycerides, which elevates free fatty acids in the blood plasma. Insulin resistance in muscle reduces glucose uptake whereas insulin resistance in liver reduces glucose storage, with both effects serving to elevate blood glucose. High plasma levels of insulin and glucose due to insulin resistance often leads to metabolic syndrome and type 2 diabetes.

As used herein, “insulin sensitivity” is a measure of how effectively an individual processes glucose. An individual having high insulin sensitivity effectively processes glucose whereas an individual with low insulin sensitivity does not effectively process glucose.

As used herein, “lipid-lowering” means a reduction in one or more lipids (e.g., LDL, VLDL) in a subject. “Lipid-raising” means an increase in a lipid (e.g., HDL) in a subject. Lipid-lowering or lipid-raising can occur with one or more doses over time.

As used herein, “lipid-lowering therapy” or “lipid lowering agent” means a therapeutic regimen provided to a subject to reduce one or more lipids in a subject. In certain embodiments, a lipid-lowering therapy is provided to reduce one or more of apo(a), CETP, apoB, total cholesterol, LDL-C, VLDL-C, IDL-C, non-HDL-C, triglycerides, small dense LDL particles, and Lp(a) in a subject. Examples of lipid-lowering therapy include, but are not limited to, apoB inhibitors, statins, fibrates and MTP inhibitors.

As used herein, “lipoprotein”, such as VLDL, LDL and HDL, refers to a group of proteins found in the serum, plasma and lymph and are important for lipid transport. The chemical composition of each lipoprotein differs, for example, in that the HDL has a higher proportion of protein versus lipid, whereas the VLDL has a lower proportion of protein versus lipid.

As used herein, “Lp(a)” comprises apo(a) and a LDL like particle containing apoB. The apo(a) is linked to the apoB by a disulfide bond.

As used herein, “low density lipoprotein-cholesterol (LDL-C)” means cholesterol carried in low density lipoprotein particles. Concentration of LDL-C in serum (or plasma) is typically quantified in mg/dL or nmol/L. “Serum LDL-C” and “plasma LDL-C” mean LDL-C in the serum and plasma, respectively.

As used herein, “major risk factors” refers to factors that contribute to a high risk for a particular disease or condition. In certain embodiments, major risk factors for coronary heart disease include, without limitation, cigarette smoking, hypertension, high LDL, low HDL-C, family history of coronary heart disease, age, and other factors disclosed herein.

As used herein, “metabolic disorder” or “metabolic disease” refers to a condition characterized by an alteration or disturbance in metabolic function. “Metabolic” and “metabolism” are terms well known in the art and generally include the whole range of biochemical processes that occur within a living organism. Metabolic disorders include, but are not limited to, hyperglycemia, prediabetes, diabetes (type 1 and type 2), obesity, insulin resistance, metabolic syndrome and dyslipidemia due to type 2 diabetes.

As used herein, “metabolic syndrome” means a condition characterized by a clustering of lipid and non-lipid cardiovascular risk factors of metabolic origin. In certain embodiments, metabolic syndrome is identified by the presence of any 3 of the following factors: waist circumference of greater than 102 cm in men or greater than 88 cm in women; serum triglyceride of at least 150 mg/dL; HDL-C less than 40 mg/dL in men or less than 50 mg/dL in women; blood pressure of at least 130/85 mmHg; and fasting glucose of at least 110 mg/dL. These determinants can be readily measured in clinical practice (JAMA, 2001, 285:2486-2497).

“Parenteral administration” means administration through injection or infusion. Parenteral administration includes subcutaneous administration, intravenous administration, intramuscular administration, intra-arterial administration, intraperitoneal administration, or intracranial administration, e.g., intrathecal or intracerebroventricular administration. Administration can be continuous, chronic, short or intermittent.

As used herein, “pharmaceutical agent” means a substance that provides a therapeutic benefit when administered to an individual. For example, in certain embodiments, an antisense oligonucleotide targeted to apo(a) is a pharmaceutical agent.

As used herein, “pharmaceutical composition” or “composition” means a mixture of substances suitable for administering to an individual. For example, a pharmaceutical composition can comprise one or more active agents and a pharmaceutical carrier e.g., a sterile aqueous solution.

As used herein, “pharmaceutically acceptable derivative” encompasses derivatives of the compounds described herein such as solvates, hydrates, esters, prodrugs, polymorphs, isomers, isotopically labelled variants, pharmaceutically acceptable salts and other derivatives known in the art.

As used herein, “pharmaceutically acceptable salts” means physiologically and pharmaceutically acceptable salts of antisense compounds, i.e., salts that retain the desired biological activity of the parent compound and do not impart undesired toxicological effects thereto. The term “pharmaceutically acceptable salt” or “salt” includes a salt prepared from pharmaceutically acceptable non-toxic acids or bases, including inorganic or organic acids and bases. “Pharmaceutically acceptable salts” of the compounds described herein may be prepared by methods well-known in the art. For a review of pharmaceutically acceptable salts, see Stahl and Wermuth, Handbook of Pharmaceutical Salts: Properties, Selection and Use (Wiley-VCH, Weinheim, Germany, 2002). Sodium salts of antisense oligonucleotides are useful and are well accepted for therapeutic administration to humans. Accordingly, in one embodiment the compounds described herein are in the form of a sodium salt.

As used herein, “portion” means a defined number of contiguous (i.e. linked) nucleobases of a nucleic acid. In certain embodiments, a portion is a defined number of contiguous nucleobases of a target nucleic acid. In certain embodiments, a portion is a defined number of contiguous nucleobases of an antisense compound.

As used herein, “prevent” or “preventing” refers to delaying or forestalling the onset or development of a disease, disorder, or condition for a period of time from minutes to indefinitely. Prevent also means reducing risk of developing a disease, disorder, or condition.

As used herein, “raise” means to increase in amount. For example, to raise plasma HDL levels means to increase the amount of HDL in the plasma.

As used herein, “reduce” means to bring down to a smaller extent, size, amount, or number. For example, to reduce plasma triglyceride levels means to bring down the amount of triglyceride in the plasma.

As used herein, “region” or “target region” is defined as a portion of the target nucleic acid having at least one identifiable structure, function, or characteristic. For example, a target region may encompass a 3′ UTR, a 5′ UTR, an exon, an intron, an exon/intron junction, a coding region, a translation initiation region, translation termination region, or other defined nucleic acid region. The structurally defined regions for apo(a) can be obtained by accession number from sequence databases such as NCBI and such information is incorporated herein by reference. In certain embodiments, a target region may encompass the sequence from a 5′ target site of one target segment within the target region to a 3′ target site of another target segment within the target region.

As used herein, “second agent” or “second therapeutic agent” means an agent that can be used in combination with a “first agent”. A second therapeutic agent can include, but is not limited to, antisense oligonucleotides targeting apo(a) or apoB. A second agent can also include anti-apo(a) antibodies, apo(a) peptide inhibitors, cholesterol lowering agents, lipid lowering agents, glucose lowering agents and anti-inflammatory agents.

As used herein, “segments” are defined as smaller, sub-portions of regions within a nucleic acid. For example, a “target segment” means the sequence of nucleotides of a target nucleic acid to which one or more anti sense compounds is targeted. “5′ target site” refers to the 5′-most nucleotide of a target segment. “3′ target site” refers to the 3 ‘-most nucleotide of a target segment. Alternatively, a “start site” can refer to the 5’-most nucleotide of a target segment and a “stop site” refers to the 3′-most nucleotide of a target segment. A target segment can also begin at the “start site” of one sequence and end at the “stop site” of another sequence.

As used herein, “statin” means an agent that inhibits the activity ofHMG-COA reductase.

As used herein, “subcutaneous administration” means administration just below the skin.

As used herein, “subject” means a human selected for treatment or therapy.

As used herein, “symptom of cardiovascular disease or disorder” means a phenomenon that arises from, accompanies the cardiovascular disease or disorder, and serves as an indication of it. For example, angina; chest pain; shortness of breath; palpitations; weakness; dizziness; nausea; sweating; tachycardia; bradycardia; arrhythmia; atrial fibrillation; swelling in the lower extremities; cyanosis; fatigue; fainting; numbness of the face; numbness of the limbs; claudication or cramping of muscles; bloating of the abdomen; or fever are symptoms of cardiovascular disease or disorder.

As used herein, “targeting” or “targeted” means the process of design and selection of an antisense compound that will specifically hybridize to a target nucleic acid and induce a desired effect.

As used herein, “therapeutically effective amount” means an amount of a pharmaceutical agent that provides a therapeutic benefit to an individual.

As used herein, “therapeutic lifestyle change” means dietary and lifestyle changes intended to lower fat/adipose tissue mass and/or cholesterol. Such change can reduce the risk of developing heart disease, and may include recommendations for dietary intake of total daily calories, total fat, saturated fat, polyunsaturated fat, monounsaturated fat, carbohydrate, protein, cholesterol, insoluble fiber, as well as recommendations for physical activity.

As used herein, “treat” or “treating” refers to administering a compound described herein to effect an alteration or improvement of a disease, disorder, or condition.

As used herein, “prevent” or “preventing” refers inhibit or delay one or more symptoms of a disease, disorder, or condition described herein. For example, in certain embodiments, administration of ISIS 681257 to a subject will prevent one or more symptoms of a cardiovascular disorder, e.g. administration of ISIS 681257 to a subject will inhibit or delay one or more symptoms associated with a cardiovascular disorder.

As used herein, “triglyceride” or “TG” means a lipid or neutral fat consisting of glycerol combined with three fatty acid molecules.

As used herein, “type 2 diabetes,” (also known as “type 2 diabetes mellitus”, “diabetes mellitus, type 2”, “non-insulin-dependent diabetes”, “NIDDM”, “obesity related diabetes”, or “adult-onset diabetes”) is a metabolic disorder that is primarily characterized by insulin resistance, relative insulin deficiency, and hyperglycemia.

### Chemical Nomenclature, Terms, and Conventions

Unless otherwise specified, conventional definitions of terms control and conventional stable atom valences are presumed and achieved in all formulas and groups.

The articles “a” and “an” are used in this disclosure to refer to one or more than one (e.g., to at least one) of the grammatical object of the article. By way of example, “an element” means one element or more than one element.

The term “and/or” is used in this disclosure to mean either “and” or “or” unless indicated otherwise.

### Specific Embodiments

Various embodiments of the invention are described herein. It will be recognized that features specified in each embodiment may be combined with other specified features of other embodiments to provide further embodiments.

In a first aspect, the present disclosure relates to a method of reducing the risk of a cardiovascular event in a patient who has established cardiovascular disease comprising, administering a unit dose comprising from about 75 mg to about 85 mg of the compound ISIS 681257, or a salt thereof, by subcutaneous injection to the patient once a month, wherein said patient has a plasma Lp(a) concentration greater than or equal to 70 mg/dL prior to the time of the first administration of the compound.

In some embodiments, the cardiovascular event is selected from a major adverse cardiovascular event (MACE), all cause death (death from any cause), coronary heart disease (CHD) death, acute myocardial infarction (AMI) death, heart failure (HF) death, death caused by the immediate complications of a cardiac procedure, and urgent lower limb re-vascularization or amputation for ischemia.

In some embodiments, the major adverse cardiovascular event (MACE) is selected from cardiovascular (CV) death, non-fatal myocardial infarction, non-fatal stroke, and urgent coronary re-vascularization requiring hospitalization. In another embodiment, the major adverse cardiovascular event (MACE) is cardiovascular (CV) death. In yet another embodiment, the major adverse cardiovascular event (MACE) is non-fatal myocardial infarction. In another embodiment, the major adverse cardiovascular event (MACE) is non-fatal stroke. In yet another embodiment, the major adverse cardiovascular event (MACE) is urgent coronary re-vascularization requiring hospitalization.

In some embodiments, the cardiovascular event is selected from all cause death (death from any cause), coronary heart disease (CHD) death, acute myocardial infarction (AMI) death, heart failure (HF) death, death caused by the immediate complications of a cardiac procedure, and urgent lower limb re-vascularization or amputation for ischemia.

In one embodiment, the cardiovascular event is all cause death (death from any cause).

In another embodiment, the cardiovascular event is coronary heart disease (CHD) death. In one embodiment, the coronary heart disease (CHD) death comprises acute myocardial infarction (AMI) death, heart failure (HF) death, and death caused by the immediate complications of a cardiac procedure.

In another embodiment, the cardiovascular event is urgent lower limb re-vascularization or amputation for ischemia.

In some embodiments, the patient who has established cardiovascular disease is a patient having at least one of the following (i) a history of spontaneous myocardial infarction, (i) a history of ischemic stroke, and (iii) clinically significant symptomatic peripheral artery disease.

In one embodiment, the history of spontaneous myocardial infarction occurred ≥3 months and ≤10 years prior to the time of the first administration of the compound.

In one embodiment, the history of ischemic stroke occurred ≥3 months and ≤10 years prior to the time of the first administration of the compound.

In one embodiment, the history of ischemic stroke is an acute episode of focal cerebral, spinal, or retinal dysfunction caused by infarction of central nervous system tissue.

In one embodiment, the clinically significant symptomatic peripheral artery disease is evidenced by intermittent claudication with at least one of (i) an ankle-brachial index ≤0.90; and (ii) lower limb amputation or re-vascularization due to lower limb ischemia.

In some embodiments, the patient has a plasma Lp(a) concentration ≥90 mg/dL prior to the time of the first administration of the compound.

In some embodiments, the compound is formulated in a sterile liquid and wherein each unit dose of the compound does not comprise more than 1 mL of the sterile liquid.

In one embodiment, each unit dose of the compound does not comprise more than 0.8 mL of the sterile liquid. In another embodiment, each unit dose of the compound does not comprise more than 0.5 mL of the sterile liquid. In yet another embodiment, each unit dose of the compound does not comprise more than 0.4 mL of the sterile liquid. In another embodiment, each unit dose of the compound does not comprise not more than 0.25 mL of the sterile liquid. In yet another embodiment, each unit dose of the compound does not comprise not more than 0.2 mL of the sterile liquid.

In one embodiment, the sterile liquid is water. In another embodiment, the sterile liquid is water with a sodium phosphate buffer. In yet another embodiment, the sterile liquid is water with a sodium phosphate buffer and sodium chloride.

In some embodiments, the mean/median plasma Lp(a) concentration in the patient is reduced by at least 50%, when the plasma Lp(a) concentration in the patient is measured at the start and end of the dosing period. In another embodiment, the mean/median plasma Lp(a) concentration in the patient is reduced by at least 60%, when the plasma Lp(a) concentration in the patient is measured at the start and end of the dosing period. In yet another embodiment, the mean/median plasma Lp(a) concentration in the patient is reduced by at least 70%, when the plasma Lp(a) concentration in the patient is measured at the start and end of the dosing period. In another embodiment, the mean/median plasma Lp(a) concentration in the patient is reduced by at least 75%, when the plasma Lp(a) concentration in the patient is measured at the start and end of the dosing period.

In some embodiments, the mean/median plasma Lp(a) concentration in the patient is reduced by about 30%, about 35%, about 40%, about 45%, about 50%, about 55%, about 60%, about 65%, about 70%, about 75%, about 80%, about 85%, or about 90%, when the plasma Lp(a) concentration in the patient is measured at the start and end of the dosing period.

In some embodiments, the mean/median plasma Lp(a) concentration in the patient is reduced by about 40% to about 50%, when the plasma Lp(a) concentration in the patient is measured at the start and end of the dosing period. In another embodiment, the mean/median plasma Lp(a) concentration in the patient is reduced by about 45% to about 55%, when the plasma Lp(a) concentration in the patient is measured at the start and end of the dosing period. In another embodiment, the mean/median plasma Lp(a) concentration in the patient is reduced by about 50% to about 60%, when the plasma Lp(a) concentration in the patient is measured at the start and end of the dosing period. In yet another embodiment, the mean/median plasma Lp(a) concentration in the patient is reduced by about 55% to about 65%, when the plasma Lp(a) concentration in the patient is measured at the start and end of the dosing period. In yet another embodiment, the mean/median plasma Lp(a) concentration in the patient is reduced by about 60% to about 70%, when the plasma Lp(a) concentration in the patient is measured at the start and end of the dosing period. In another embodiment, the mean/median plasma Lp(a) concentration in the patient is reduced by about 65% to about 75%, when the plasma Lp(a) concentration in the patient is measured at the start and end of the dosing period. In another embodiment, the mean/median plasma Lp(a) concentration in the patient is reduced by about 70% to about 85%, when the plasma Lp(a) concentration in the patient is measured at the start and end of the dosing period.

In some embodiments, the overall risk of the patient to experience a major adverse cardiovascular event (MACE) is reduced by a statistically significant amount at the end of the dosing period in comparison to patients who were not administered the compound.

In other embodiments, the overall risk of the patient to experience one of the following events is reduced by a statistically significant amount at the end of the dosing period in comparison to patients who were not administered the compound: (i) the composite of cardiovascular (CV) death, non-fatal MI and non-fatal stroke; (ii) the composite of coronary heart disease (CHD) death, non-fatal MI and urgent coronary re-vascularization requiring hospitalization; (iii) the composite of coronary heart disease (CHD) death, non-fatal MI, urgent coronary re-vascularization requiring hospitalization and urgent lower limb re-vascularization or amputation for ischemia; and (iv) the rate of all cause death.

In one embodiment thereof, the overall risk of the patient to experience the following event is reduced by a statistically significant amount at the end of the dosing period in comparison to patients who were not administered the compound: the composite of cardiovascular (CV) death, non-fatal MI and non-fatal stroke.

In one embodiment thereof, the overall risk of the patient to experience the following event is reduced by a statistically significant amount at the end of the dosing period in comparison to patients who were not administered the compound: the composite of coronary heart disease (CHD) death, non-fatal MI and urgent coronary re-vascularization requiring hospitalization.

In one embodiment thereof, the overall risk of the patient to experience the following event is reduced by a statistically significant amount at the end of the dosing period in comparison to patients who were not administered the compound: the composite of coronary heart disease (CHD) death, non-fatal MI, urgent coronary re-vascularization requiring hospitalization and urgent lower limb re-vascularization or amputation for ischemia.

In one embodiment thereof, the overall risk of the patient to experience the following event is reduced by a statistically significant amount at the end of the dosing period in comparison to patients who were not administered the compound: the rate of all cause death.

In other embodiments, the overall risk of the patient to experience one of the following events is reduced by a statistically significant amount at the end of the dosing period in comparison to patients who were not administered the compound, and wherein the patient has a plasma Lp(a) concentration ≥90 mg/dL prior to the time of the first administration of the compound: (i) the composite of all-cause mortality, non-fatal MI and non-fatal stroke; (ii) the composite of total vascular events: CV death, non-fatal MI, non-fatal stroke, urgent coronary re-vascularization requiring hospitalization and urgent lower limb re-vascularization or amputation for ischemia; (iii) the composite of all-cause mortality, non-fatal MI, non-fatal stroke and urgent coronary re-vascularization requiring hospitalization; (iv) the composite of fatal and non-fatal stroke, (v) the rate of major adverse limb events (MALE) in patients with history of peripheral artery disease (PAD), (vi) the rate of hospitalization for unstable angina, and (vii) the rate of hospitalizations for heart failure.

In one embodiment thereof, the overall risk of the patient having a plasma Lp(a) concentration ≥90 mg/dL prior to the time of the first administration of the compound to experience the following event is reduced by a statistically significant amount at the end of the dosing period in comparison to patients who were not administered the compound: the composite of all-cause mortality, non-fatal MI and non-fatal stroke.

In one embodiment thereof, the overall risk of the patient having a plasma Lp(a) concentration ≥90 mg/dL prior to the time of the first administration of the compound to experience the following event is reduced by a statistically significant amount at the end of the dosing period in comparison to patients who were not administered the compound: the composite of total vascular events: CV death, non-fatal MI, non-fatal stroke, urgent coronary re-vascularization requiring hospitalization and urgent lower limb re-vascularization or amputation for ischemia.

In one embodiment thereof, the overall risk of the patient having a plasma Lp(a) concentration ≥90 mg/dL prior to the time of the first administration of the compound to experience the following event is reduced by a statistically significant amount at the end of the dosing period in comparison to patients who were not administered the compound: the composite of all-cause mortality, non-fatal MI, non-fatal stroke and urgent coronary re-vascularization requiring hospitalization.

In one embodiment thereof, the overall risk of the patient having a plasma Lp(a) concentration ≥90 mg/dL prior to the time of the first administration of the compound to experience the following event is reduced by a statistically significant amount at the end of the dosing period in comparison to patients who were not administered the compound: the composite of fatal and non-fatal stroke.

In one embodiment thereof, the overall risk of the patient having a plasma Lp(a) concentration ≥90 mg/dL prior to the time of the first administration of the compound to experience the following event is reduced by a statistically significant amount at the end of the dosing period in comparison to patients who were not administered the compound: the rate of major adverse limb events (MALE) in patients with history of peripheral artery disease (PAD).

In one embodiment thereof, the overall risk of the patient having a plasma Lp(a) concentration ≥90 mg/dL prior to the time of the first administration of the compound to experience the following event is reduced by a statistically significant amount at the end of the dosing period in comparison to patients who were not administered the compound: the rate of hospitalization for unstable angina.

In one embodiment thereof, the overall risk of the patient having a plasma Lp(a) concentration ≥90 mg/dL prior to the time of the first administration of the compound to experience the following event is reduced by a statistically significant amount at the end of the dosing period in comparison to patients who were not administered the compound: the rate of hospitalizations for heart failure.

In one embodiment of any one of the above embodiments concerning the risk reduction rate, the relative risk reduction rate (i.e., the statistically significant relative amount by which the overall risk is reduced) is at least 15% for any one of the events for a patient having a plasma Lp(a) concentration greater than or equal to 70 mg/dL prior to the time of the first administration of the compound. In another embodiment, the relative risk reduction rate for any one of the events is (i) at least 15%, preferably at least 20%, more preferably at least 25%, for a patient having a plasma Lp(a) concentration greater than or equal to 70 mg/dL prior to the time of the first administration of the compound; (ii) at least 20%, preferably at least 25%, more preferably at least 30%, for a patient having a plasma Lp(a) concentration greater than or equal to 90 mg/dL prior to the time of the first administration of the compound.

In one embodiment of any one of the above embodiments concerning the risk reduction rate, the relative risk reduction rate (i.e., the statistically significant relative amount by which the overall risk is reduced) is about 10%, about 15%, about 20%, about 25%, about 30%, about 35%, or about 40% for any one of the events for a patient having a plasma Lp(a) concentration greater than or equal to 70 mg/dL prior to the time of the first administration of the compound.

In one embodiment of any one of the above embodiments concerning the risk reduction rate, the relative risk reduction rate (i.e., the statistically significant relative amount by which the overall risk is reduced) is about 10% to about 20% for any one of the events for a patient having a plasma Lp(a) concentration greater than or equal to 70 mg/dL prior to the time of the first administration of the compound. In another embodiment, the relative risk reduction rate for any one of the events is about 15% to about 25% for any one of the events for a patient having a plasma Lp(a) concentration greater than or equal to 70 mg/dL prior to the time of the first administration of the compound. In another embodiment, the relative risk reduction rate for any one of the events is about 20% to about 30%, for a patient having a plasma Lp(a) concentration greater than or equal to 70 mg/dL prior to the time of the first administration of the compound. In another embodiment, the relative risk reduction rate for any one of the events is about 25% to about 35%, for a patient having a plasma Lp(a) concentration greater than or equal to 70 mg/dL prior to the time of the first administration of the compound. In another embodiment, the relative risk reduction rate for any one of the events is about 30% to about 40%, for a patient having a plasma Lp(a) concentration greater than or equal to 70 mg/dL prior to the time of the first administration of the compound.

In one embodiment of any one of the above embodiments concerning the risk reduction rate, the relative risk reduction rate (i.e., the statistically significant relative amount by which the overall risk is reduced) is about 10% to about 15% for any one of the events for a patient having a plasma Lp(a) concentration greater than or equal to 70 mg/dL prior to the time of the first administration of the compound. In another embodiment, the relative risk reduction rate for any one of the events is about 15% to about 20% for any one of the events for a patient having a plasma Lp(a) concentration greater than or equal to 70 mg/dL prior to the time of the first administration of the compound. In another embodiment, the relative risk reduction rate for any one of the events is about 20% to about 25%, for a patient having a plasma Lp(a) concentration greater than or equal to 70 mg/dL prior to the time of the first administration of the compound. In another embodiment, the relative risk reduction rate for any one of the events is about 25% to about 30%, for a patient having a plasma Lp(a) concentration greater than or equal to 70 mg/dL prior to the time of the first administration of the compound. In another embodiment, the relative risk reduction rate for any one of the events is about 30% to about 35%, for a patient having a plasma Lp(a) concentration greater than or equal to 70 mg/dL prior to the time of the first administration of the compound. In another embodiment, the relative risk reduction rate for any one of the events is about 35% to about 40%, for a patient having a plasma Lp(a) concentration greater than or equal to 70 mg/dL prior to the time of the first administration of the compound.

In one embodiment of any one of the above embodiments concerning the risk reduction rate, the relative risk reduction rate (i.e., the statistically significant relative amount by which the overall risk is reduced) is about 10% for any one of the events for a patient having a plasma Lp(a) concentration greater than or equal to 70 mg/dL prior to the time of the first administration of the compound. In another embodiment, the relative risk reduction rate for any one of the events is about 15% for any one of the events for a patient having a plasma Lp(a) concentration greater than or equal to 70 mg/dL prior to the time of the first administration of the compound. In another embodiment, the relative risk reduction rate for any one of the events is about 20% for any one of the events for a patient having a plasma Lp(a) concentration greater than or equal to 70 mg/dL prior to the time of the first administration of the compound. In another embodiment, the relative risk reduction rate for any one of the events is about 25%, for a patient having a plasma Lp(a) concentration greater than or equal to 70 mg/dL prior to the time of the first administration of the compound. In another embodiment, the relative risk reduction rate for any one of the events is about 30%, for a patient having a plasma Lp(a) concentration greater than or equal to 70 mg/dL prior to the time of the first administration of the compound. In another embodiment, the relative risk reduction rate for any one of the events is about 35% for any one of the events for a patient having a plasma Lp(a) concentration greater than or equal to 70 mg/dL prior to the time of the first administration of the compound.

In one embodiment of any one of the above embodiments concerning the risk reduction rate, the relative risk reduction rate (i.e., the statistically significant relative amount by which the overall risk is reduced) is at least about 10% for any one of the events for a patient having a plasma Lp(a) concentration greater than or equal to 70 mg/dL prior to the time of the first administration of the compound. In another embodiment, the relative risk reduction rate for any one of the events is at least about 15% for any one of the events for a patient having a plasma Lp(a) concentration greater than or equal to 70 mg/dL prior to the time of the first administration of the compound. In another embodiment, the relative risk reduction rate for any one of the events is at least about 20% for any one of the events for a patient having a plasma Lp(a) concentration greater than or equal to 70 mg/dL prior to the time of the first administration of the compound. In another embodiment, the relative risk reduction rate for any one of the events is at least about 25%, for a patient having a plasma Lp(a) concentration greater than or equal to 70 mg/dL prior to the time of the first administration of the compound. In another embodiment, the relative risk reduction rate for any one of the events is at least about 30%, for a patient having a plasma Lp(a) concentration greater than or equal to 70 mg/dL prior to the time of the first administration of the compound. In another embodiment, the relative risk reduction rate for any one of the events is at least about 35%, for a patient having a plasma Lp(a) concentration greater than or equal to 70 mg/dL prior to the time of the first administration of the compound.

In one embodiment of any one of the above embodiments concerning the risk reduction rate, the relative risk reduction rate (i.e., the statistically significant relative amount by which the overall risk is reduced) is at least 10% for any one of the events for a patient having a plasma Lp(a) concentration greater than or equal to 70 mg/dL prior to the time of the first administration of the compound. In another embodiment, the relative risk reduction rate for any one of the events is at least 15% for any one of the events for a patient having a plasma Lp(a) concentration greater than or equal to 70 mg/dL prior to the time of the first administration of the compound. In another embodiment, the relative risk reduction rate for any one of the events is at least 20% for any one of the events for a patient having a plasma Lp(a) concentration greater than or equal to 70 mg/dL prior to the time of the first administration of the compound. In another embodiment, the relative risk reduction rate for any one of the events is at least 25%, for a patient having a plasma Lp(a) concentration greater than or equal to 70 mg/dL prior to the time of the first administration of the compound. In another embodiment, the relative risk reduction rate for any one of the events is at least 30%, for a patient having a plasma Lp(a) concentration greater than or equal to 70 mg/dL prior to the time of the first administration of the compound. In another embodiment, the relative risk reduction rate for any one of the events is at least 35%, for a patient having a plasma Lp(a) concentration greater than or equal to 70 mg/dL prior to the time of the first administration of the compound.

In one embodiment of any one of the above embodiments concerning the risk reduction rate, the relative risk reduction rate is about 10%, about 15%, about 20%, about 25%, about 30%, about 35%, or about 40% for any one of the events for a patient having a plasma Lp(a) concentration greater than or equal to 90 mg/dL prior to the time of the first administration of the compound.

In one embodiment of any one of the above embodiments concerning the risk reduction rate, the relative risk reduction rate (i.e., the statistically significant relative amount by which the overall risk is reduced) is about 10 to about 20% for any one of the events for a patient having a plasma Lp(a) concentration greater than or equal to 90 mg/dL prior to the time of the first administration of the compound. In yet another embodiment, the relative risk reduction rate (i.e., the statistically significant relative amount by which the overall risk is reduced) is about 15 to about 25% for a patient having a plasma Lp(a) concentration greater than or equal to 90 mg/dL prior to the time of the first administration of the compound. In another embodiment, the relative risk reduction rate (i.e., the statistically significant relative amount by which the overall risk is reduced) is about 20% to about 30% for a patient having a plasma Lp(a) concentration greater than or equal to 90 mg/dL prior to the time of the first administration of the compound. In yet another embodiment, the relative risk reduction rate (i.e., the statistically significant relative amount by which the overall risk is reduced) is about 25% to about 35% for a patient having a plasma Lp(a) concentration greater than or equal to 90 mg/dL prior to the time of the first administration of the compound. In another embodiment, the relative risk reduction rate (i.e., the statistically significant relative amount by which the overall risk is reduced) is about 30% to about 40% for a patient having a plasma Lp(a) concentration greater than or equal to 90 mg/dL prior to the time of the first administration of the compound.

In one embodiment of any one of the above embodiments concerning the risk reduction rate, the relative risk reduction rate (i.e., the statistically significant relative amount by which the overall risk is reduced) is about 10 to about 15% for any one of the events for a patient having a plasma Lp(a) concentration greater than or equal to 90 mg/dL prior to the time of the first administration of the compound. In yet another embodiment, the relative risk reduction rate (i.e., the statistically significant relative amount by which the overall risk is reduced) is about 15 to about 20% for a patient having a plasma Lp(a) concentration greater than or equal to 90 mg/dl prior to the time of the first administration of the compound. In another embodiment, the relative risk reduction rate (i.e., the statistically significant relative amount by which the overall risk is reduced) is about 20% to about 25% for a patient having a plasma Lp(a) concentration greater than or equal to 90 mg/dL prior to the time of the first administration of the compound. In yet another embodiment, the relative risk reduction rate (i.e., the statistically significant relative amount by which the overall risk is reduced) is about 25% to about 30% for a patient having a plasma Lp(a) concentration greater than or equal to 90 mg/dL prior to the time of the first administration of the compound. In another embodiment, the relative risk reduction rate (i.e., the statistically significant relative amount by which the overall risk is reduced) is about 30% to about 35% for a patient having a plasma Lp(a) concentration greater than or equal to 90 mg/dL prior to the time of the first administration of the compound. In another embodiment, the relative risk reduction rate (i.e., the statistically significant relative amount by which the overall risk is reduced) is about 35% to about 40% for a patient having a plasma Lp(a) concentration greater than or equal to 90 mg/dL prior to the time of the first administration of the compound.

In one embodiment of any one of the above embodiments concerning the risk reduction rate, the relative risk reduction rate (i.e., the statistically significant relative amount by which the overall risk is reduced) is about 15% for any one of the events for a patient having a plasma Lp(a) concentration greater than or equal to 90 mg/dL prior to the time of the first administration of the compound. In yet another embodiment, the relative risk reduction rate (i.e., the statistically significant relative amount by which the overall risk is reduced) is about 20% for a patient having a plasma Lp(a) concentration greater than or equal to 90 mg/dL prior to the time of the first administration of the compound. In another embodiment, the relative risk reduction rate (i.e., the statistically significant relative amount by which the overall risk is reduced) is about 25% for a patient having a plasma Lp(a) concentration greater than or equal to 90 mg/dL prior to the time of the first administration of the compound. In yet another embodiment, the relative risk reduction rate (i.e., the statistically significant relative amount by which the overall risk is reduced) is about 30% for a patient having a plasma Lp(a) concentration greater than or equal to 90 mg/dL prior to the time of the first administration of the compound. In another embodiment, the relative risk reduction rate (i.e., the statistically significant relative amount by which the overall risk is reduced) is about 35% for a patient having a plasma Lp(a) concentration greater than or equal to 90 mg/dL prior to the time of the first administration of the compound.

In one embodiment of any one of the above embodiments concerning the risk reduction rate, the relative risk reduction rate (i.e., the statistically significant relative amount by which the overall risk is reduced) is at least about 15% for any one of the events for a patient having a plasma Lp(a) concentration greater than or equal to 90 mg/dL prior to the time of the first administration of the compound. In yet another embodiment, the relative risk reduction rate (i.e., the statistically significant relative amount by which the overall risk is reduced) is at least about 20% for a patient having a plasma Lp(a) concentration greater than or equal to 90 mg/dL prior to the time of the first administration of the compound. In another embodiment, the relative risk reduction rate (i.e., the statistically significant relative amount by which the overall risk is reduced) is at least about 25% for a patient having a plasma Lp(a) concentration greater than or equal to 90 mg/dL prior to the time of the first administration of the compound. In yet another embodiment, the relative risk reduction rate (i.e., the statistically significant relative amount by which the overall risk is reduced) is at least about 30% for a patient having a plasma Lp(a) concentration greater than or equal to 90 mg/dL prior to the time of the first administration of the compound. In another embodiment, the relative risk reduction rate (i.e., the statistically significant relative amount by which the overall risk is reduced) is at least about 35% for a patient having a plasma Lp(a) concentration greater than or equal to 90 mg/dL prior to the time of the first administration of the compound.

In one embodiment of any one of the above embodiments concerning the risk reduction rate, the relative risk reduction rate (i.e., the statistically significant relative amount by which the overall risk is reduced) is at least 15% for any one of the events for a patient having a plasma Lp(a) concentration greater than or equal to 90 mg/dL prior to the time of the first administration of the compound. In yet another embodiment, the relative risk reduction rate (i.e., the statistically significant relative amount by which the overall risk is reduced) is at least 20% for a patient having a plasma Lp(a) concentration greater than or equal to 90 mg/dL prior to the time of the first administration of the compound. In another embodiment, the relative risk reduction rate (i.e., the statistically significant relative amount by which the overall risk is reduced) is at least 25% for a patient having a plasma Lp(a) concentration greater than or equal to 90 mg/dL prior to the time of the first administration of the compound. In yet another embodiment, the relative risk reduction rate (i.e., the statistically significant relative amount by which the overall risk is reduced) is at least 30% for a patient having a plasma Lp(a) concentration greater than or equal to 90 mg/dL prior to the time of the first administration of the compound. In another embodiment, the relative risk reduction rate (i.e., the statistically significant relative amount by which the overall risk is reduced) is at least 35% for a patient having a plasma Lp(a) concentration greater than or equal to 90 mg/dL prior to the time of the first administration of the compound.

In one embodiment of any one of the above embodiments concerning the risk reduction rate, the absolute risk reduction rate (i.e., the statistically significant absolute amount by which the overall risk is reduced) for any one of the events is (i) at least 2.0%, preferably at least 2.5%, for a patient having a plasma Lp(a) concentration greater than or equal to 70 mg/dL prior to the time of the first administration of the compound; (ii) at least 3.0%, preferably at least 3.5%, for a patient having a plasma Lp(a) concentration greater than or equal to 90 mg/dL prior to the time of the first administration of the compound.

In one embodiment of any one of the above embodiments concerning the risk reduction rate, the absolute risk reduction rate (i.e., the statistically significant absolute amount by which the overall risk is reduced) for any one of the events is about 1.5%, about 1.8%, about 2.0%, about 2.2%, about 2.5%, about 2.8%, about 3.0%, about 3.2%, about 3.5%, about 3.8%, about 4.0%, about 4.2%, about 4.5%, about 4.8%, or about 5.0% for a patient having a plasma Lp(a) concentration greater than or equal to 70 mg/dL prior to the time of the first administration of the compound.

In one embodiment of any one of the above embodiments concerning the risk reduction rate, the absolute risk reduction rate (i.e., the statistically significant absolute amount by which the overall risk is reduced) for any one of the events is at least about 1.5%, about 1.8%, about 2.0%, about 2.2%, about 2.5%, about 2.8%, about 3.0%, about 3.2%, about 3.5%, about 3.8%, about 4.0%, about 4.2%, about 4.5%, about 4.8%, or about 5.0% for a patient having a plasma Lp(a) concentration greater than or equal to 70 mg/dL prior to the time of the first administration of the compound.

In one embodiment of any one of the above embodiments concerning the risk reduction rate, the absolute risk reduction rate (i.e., the statistically significant absolute amount by which the overall risk is reduced) for any one of the events is about 1.5% to about 2.5% for a patient having a plasma Lp(a) concentration greater than or equal to 70 mg/dL prior to the time of the first administration of the compound. In another embodiment, the absolute risk reduction rate (i.e., the statistically significant absolute amount by which the overall risk is reduced) for any one of the events is about 2.0% to about 3.0% for a patient having a plasma Lp(a) concentration greater than or equal to 70 mg/dL prior to the time of the first administration of the compound. In yet another embodiment, the absolute risk reduction rate (i.e., the statistically significant absolute amount by which the overall risk is reduced) for any one of the events is about 2.5% to about 3.5%, for a patient having a plasma Lp(a) concentration greater than or equal to 70 mg/dL prior to the time of the first administration of the compound. In another embodiment, the absolute risk reduction rate (i.e., the statistically significant absolute amount by which the overall risk is reduced) for any one of the events is about 3.0% to about 4.0% for a patient having a plasma Lp(a) concentration greater than or equal to 70 mg/dL prior to the time of the first administration of the compound. In another embodiment, the absolute risk reduction rate (i.e., the statistically significant absolute amount by which the overall risk is reduced) for any one of the events is about 3.5% to about 4.5% for a patient having a plasma Lp(a) concentration greater than or equal to 70 mg/dL prior to the time of the first administration of the compound. In yet another embodiment, the absolute risk reduction rate (i.e., the statistically significant absolute amount by which the overall risk is reduced) for any one of the events is about 4.0% to about 5.0%, for a patient having a plasma Lp(a) concentration greater than or equal to 70 mg/dL prior to the time of the first administration of the compound.

In one embodiment of any one of the above embodiments concerning the risk reduction rate, the absolute risk reduction rate (i.e., the statistically significant absolute amount by which the overall risk is reduced) for any one of the events is about 1.5% to about 2.0% for a patient having a plasma Lp(a) concentration greater than or equal to 70 mg/dL prior to the time of the first administration of the compound. In another embodiment, the absolute risk reduction rate (i.e., the statistically significant absolute amount by which the overall risk is reduced) for any one of the events is about 2.0% to about 2.5% for a patient having a plasma Lp(a) concentration greater than or equal to 70 mg/dL prior to the time of the first administration of the compound. In yet another embodiment, the absolute risk reduction rate (i.e., the statistically significant absolute amount by which the overall risk is reduced) for any one of the events is about 2.5% to about 3.0%, for a patient having a plasma Lp(a) concentration greater than or equal to 70 mg/dL prior to the time of the first administration of the compound. In another embodiment, the absolute risk reduction rate (i.e., the statistically significant absolute amount by which the overall risk is reduced) for any one of the events is about 3.0% to about 3.5% for a patient having a plasma Lp(a) concentration greater than or equal to 70 mg/dL prior to the time of the first administration of the compound. In another embodiment, the absolute risk reduction rate (i.e., the statistically significant absolute amount by which the overall risk is reduced) for any one of the events is about 3.5% to about 4.0% for a patient having a plasma Lp(a) concentration greater than or equal to 70 mg/dL prior to the time of the first administration of the compound. In yet another embodiment, the absolute risk reduction rate (i.e., the statistically significant absolute amount by which the overall risk is reduced) for any one of the events is about 4.0% to about 4.5%, for a patient having a plasma Lp(a) concentration greater than or equal to 70 mg/dL prior to the time of the first administration of the compound. In another embodiment, the absolute risk reduction rate (i.e., the statistically significant absolute amount by which the overall risk is reduced) for any one of the events is about 4.5% to about 5.0%, for a patient having a plasma Lp(a) concentration greater than or equal to 70 mg/dL prior to the time of the first administration of the compound.

In one embodiment of any one of the above embodiments concerning the risk reduction rate, the absolute risk reduction rate (i.e., the statistically significant absolute amount by which the overall risk is reduced) for any one of the events is about 1.8% for a patient having a plasma Lp(a) concentration greater than or equal to 70 mg/dL prior to the time of the first administration of the compound. In another embodiment, the absolute risk reduction rate (i.e., the statistically significant absolute amount by which the overall risk is reduced) for any one of the events is about 2.0% for a patient having a plasma Lp(a) concentration greater than or equal to 70 mg/dL prior to the time of the first administration of the compound. In yet another embodiment, the absolute risk reduction rate (i.e., the statistically significant absolute amount by which the overall risk is reduced) for any one of the events is about 2.5%, for a patient having a plasma Lp(a) concentration greater than or equal to 70 mg/dL prior to the time of the first administration of the compound. In another embodiment, the absolute risk reduction rate (i.e., the statistically significant absolute amount by which the overall risk is reduced) for any one of the events is about 3.0% for a patient having a plasma Lp(a) concentration greater than or equal to 70 mg/dL prior to the time of the first administration of the compound. In another embodiment, the absolute risk reduction rate (i.e., the statistically significant absolute amount by which the overall risk is reduced) for any one of the events is about 3.5% for a patient having a plasma Lp(a) concentration greater than or equal to 70 mg/dL prior to the time of the first administration of the compound. In yet another embodiment, the absolute risk reduction rate (i.e., the statistically significant absolute amount by which the overall risk is reduced) for any one of the events is about 4.5%, for a patient having a plasma Lp(a) concentration greater than or equal to 70 mg/dL prior to the time of the first administration of the compound.

In one embodiment of any one of the above embodiments concerning the risk reduction rate, the absolute risk reduction rate (i.e., the statistically significant absolute amount by which the overall risk is reduced) for any one of the events is at least about 1.8% for a patient having a plasma Lp(a) concentration greater than or equal to 70 mg/dL prior to the time of the first administration of the compound. In another embodiment, the absolute risk reduction rate (i.e., the statistically significant absolute amount by which the overall risk is reduced) for any one of the events is at least about 2.0% for a patient having a plasma Lp(a) concentration greater than or equal to 70 mg/dL prior to the time of the first administration of the compound. In yet another embodiment, the absolute risk reduction rate (i.e., the statistically significant absolute amount by which the overall risk is reduced) for any one of the events is at least about 2.5%, for a patient having a plasma Lp(a) concentration greater than or equal to 70 mg/dL prior to the time of the first administration of the compound. In another embodiment, the absolute risk reduction rate (i.e., the statistically significant absolute amount by which the overall risk is reduced) for any one of the events is at least about 3.0% for a patient having a plasma Lp(a) concentration greater than or equal to 70 mg/dL prior to the time of the first administration of the compound. In another embodiment, the absolute risk reduction rate (i.e., the statistically significant absolute amount by which the overall risk is reduced) for any one of the events is at least about 3.5% for a patient having a plasma Lp(a) concentration greater than or equal to 70 mg/dL prior to the time of the first administration of the compound. In yet another embodiment, the absolute risk reduction rate (i.e., the statistically significant absolute amount by which the overall risk is reduced) for any one of the events is at least about 4.5%, for a patient having a plasma Lp(a) concentration greater than or equal to 70 mg/dL prior to the time of the first administration of the compound.

In one embodiment of any one of the above embodiments concerning the risk reduction rate, the absolute risk reduction rate (i.e., the statistically significant absolute amount by which the overall risk is reduced) for any one of the events is at least 1.8% for a patient having a plasma Lp(a) concentration greater than or equal to 70 mg/dL prior to the time of the first administration of the compound. In another embodiment, the absolute risk reduction rate (i.e., the statistically significant absolute amount by which the overall risk is reduced) for any one of the events is at least 2.0% for a patient having a plasma Lp(a) concentration greater than or equal to 70 mg/dL prior to the time of the first administration of the compound. In yet another embodiment, the absolute risk reduction rate (i.e., the statistically significant absolute amount by which the overall risk is reduced) for any one of the events is at least 2.5%, for a patient having a plasma Lp(a) concentration greater than or equal to 70 mg/dL prior to the time of the first administration of the compound. In another embodiment, the absolute risk reduction rate (i.e., the statistically significant absolute amount by which the overall risk is reduced) for any one of the events is at least 3.0% for a patient having a plasma Lp(a) concentration greater than or equal to 70 mg/dL prior to the time of the first administration of the compound. In another embodiment, the absolute risk reduction rate (i.e., the statistically significant absolute amount by which the overall risk is reduced) for any one of the events is at least 3.5% for a patient having a plasma Lp(a) concentration greater than or equal to 70 mg/dL prior to the time of the first administration of the compound. In yet another embodiment, the absolute risk reduction rate (i.e., the statistically significant absolute amount by which the overall risk is reduced) for any one of the events is at least 4.5%, for a patient having a plasma Lp(a) concentration greater than or equal to 70 mg/dL prior to the time of the first administration of the compound.

In one embodiment of any one of the above embodiments concerning the risk reduction rate, the absolute risk reduction rate (i.e., the statistically significant absolute amount by which the overall risk is reduced) for any one of the events is at least about 2.8%, for a patient having a plasma Lp(a) concentration greater than or equal to 90 mg/dL prior to the time of the first administration of the compound. In yet another embodiment, the absolute risk reduction rate (i.e., the statistically significant absolute amount by which the overall risk is reduced) for any one of the events is at least about 3.0%, for a patient having a plasma Lp(a) concentration greater than or equal to 90 mg/dL prior to the time of the first administration of the compound. In another embodiment, the absolute risk reduction rate (i.e., the statistically significant absolute amount by which the overall risk is reduced) for any one of the events is at least about 3.5%, for a patient having a plasma Lp(a) concentration greater than or equal to 90 mg/dL prior to the time of the first administration of the compound. In yet another embodiment, the absolute risk reduction rate (i.e., the statistically significant absolute amount by which the overall risk is reduced) for any one of the events is at least about 4.0%, for a patient having a plasma Lp(a) concentration greater than or equal to 90 mg/dL prior to the time of the first administration of the compound. In another embodiment, the absolute risk reduction rate (i.e., the statistically significant absolute amount by which the overall risk is reduced) for any one of the events is at least about 4.5%, for a patient having a plasma Lp(a) concentration greater than or equal to 90 mg/dL prior to the time of the first administration of the compound.

In one embodiment of any one of the above embodiments concerning the risk reduction rate, the absolute risk reduction rate (i.e., the statistically significant absolute amount by which the overall risk is reduced) for any one of the events is at least 2.8%, for a patient having a plasma Lp(a) concentration greater than or equal to 90 mg/dL prior to the time of the first administration of the compound. In yet another embodiment, the absolute risk reduction rate (i.e., the statistically significant absolute amount by which the overall risk is reduced) for any one of the events is at least 3.0%, for a patient having a plasma Lp(a) concentration greater than or equal to 90 mg/dL prior to the time of the first administration of the compound. In another embodiment, the absolute risk reduction rate (i.e., the statistically significant absolute amount by which the overall risk is reduced) for any one of the events is at least 3.5%, for a patient having a plasma Lp(a) concentration greater than or equal to 90 mg/dL prior to the time of the first administration of the compound. In yet another embodiment, the absolute risk reduction rate (i.e., the statistically significant absolute amount by which the overall risk is reduced) for any one of the events is at least 4.0%, for a patient having a plasma Lp(a) concentration greater than or equal to 90 mg/dL prior to the time of the first administration of the compound. In another embodiment, the absolute risk reduction rate (i.e., the statistically significant absolute amount by which the overall risk is reduced) for any one of the events is at least 4.5%, for a patient having a plasma Lp(a) concentration greater than or equal to 90 mg/dL prior to the time of the first administration of the compound.

In other embodiments, the patient shows an improvement in any one of the following events or characteristics by a statistically significant amount at the end of the dosing period in comparison to patients who were not administered the compound, and wherein the patient has a plasma Lp(a) concentration ≥90 mg/dL prior to the time of the first administration of the compound: (i) the change in Lp(a) (in mg/dL and nmol/L) from baseline at specified time points selected from 1, 2, 3, 4, 5, 6, 9, 12, 13, 15, 18, 21, 24 and 27 months after treatment initiation, (ii) the change in expanded lipid profile parameters (total cholesterol, LDL-C, apoB, HDL-C, non-HDL-C, triglycerides) and hsCRP, (iii) the incidence of new onset type 2 diabetes mellitus, (iv) the quality of life as evaluated by the SF-12 questionnaire, and (v) the time to the first occurrence of the aortic valve replacement (open or trans-catheter) or hospitalization for aortic valve stenosis.

In one embodiment thereof, the patient having a plasma Lp(a) concentration ≥90 mg/dl prior to the time of the first administration of the compound shows an improvement in the following events or characteristics by a statistically significant amount at the end of the dosing period in comparison to patients who were not administered the compound: the change in Lp(a) (in mg/dL and nmol/L) from baseline at specified time points selected from 1, 2, 3, 4, 5, 6, 9, 12, 13, 15, 18, 21, 24 and 27 months after treatment initiation.

In one embodiment thereof, the patient having a plasma Lp(a) concentration ≥90 mg/dl prior to the time of the first administration of the compound shows an improvement in the following events or characteristics by a statistically significant amount at the end of the dosing period in comparison to patients who were not administered the compound: the change in expanded lipid profile parameters (total cholesterol, LDL-C, apoB, HDL-C, non-HDL-C, triglycerides) and hsCRP.

In one embodiment thereof, the patient having a plasma Lp(a) concentration ≥90 mg/dl prior to the time of the first administration of the compound shows an improvement in the following events or characteristics by a statistically significant amount at the end of the dosing period in comparison to patients who were not administered the compound: the incidence of new onset type 2 diabetes mellitus.

In one embodiment thereof, the patient having a plasma Lp(a) concentration ≥90 mg/dL prior to the time of the first administration of the compound shows an improvement in the following events or characteristics by a statistically significant amount at the end of the dosing period in comparison to patients who were not administered the compound: the quality of life as evaluated by the SF-12 questionnaire.

In one embodiment thereof, the patient having a plasma Lp(a) concentration ≥90 mg/dL prior to the time of the first administration of the compound shows an improvement in the following events or characteristics by a statistically significant amount at the end of the dosing period in comparison to patients who were not administered the compound: the time to the first occurrence of the aortic valve replacement (open or trans-catheter) or hospitalization for aortic valve stenosis.

In one embodiment thereof, the relative improvement rate (i.e., the statistically significant relative amount by which the event or characteristic is improved) is at least 15% for any one of the events or characteristics.

In one embodiment of any one of the above embodiments concerning the relative improvement rate (i.e., the statistically significant relative amount by which the event or characteristic is improved) is at least 15% for any one of the events for a patient having a plasma Lp(a) concentration greater than or equal to 90 mg/dL prior to the time of the first administration of the compound. In yet another embodiment, the relative improvement rate (i.e., the statistically significant relative amount by which the event or characteristic is improved) is at least 20% for a patient having a plasma Lp(a) concentration greater than or equal to 90 mg/dL prior to the time of the first administration of the compound. In another embodiment, the relative improvement rate (i.e., the statistically significant relative amount by which the event or characteristic is improved) is at least 25% for a patient having a plasma Lp(a) concentration greater than or equal to 90 mg/dL prior to the time of the first administration of the compound. In yet another embodiment, the relative improvement rate (i.e., the statistically significant relative amount by which the event or characteristic is improved) is at least 30% for a patient having a plasma Lp(a) concentration greater than or equal to 90 mg/dL prior to the time of the first administration of the compound. In another embodiment, the relative improvement rate (i.e., the statistically significant relative amount by which the event or characteristic is improved) is at least 35% for a patient having a plasma Lp(a) concentration greater than or equal to 90 mg/dL prior to the time of the first administration of the compound.

In some embodiments, the dosing period is at least six months. In another embodiment, the dosing period is at least one year. In yet another embodiment, the dosing period is at least two years. In another embodiment, the dosing period is at least three years.

In some embodiments, the patient receives a background therapy to achieve a guideline defined target low-density lipoprotein cholesterol (LDL-cholesterol) level.

In some embodiments, the background therapy comprises at least one of the following (i) a statin, (ii) ezetimibe, and (iii) a PCSK9 inhibitor.

In one embodiment, the background therapy comprises a statin and the patient receives an optimal dose of the statin before first administration of the compound.

In some embodiments, the patient has a sitting systolic blood pressure (SBP) less than 180 mmHg and/or diastolic BP (DBP) less than 110 mmHg.

In some embodiments, the patient has not been treated with niacin within a three month time period prior to the time of the first administration of the compound.

In some embodiments, the patient has not been diagnosed with heart failure New York Heart Association (NYHA) Class IV at the time of the first administration of the compound.

In some embodiments, the patient does not have a history of hemorrhagic stroke or other major bleeding prior to the time of the first administration of the compound.

In some embodiments, the patient has not had a myocardial infarction, stroke, coronary or lower limb re-vascularization, major cardiac or non-cardiac surgery, or lipoprotein apheresis within 3 months of the time of the first administration of the compound.

In some embodiments, the patient has no known active infection or major hematologic, renal, metabolic, gastrointestinal, or endocrine dysfunction.

In some embodiments, the patient has an estimated glomerular filtration rate (eGFR) greater than 30 mL/min/1.73 m² prior to the time of the first administration of the compound.

In some embodiments, the patient does not have an estimated glomerular filtration rate (eGFR) smaller than 30 mL/min/1.73 m² prior to the time of the first administration of the compound.

In some embodiments, the patient does not have active liver disease or hepatic dysfunction defined as aspartate aminotransferase (AST) or alanine aminotransferase (ALT) serum level more than 2 times the upper limit of normal (ULN) prior to the time of the first administration of the compound.

In some embodiments, the patient does not have a total bilirubin of more than 1.5 times the upper limit of normal (ULN) prior to the time of the first administration of the compound.

The present disclosure also provides a kit for treating, preventing, or ameliorating a disease, disorder or condition as described herein wherein the kit comprises: (i) ISIS 681257; and optionally (ii) a second agent or therapy as described herein.

A kit of the present invention can further include instructions for using the kit to treat, prevent, or ameliorate a disease, disorder or condition as described herein by combination therapy as described herein.

### Apo(a) Therapeutic Indications

The present disclosure provides methods for using ISIS 681257, which is a conjugated antisense compound targeted to an apo(a) nucleic acid for modulating the expression of apo(a) in a subject. When administered to a human, ISIS 681257 reduces expression of apo(a).

In certain embodiments, the invention provides methods for using ISIS 681257 in a pharmaceutical composition for treating a subject. In certain embodiments, the individual has an apo(a) related disease. In certain embodiments, the individual has an Lp(a) related disease. In certain embodiments, the individual has an inflammatory, cardiovascular and/or a metabolic disease, disorder or condition. In certain embodiments, the subject has an inflammatory, cardiovascular and/or metabolic disease, disorder or condition.

In certain embodiments, the cardiovascular diseases, disorders or conditions (CVD) include, but are not limited to, elevated Lp(a) associated CVD risk, recurrent cardiovascular events with elevated Lp(a), aortic stenosis (e.g., calcific aortic valve stenosis associated with high Lp(a)), aneurysm (e.g., abdominal aortic aneurysm), angina, arrhythmia, atherosclerosis, cerebrovascular disease, coronary artery disease, coronary heart disease (CHD), acute myocardial infarction (AMI), chronic CHD, arterial hypertension (HT), cerebrovascular stroke dyslipidemia, hypercholesterolemia, hyperlipidemia, hypertension, hypertriglyceridemia, myocardial infarction, peripheral vascular disease (e.g., peripheral artery disease), stroke and the like.

In certain embodiments, the cardiovascular events include, but are not limited to, major adverse cardiovascular event (MACE) (e.g, cardiovascular death, non-fatal myocardial infarction, non-fatal stroke, and urgent coronary re-vascularization requiring hospitalization, etc.), death from any cause, coronary heart disease (CHD) death, acute myocardial infarction (AMI) death, heart failure (HF) death, death caused by the immediate complications of a cardiac procedure, and urgent lower limb re-vascularization or amputation for ischemia and the like.

In certain embodiments, ISIS 681257 modulates physiological markers or phenotypes of the cardiovascular disease, disorder or condition. For example, administration of ISIS 681257 to a human can decrease Lp(a), LDL and cholesterol levels compared to untreated subjects. In certain embodiments, the modulation of the physiological markers or phenotypes can be associated with inhibition of apo(a) by ISIS 681257.

In certain embodiments, the physiological markers of the cardiovascular disease, disorder or condition can be quantifiable. For example, Lp(a), LDL or cholesterol levels can be measured and quantified by, for example, standard lipid tests. For such markers, in certain embodiments, the marker can be decreased by about 5, 10, 15, 20, 25, 30, 35, 40, 45, 50, 55, 60, 65, 70, 75, 80, 85, 90, 95 or 99%, or a range defined by any two of these values.

Also, provided herein are methods for preventing, treating or ameliorating a symptom associated with the cardiovascular disease, disorder or condition in a subject in need thereof. In certain embodiments, provided is a method for reducing the rate of onset of a symptom associated with the cardiovascular disease, disorder or condition. In certain embodiments, provided is a method for reducing the severity of a symptom associated with the cardiovascular disease, disorder or condition. In such embodiments, the methods comprise administering a therapeutically effective amount of ISIS 681257 to an individual in need thereof.

The cardiovascular disease, disorder or condition can be characterized by numerous physical symptoms. Any symptom known to one of skill in the art to be associated with the cardiovascular disease, disorder or condition can be prevented, treated, ameliorated or otherwise modulated with the compounds and methods described herein. In certain embodiments, the symptom can be any of, but not limited to, angina, chest pain, shortness of breath, palpitations, weakness, dizziness, nausea, sweating, tachycardia, bradycardia, arrhythmia, atrial fibrillation, swelling in the lower extremities, cyanosis, fatigue, fainting, numbness of the face, numbness of the limbs, claudication or cramping of muscles, bloating of the abdomen or fever.

In certain embodiments, the metabolic diseases, disorders or conditions include, but are not limited to, hyperglycemia, prediabetes, diabetes (type I and type II), obesity, insulin resistance, metabolic syndrome and diabetic dyslipidemia.

In certain embodiments, ISIS 681257 modulates physiological markers or phenotypes of the metabolic disease, disorder or condition. For example, administration of ISIS 681257 to humans can decrease glucose and insulin resistance levels in those subjects compared to untreated subjects. In certain embodiments, the modulation of the physiological markers or phenotypes can be associated with inhibition of apo(a) by ISIS 681257.

In certain embodiments, physiological markers of the metabolic disease, disorder or condition can be quantifiable. For example, glucose levels or insulin resistance can be measured and quantified by standard tests known in the art. For such markers, in certain embodiments, the marker can be decreased by about 5, 10, 15, 20, 25, 30, 35, 40, 45, 50, 55, 60, 65, 70, 75, 80, 85, 90, 95 or 99%, or a range defined by any two of these values. In another example, insulin sensitivity can be measured and quantified by standard tests known in the art. For such markers, in certain embodiments, the marker can be increase by about 5, 10, 15, 20, 25, 30, 35, 40, 45, 50, 55, 60, 65, 70, 75, 80, 85, 90, 95 or 99%, or a range defined by any two of these values.

Also, provided herein are methods for preventing, treating or ameliorating a symptom associated with the metabolic disease, disorder or condition in a subject in need thereof. In certain embodiments, provided is a method for reducing the rate of onset of a symptom associated with the metabolic disease, disorder or condition. In certain embodiments, provided is a method for reducing the severity of a symptom associated with the metabolic disease, disorder or condition. In such embodiments, the methods comprise administering a therapeutically effective amount of ISIS 681257 to an individual in need thereof.

The metabolic disease, disorder or condition can be characterized by numerous physical symptoms. Any symptom known to one of skill in the art to be associated with the metabolic disease, disorder or condition can be prevented, treated, ameliorated or otherwise modulated with the compounds and methods described herein. In certain embodiments, the symptom can be any of, but not limited to, excessive urine production (polyuria), excessive thirst and increased fluid intake (polydipsia), blurred vision, unexplained weight loss and lethargy.

In certain embodiments, the inflammatory diseases, disorders or conditions include, but are not limited to, elevated Lp(a) associated CVD risk, recurrent cardiovascular events with elevated Lp(a), aortic stenosis (e.g., calcific aortic valve stenosis associated with high Lp(a)), coronary artery disease (CAD), Alzheimer's Disease and thromboembolic diseases, disorder or conditions. Certain thromboembolic diseases, disorders or conditions include, but are not limited to, stroke, thrombosis, myocardial infarction and peripheral vascular disease.

In certain embodiments, ISIS 681257 modulates physiological markers or phenotypes of the inflammatory disease, disorder or condition. For example, administration of ISIS 681257 to a human can decrease inflammatory cytokine or other inflammatory markers levels in compared to untreated subjects. In certain embodiments, the modulation of the physiological markers or phenotypes can be associated with inhibition of apo(a) by ISIS 681257.

In certain embodiments, the physiological markers of the inflammatory disease, disorder or condition can be quantifiable. For example, cytokine levels can be measured and quantified by standard tests known in the art. For such markers, in certain embodiments, the marker can be decreased by at least about 5%, 10%, 15%, 20%, 25%, 30%, 35%, 40%, 45%, 50%, 55%, 60%, 65%, 70%, 75%, 80%, 85%, 90%, 95% or 99%, or a range defined by any two of these values.

Also, provided herein are methods for preventing, treating or ameliorating a symptom associated with the inflammatory disease, disorder or condition in a subject in need thereof. In certain embodiments, provided is a method for reducing the rate of onset of a symptom associated with the inflammatory disease, disorder or condition. In certain embodiments, provided is a method for reducing the severity of a symptom associated with the inflammatory disease, disorder or condition. In such embodiments, the methods comprise administering a therapeutically effective amount of ISIS 681257 to an individual in need thereof.

In certain embodiments, provided are methods of treating an individual with an apo(a) related disease, disorder or condition comprising administering a therapeutically effective amount of one or more pharmaceutical compositions as described herein. In certain embodiments, the individual has elevated apo(a) levels. In certain embodiments, provided are methods of treating an individual with an Lp(a) related disease, disorder or condition comprising administering a therapeutically effective amount of one or more pharmaceutical compositions as described herein. In certain embodiments, the individual has elevated Lp(a) levels. In certain embodiments, the individual has an inflammatory, cardiovascular and/or metabolic disease, disorder or condition. In certain embodiments, administration of a therapeutically effective amount of ISIS 681257 is accompanied by monitoring of apo(a) or Lp(a) levels. In certain embodiments, administration of a therapeutically effective amount of ISIS 681257 is accompanied by monitoring of markers of inflammatory, cardiovascular and/or metabolic disease, or other disease process associated with the expression of apo(a), to determine an individual's response to ISIS 681257. An individual's response to administration of ISIS 681257 can be used by a physician to determine the amount and duration of therapeutic intervention with the ISIS 681257.

In certain embodiments, administration of ISIS 681257 results in reduction of apo(a) expression by at least about 15%, 20%, 25%, 30%, 35%, 40%, 45%, 50%, 55%, 60%, 65%, 70%, 75%, 80%, 85%, 90%, 95%, or 99%, or a range defined by any two of these values. In certain embodiments, apo(a) expression is reduced to at least ≤100 mg/dL, ≤90 mg/dL, ≤80 mg/dL, ≤ 70 mg/dL, ≤60 mg/dL, ≤50 mg/dL, ≤40 mg/dL, ≤30 mg/dL, ≤20 mg/dL or ≤10 mg/dL.

In certain embodiments, administration of ISIS 681257 results in reduction of Lp(a) expression by at least about 15%, 20%, 25%, 30%, 35%, 40%, 45%, 50%, 55%, 60%, 65%, 70%, 75%, 80%, 85%, 90%, 95%, or 99%, or a range defined by any two of these values. In certain embodiments, Lp(a) expression is reduced to at least ≤200 mg/dL, ≤190 mg/dL, ≤180 mg/dL, ≤ 175 mg/dL, ≤170 mg/dL, ≤160 mg/dL, ≤150 mg/dL, ≤140 mg/dL, ≤130 mg/dL, ≤120 mg/dL, ≤110 mg/dL, ≤100 mg/dL, ≤90 mg/dL, ≤80 mg/dL, ≤70 mg/dL, ≤60 mg/dL, ≤55 mg/dL, ≤50 mg/dL, ≤45 mg/dL, ≤40 mg/dL, ≤35 mg/dL, ≤30 mg/dL, ≤25 mg/dL, ≤20 mg/dL, ≤15 mg/dL, or ≤10 mg/dL.

In certain embodiments, the invention provides methods for using ISIS 681257 in the preparation of a medicament. In certain embodiments, pharmaceutical compositions comprising ISIS 681257 are used for the preparation of a medicament for treating a patient suffering or susceptible to an inflammatory, cardiovascular and/or a metabolic disease, disorder or condition.

### Apo(a) Treatment Populations

Certain subjects with high Lp(a) levels are at a significant risk of various diseases (Lippi et al., Clinica Chimica Acta, 2011, 412:797-801; Solfrizz et al.). For example, subjects will Lp(a) levels greater than or equal to 75 nanomoles/liter (nmol/L) or ≥30 mg/dL are considered to have increased risk for various diseases. In many subjects with high Lp(a) levels, current treatments cannot reduce their Lp(a) levels to safe levels. Apo(a) plays an important role in the formation of Lp(a), hence reducing apo(a) can reduce Lp(a) and prevent, treat or ameliorate a disease associated with Lp(a).

In certain embodiments, treatment with the compounds and methods disclosed herein is indicated for a human with elevated apo(a) levels and/or Lp(a) levels. In certain embodiments, the human has apo(a) levels ≥10 mg/dl, ≥20 mg/dL, ≥30 mg/dL, ≥40 mg/dL, ≥50 mg/dl, ≥60 mg/dL, ≥70 mg/dL, ≥80 mg/dL, ≥90 mg/dL, or ≥100 mg/dL.

In certain embodiments, the human has Lp(a) levels ≥70 mg/dL, ≥80 mg/dL, ≥90 mg/dL, ≥100 mg/dL, ≥110 mg/dL, ≥120 mg/dL, ≥130 mg/dL, ≥140 mg/dL, ≥150 mg/dl, ≥160 mg/dL, ≥170 mg/dL, ≥175 mg/dL, ≥180 mg/dL, ≥190 mg/dL, or ≥200 mg/dL.

In certain embodiments, the human has apo(a) levels greater than the upper limit of normal, e.g, wherein the human has apo(a) levels ≥30 mg/dL, ≥35 mg/dL, ≥40 mg/dl, ≥50 mg/dl, ≥60 mg/dL, ≥70 mg/dL, ≥80 mg/dL, ≥90 mg/dL, ≥100 mg/dL, ≥110 mg/dl, ≥120 mg/dL, ≥130 mg/dL, ≥140 mg/dL, ≥150 mg/dL, ≥160 mg/dL, ≥170 mg/dL, ≥175 mg/dL, ≥180 mg/dL, ≥190 mg/dL, or ≥200 mg/dL.

In certain embodiments, the human patient has an Lp(a) level ≥70 mg/dL prior to the time of the first administration of the compound (i.e. before the treatment start).

In certain embodiments, the human patient receives an LDL-cholesterol lowering treatment as follows:
an optimal LDL-C lowering treatment to meet the target LDL-C level according to local practice/guidelines, or
the highest tolerated doses of statins and/or with other optimized LDL-lowering therapy (e.g. ezetimibe, cholesterol absorption inhibitor, fibrate, PCSK9 inhibitor), or
another optimized LDL-lowering therapy (e.g. ezetimibe, cholesterol absorption inhibitor, fibrate, PCSK9 inhibitor) according to local practice/guidelines.

In certain embodiments, the human patient has an established cardiovascular disease. The established CV disease defined as ANY of the following three conditions:
1. History of spontaneous myocardial infarction (not resulting from PCI or CABG) having occurred in the period ≥3 months to ≤10 years prior to the screening visit and documented as follows:
   (i) Acute MI (hospitalization records): requires documentation of a rise and/or fall of cardiac biomarkers (preferably cardiac troponin) with at least one value above the 99th percentile of the upper reference limit (URL) and at least one of the following:
      a. Symptoms of ischemia
      b. ECG changes indicative of acute myocardial ischemia (new ST-T changes or new LBBB; please see Appendix 2 for ST and T-wave changes)
      c. Development of pathological Q waves (please see Appendix 2 for definitions of pathological Q waves)
      d. Imaging evidence of new loss of viable myocardium or new regional wall motion abnormality in a pattern consistent with an ischemic etiology
      e. Identification of a coronary thrombus by angiography including intracoronary imaging
   (ii) Prior MI (no complete hospital records for acute event available): requires documentation of any one of the following:
      a. Pathological Q waves, with or without symptoms, in the absence of a non-ischemic cause,
      b. Imaging evidence of a region of loss of viable myocardium that is thinned and fails to contract, in the absence of a non-ischemic cause
2. History of ischemic stroke (an acute episode of focal cerebral, spinal, or retinal dysfunction caused by infarction of central nervous system tissue) having occurred in the period ≥3 months to ≤10 years prior to the screening visit documented by CT scan, MRI or other visualization method. Transient ischemic attack or embolic stroke (not of atherosclerotic origin) are not qualifying events.
3. Clinically significant symptomatic peripheral artery disease, evidenced by intermittent claudication with an ankle-brachial index ≤0.90 and/or limb amputation or re-vascularization due to lower limb ischemia. Thromboangiitis obliterans is not a qualifying event.

### Certain Apo(a) Dosing Regimens

In certain embodiments, between about 75 to about 85 mg of ISIS 681257 is administered to a human subject in need thereof as defined herein once monthly.

In certain embodiments, between about 75 to about 85 mg of ISIS 681257 is administered to a human subject in need thereof as defined herein once every 4 weeks.

In certain embodiments, about 80 mg of ISIS 681257 is administered to a human subject in need thereof as defined herein once monthly.

In certain embodiments, between about 80 mg of ISIS 681257 is administered to a human subject in need thereof as defined herein once every 4 weeks.

In certain embodiments, the above amounts of ISIS 681257 are administered to a human subject in need thereof as defined herein during a dosing period.

In certain embodiments, the dosing period is at least six months. In another embodiment, the dosing period is at least one year. In yet another embodiment, the dosing period is at least two years. In another embodiment, the dosing period is three years. In another embodiment, the dosing period is at least four years.

In certain embodiments, 80 mg of ISIS 681257 is administered to a human subject in need thereof as defined every four weeks during a dosing period. In another embodiment, 80 mg of ISIS 681257 is administered to a human subject in need thereof as defined every four weeks for at least six months. In another embodiment, 80 mg of ISIS 681257 is administered to a human subject in need thereof as defined every four weeks for at least one year. In another embodiment, 80 mg of ISIS 681257 is administered to a human subject in need thereof as defined every four weeks for at least two years. In another embodiment, 80 mg of ISIS 681257 is administered to a human subject in need thereof as defined every four weeks for three years. In another embodiment, 80 mg of ISIS 681257 is administered to a human subject in need thereof as defined every four weeks for at least four years.

In certain embodiments, 80 mg of ISIS 681257 is administered to a human subject in need thereof as defined once a month during a dosing period. In another embodiment, 80 mg of ISIS 681257 is administered to a human subject in need thereof as defined once a month for at least six months. In another embodiment, 80 mg of ISIS 681257 is administered to a human subject in need thereof as defined once a month for at least one year. In another embodiment, 80 mg of ISIS 681257 is administered to a human subject in need thereof as defined once a month for at least two years. In another embodiment, 80 mg of ISIS 681257 is administered to a human subject in need thereof as defined once a month for three years. In another embodiment, 80 mg of ISIS 681257 is administered to a human subject in need thereof as defined once a month for at least four years.

### Certain Treatment and Dosing Regimens

In certain embodiments, the present disclosure relates to a method of reducing the risk of a cardiovascular event in a patient who has established cardiovascular disease comprising, administering a unit dose comprising from about 75 mg to about 85 mg of the compound ISIS 681257 by subcutaneous injection to the patient once a month, wherein said patient has a plasma Lp(a) concentration greater than or equal to 70 mg/dL prior to the time of the first administration of the compound, and wherein the cardiovascular event is selected from a major adverse cardiovascular event (MACE), all cause death, coronary heart disease (CHD) death, acute myocardial infarction (AMI) death, heart failure (HF) death, death caused by the immediate complications of a cardiac procedure, and urgent lower limb re-vascularization or amputation for ischemia.

In another embodiment, the present disclosure relates to a method of reducing the risk of a cardiovascular event in a patient who has established cardiovascular disease comprising, administering a unit dose comprising from about 75 mg to about 85 mg of the compound ISIS 681257 by subcutaneous injection to the patient once a month, wherein said patient has a plasma Lp(a) concentration greater than or equal to 70 mg/dL prior to the time of the first administration of the compound, and wherein the patient who has established cardiovascular disease is a patient having at least one of the following (i) a history of spontaneous myocardial infarction, (i) a history of ischemic stroke, and (iii) clinically significant symptomatic peripheral artery disease.

In another embodiment, the present disclosure relates to a method of reducing the risk of a cardiovascular event in a patient who has established cardiovascular disease comprising, administering a unit dose comprising from about 75 mg to about 85 mg of the compound ISIS 681257 by subcutaneous injection to the patient once a month, wherein said patient has a plasma Lp(a) concentration greater than or equal to 70 mg/dL prior to the time of the first administration of the compound, and wherein the cardiovascular event is selected from a major adverse cardiovascular event (MACE), all cause death, coronary heart disease (CHD) death, acute myocardial infarction (AMI) death, heart failure (HF) death, death caused by the immediate complications of a cardiac procedure, and urgent lower limb re-vascularization or amputation for ischemia; and wherein the patient who has established cardiovascular disease is a patient having at least one of the following (i) a history of spontaneous myocardial infarction, (i) a history of ischemic stroke, and (iii) clinically significant symptomatic peripheral artery disease.

In another embodiment, the present disclosure relates to a method of reducing the risk of a cardiovascular event in a patient who has established cardiovascular disease comprising, administering a unit dose comprising from about 77 mg to about 82 mg of the compound ISIS 681257 by subcutaneous injection to the patient once a month, wherein said patient has a plasma Lp(a) concentration greater than or equal to 70 mg/dL prior to the time of the first administration of the compound, wherein the cardiovascular event is selected from a major adverse cardiovascular event (MACE), all cause death, coronary heart disease (CHD) death, acute myocardial infarction (AMI) death, heart failure (HF) death, death caused by the immediate complications of a cardiac procedure, and urgent lower limb re-vascularization or amputation for ischemia; and wherein the patient who has established cardiovascular disease is a patient having at least one of the following (i) a history of spontaneous myocardial infarction, (i) a history of ischemic stroke, and (iii) clinically significant symptomatic peripheral artery disease.

In another embodiment, the present disclosure relates to a method of reducing the risk of a cardiovascular event in a patient who has established cardiovascular disease, comprising, administering a unit dose comprising about 80 mg of the compound ISIS 681257 by subcutaneous injection to the patient once a month, wherein said patient has a plasma Lp(a) concentration greater than or equal to 70 mg/dL prior to the time of the first administration of the compound, wherein the cardiovascular event is selected from a major adverse cardiovascular event (MACE), all cause death, coronary heart disease (CHD) death, acute myocardial infarction (AMI) death, heart failure (HF) death, death caused by the immediate complications of a cardiac procedure, and urgent lower limb re-vascularization or amputation for ischemia; and wherein the patient who has established cardiovascular disease is a patient having at least one of the following (i) a history of spontaneous myocardial infarction, (i) a history of ischemic stroke, and (iii) clinically significant symptomatic peripheral artery disease.

In another embodiment, the present disclosure relates to a method of reducing the risk of a cardiovascular event in a patient who has established cardiovascular disease, comprising, administering a unit dose comprising about 80 mg of the compound ISIS 681257 by subcutaneous injection to the patient once a month, wherein said patient has a plasma Lp(a) concentration greater than or equal to 70 mg/dL prior to the time of the first administration of the compound, wherein the cardiovascular event is selected from a major adverse cardiovascular event (MACE), all cause death, coronary heart disease (CHD) death, acute myocardial infarction (AMI) death, heart failure (HF) death, death caused by the immediate complications of a cardiac procedure, and urgent lower limb re-vascularization or amputation for ischemia; wherein the patient who has established cardiovascular disease is a patient having at least one of the following (i) a history of spontaneous myocardial infarction, (i) a history of ischemic stroke, and (iii) clinically significant symptomatic peripheral artery disease; and wherein the relative risk reduction rate for any one of the events is (i) at least 15%, preferably at least 20%, more preferably at least 25%, for a patient having a plasma Lp(a) concentration greater than or equal to 70 mg/dL prior to the time of the first administration of the compound; (ii) at least 20%, preferably at least 25%, more preferably at least 30%, for a patient having a plasma Lp(a) concentration greater than or equal to 90 mg/dL prior to the time of the first administration of the compound.

In another embodiment, the present disclosure relates to a method of reducing the risk of a cardiovascular event in a patient who has established cardiovascular disease, comprising, administering a unit dose comprising no more than 80 mg of the compound ISIS 681257 by subcutaneous injection to the patient once a month, wherein said patient has a plasma Lp(a) concentration greater than or equal to 70 mg/dL prior to the time of the first administration of the compound, wherein the cardiovascular event is selected from a major adverse cardiovascular event (MACE), all cause death, coronary heart disease (CHD) death, acute myocardial infarction (AMI) death, heart failure (HF) death, death caused by the immediate complications of a cardiac procedure, and urgent lower limb re-vascularization or amputation for ischemia; and wherein the patient who has established cardiovascular disease is a patient having at least one of the following (i) a history of spontaneous myocardial infarction, (i) a history of ischemic stroke, and (iii) clinically significant symptomatic peripheral artery disease.

In certain embodiments, the patient receives a background therapy to achieve a guideline defined target low-density lipoprotein cholesterol (LDL-cholesterol) level; the patient has a sitting systolic blood pressure (SBP) less than 180 mmHg and/or diastolic BP (DBP) less than 110 mmHg, the patient has not been treated with niacin within a three month time period prior to the time of the first administration of the compound; the patient has not been diagnosed with heart failure of Heart failure New York Heart Association (NYHA) Class IV at the time of the first administration of the compound; the patient does not have a history of hemorrhagic stroke or other major bleeding prior to the time of the first administration of the compound; the patient has not had a myocardial infarction, stroke, coronary or lower limb re-vascularization, major cardiac or non-cardiac surgery, or lipoprotein apheresis within 3 months of the time of the first administration of the compound; the patient has no known active infection or major hematologic, renal, metabolic, gastrointestinal, or endocrine dysfunction; the patient has an estimated glomerular filtration rate (eGFR) greater than 30 mL/min/1.73 m² prior to the time of the first administration of the compound; the patient does not have an estimated glomerular filtration rate (eGFR) smaller than 30 mL/min/1.73 m² prior to the time of the first administration of the compound; the patient does not have active liver disease or hepatic dysfunction defined as aspartate aminotransferase (AST) or alanine aminotransferase (ALT) serum level more than 2 times the upper limit of normal (ULN) prior to the time of the first administration of the compound; or the patient does not have a total bilirubin of more than 1.5 times the upper limit of normal (ULN) prior to the time of the first administration of the compound or a combination thereof.

### Certain Pharmaceutical Compositions

In certain embodiments, the present disclosure provides pharmaceutical compositions comprising one or more antisense compound. In certain embodiments, such pharmaceutical composition comprises a suitable pharmaceutically acceptable diluent or carrier. In certain embodiments, a pharmaceutical composition comprises a sterile saline solution and one or more antisense compound. In certain embodiments, such pharmaceutical composition consists of a sterile saline solution and one or more antisense compound. In certain embodiments, the sterile saline is pharmaceutical grade saline. In certain embodiments, a pharmaceutical composition comprises one or more antisense compound and sterile water. In certain embodiments, a pharmaceutical composition consists of one or more antisense compound and sterile water. In certain embodiments, the sterile saline is pharmaceutical grade water. In certain embodiments, a pharmaceutical composition comprises one or more antisense compound and phosphate-buffered saline (PBS). In certain embodiments, a pharmaceutical composition consists of one or more antisense compound and sterile phosphate-buffered saline (PBS). In certain embodiments, the sterile saline is pharmaceutical grade PBS.

In certain embodiments, the sterile liquid is water. In another embodiment, the sterile liquid is water with a sodium phosphate buffer. In another embodiment, the sterile liquid is water with a sodium phosphate buffer and sodium chloride.

In certain embodiments, the compound is formulated in not more than 1.3 mL of the sterile liquid. In another embodiment, the compound is formulated in not more than 1.2 mL of the sterile liquid. In another embodiment, the compound is formulated in not more than 1.0 mL of the sterile liquid. In another embodiment, the compound is formulated in not more than 0.8 mL of the sterile liquid. In another embodiment, the compound is formulated in not more than 0.5 mL of the sterile liquid. In yet another embodiment, the compound is formulated in not more than 0.4 mL of the sterile liquid. In another embodiment, the compound is formulated in not more than 0.25 mL of the sterile liquid. In yet another embodiment, the compound is formulated in not more than 0.2 mL of the sterile liquid. In another embodiment, the compound is formulated in not more than 0.1 mL of the sterile liquid. In yet another embodiment, the compound is formulated in not more than 0.05 mL of the sterile liquid.

In certain embodiments, the compound is formulated in about 1.3 mL of the sterile liquid. In another embodiment, the compound is formulated in about 1.2 mL of the sterile liquid. In another embodiment, the compound is formulated in about 1.2 mL of the sterile liquid. In another embodiment, the compound is formulated in about 1.0 mL of the sterile liquid. In another embodiment, the compound is formulated in about 0.8 mL of the sterile liquid. In another embodiment, the compound is formulated in about 0.5 mL of the sterile liquid. In yet another embodiment, the compound is formulated in about 0.4 mL of the sterile liquid. In another embodiment, the compound is formulated in about 0.25 mL of the sterile liquid. In yet another embodiment, the compound is formulated in about 0.2 mL of the sterile liquid.

In certain embodiments, antisense compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions provided herein comprise one or more modified oligonucleotides and one or more excipients. In certain such embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, a pharmaceutical composition provided herein comprises a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, a pharmaceutical composition is prepared for administration by injection (e.g., intravenous, subcutaneous, intramuscular, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the pharmaceutical agents to allow for the preparation of highly concentrated solutions.

In certain embodiments, the present disclosure provides methods of administering a pharmaceutical composition comprising an oligonucleotide of the present disclosure to a subject. Suitable administration routes include parenteral (e.g., intravenous, intramuscular, intramedullary, and subcutaneous).

### Compounds for Use

All the aforementioned embodiments for the methods of treatment according to the present invention are equally applicable to
the use of the compound ISIS 681257 as defined herein or a pharmaceutical composition comprising the compound ISIS 681257 as defined herein for the manufacture of a medicament for use according to the present invention,
the use of the compound ISIS 681257 as defined herein or a pharmaceutical composition comprising the compound ISIS 681257 as defined herein according to the present invention,
the compound ISIS 681257 as defined herein or a pharmaceutical composition comprising the compound ISIS 681257 as defined herein for use according to the present invention.

In particular, all the aforementioned embodiments for the methods of reducing the risk of a cardiovascular event in a patient who has established cardiovascular disease comprising, administering to said patient a unit dose comprising from about 75 mg to about 85 mg of the compound ISIS 681257 by subcutaneous injection once a month or once every four weeks, wherein said patient has a plasma Lp(a) concentration greater than or equal to 70 mg/dL prior to the time of the first administration of the compound, are equally applicable to the compound ISIS 681257 as defined herein or a pharmaceutical composition comprising the compound ISIS 681257 as defined herein for the use in reducing the risk of a cardiovascular event in a patient who has established cardiovascular disease comprising administering to said patient a unit dose comprising from about 75 mg to about 85 mg of the compound ISIS 681257 by subcutaneous injection once a month or once every four weeks, wherein said patient has a plasma Lp(a) concentration greater than or equal to 70 mg/dL prior to the time of the first administration of the compound.
the use of the compound ISIS 681257 as defined herein or a pharmaceutical composition comprising the compound ISIS 681257 as defined herein in the manufacture of a medicament in the form of a unit dose for subcutaneous injection comprising from about 75 mg to about 85 mg of the compound ISIS 681257, wherein the medicament is to be administered once a month or once every four weeks to a patient who has established cardiovascular disease for reducing the risk of a cardiovascular event, wherein said patient has a plasma Lp(a) concentration greater than or equal to 70 mg/dL prior to the time of the first administration of the compound.
the compound ISIS 681257 as defined herein or a pharmaceutical composition comprising the compound ISIS 681257 as defined herein for use as a medicament in the form of a unit dose for subcutaneous injection comprising from about 75 mg to about 85 mg of the compound ISIS 681257, wherein the medicament is to be administered once a month or once every four weeks to a patient who has established cardiovascular disease for reducing the risk of a cardiovascular event, wherein said patient has a plasma Lp(a) concentration greater than or equal to 70 mg/dL prior to the time of the first administration of the compound.
use of the compound ISIS 681257 as defined herein or a pharmaceutical composition comprising the compound ISIS 681257 as defined herein as a medicament in the form of a unit dose for subcutaneous injection comprising from about 75 mg to about 85 mg of the compound ISIS 681257, wherein the medicament is to be administered once a month or once every four weeks to a patient who has established cardiovascular disease for reducing the risk of a cardiovascular event, wherein said patient has a plasma Lp(a) concentration greater than or equal to 70 mg/dL prior to the time of the first administration of the compound

### Non-Limiting Disclosure and Incorporation by Reference

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH for the natural 2′-H of DNA) or as an RNA having a modified base (thymine (methylated uracil) for natural uracil of RNA).

Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligonucleotide having the nucleobase sequence “ATCGATCG” encompasses any oligonucleotides having such nucleobase sequence, whether modified or unmodified, including, but not limited to, such compounds comprising RNA bases, such as those having sequence “AUCGAUCG” and those having some DNA bases and some RNA bases such as “AUCGATCG” and oligonucleotides having other modified bases, such as “AT^{me}CGAUCG,” wherein m^{eC} indicates a cytosine base comprising a methyl group at the 5-position.

### EXAMPLES

The disclosure is further illustrated by the following examples which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1: a Randomized Double-Blind, Placebo-Controlled, Multicenter Study Assessing the Impact of Lipoprotein (a) Lowering with ISIS 681257 on Major Cardiovascular Events in Patients with Established Cardiovascular Disease

### LIST OF ABBREVIATIONS

| | |
|---|---|
| ACR | albumin-creatinine ratio |
| ADA | anti-drug antibodies |
| AE | adverse event |
| AESI | adverse event of special interest |
| ALP | alkaline phosphatase |
| ALT | alanine aminotransferase |
| apo(a) | apolipoprotein(a) |
| apoB | apolipoprotein B |
| ASO | antisense oligonucleotides |
| AST | aspartate aminotransferase |
| AUC | Area under the concentration-time curve |
| AV | aortic valve |
| BMI | Body Mass Index |
| BUN | blood urea nitrogen |
| CEC | clinical endpoint committee |
| CFR | Code of Federal Regulation |
| CHD | coronary heart disease |
| CRO | Contract Research Organization |
| CTT | Clinical trial team |
| CV | cardiovascular |
| CVD | cardiovascular disease |
| DAR | dose administration record |
| DBP | diastolic blood pressure |
| DILI | drug-induced liver injury |
| DLT | dose-limiting toxicity |
| DMC | Data Monitoring Committee |
| EC | executive committee |
| ECG | Electrocardiogram |
| eCRF | electronic Case Report Form |
| EDC | Electronic Data Capture |
| eGFR | Estimated glomerular filtration rate |
| eSAE | Electronic Serious Adverse Event |
| FAS | full analysis set |
| FDA | Food and Drug Administration |
| GCP | Good Clinical Practice |
| GCS | Global Clinical Supply |
| GGT | gamma-glutamly transferase |
| HIV | human immunodeficiency virus |
| IA | Interim Analysis |
| IB | Investigator's Brochure |
| ICF | Informed consent form |
| ICH | International Conference on Harmonization of |
| | Technical Requirements for Registration of |
| | Pharmaceuticals for Human Use |
| IEC | Independent Ethics Committee |
| IFU | instructions for use |
| IN | Investigator Notification |
| IRB | Institutional Review Board |
| IRT | Interactive Response Technology |
| IUD | intrauterine device |
| IUS | intrauterine system |
| LDH | lactate dehydrogenase |
| LDL | Low-density lipoprotein |
| LDL-C | low-density lipoprotein Cholesterol |
| LFT | Liver function test |
| LLN | lower limit of normal |
| LLOQ | lower limit of quantification |
| Lp(a) | Lipoprotein(a) |
| MACE | major adverse cardiovascular event |
| MALE | major adverse limb event |
| MedDRA | Medical dictionary for regulatory activities |
| mg | milligram(s) |
| MI | myocardial infarction |
| mL | milliliter(s) |
| ml | milliliter(s) |
| NOAEL | no observed adverse effect level |
| NSD | needle safety device |
| NYHA | New York Heart Association |
| oxPl | oxydized phospholipids |
| PAD | peripheral artery disease |
| PCR | Protein-creatinine ratio |
| PD | pharmacodynamic(s) |
| PK | pharmacokinetic(s) |
| Q2W | every second week |
| Q4W | every 4 weeks |
| QM | once per month |
| QW | once per week |
| RAS | randomized set |
| RBC | red blood cell(s) |
| RDC | Remote Data Capture |
| REB | Research Ethics Board |
| s.c. | subcutaneous |
| SAE | serious adverse event |
| SAF | safety set |
| SBP | systolic blood pressure |
| siRNA | small interfering ribonucleic acid |
| SUSAR | Suspected Unexpected Serious Adverse Reactions |
| TBIL | Total bilirubin |
| TBL | total bilirubin |
| ULN | upper limit of normal |
| URL | upper reference limit |
| WBC | white blood cell(s) |
| WHO | World Health Organization |

### Glossary of Terms

| | |
|---|---|
| Assessment | A procedure used to generate data required by the study |
| Cohort | A specific group of subjects fulfilling certain criteria |
| Control drug | A study drug used as a comparator to reduce assessment bias, preserve |
| | blinding of investigational drug, assess internal study validity, and/or evaluate |
| | comparative effects of the investigational drug. |
| Dosage | Dose of the study treatment given to the subject in a time unit (e.g. 100 mg once |
| | a day, 75 mg twice a day) |
| Enrollment | Point/time of subject entry into the study; the point at which informed consent |
| | must be obtained (i.e. prior to starting any of the procedures described in the |
| | protocol). |
| Epoch | Interval of time in the planned conduct of a study. An epoch is associated with a |
| | purpose (e.g. screening, randomization, treatment, follow-up), which applies |
| | across all arms of a study. |
| Investigational drug | The study drug whose properties are being tested in the study; this definition is |
| | consistent with US CFR 21 Section 312.3 and is synonymous with |
| | “investigational new drug” or “investigational medicinal product”. |
| Investigational | All investigational drug(s) whose properties are being tested in the study as well |
| treatment | as their associated treatment controls. This includes any placebos, any active |
| | controls, as well as approved drugs used outside of their indication/approved |
| | dosage or tested in a fixed combination. Investigational treatment generally |
| | does not include other treatments administered as concomitant background |
| | therapy required or allowed by the protocol when used within approved |
| | indication/dosage. |
| Medication number | A unique identifier on the label of each study drug package in studies that |
| | dispense study drug using an IRT system. |
| Medication pack | A unique identifier on the label of each drug package in studies that dispense |
| number | study treatment using an IRT system |
| Non-investigational | Products which are not the object of investigation (e.g. any background therapy |
| medicinal Product | administered to each of the clinical trial subjects, regardless of randomization |
| (NIMP) | group, rescue medication, active drug run-ins etc.) |
| Part | A single component of a study which contains different objectives or populations |
| | within that single study. Common parts within a study are: a single dose part |
| | and a multiple dose part, or a part in patients with established disease and in |
| | those with newly-diagnosed disease. |
| Patient | An individual with the condition of interest |
| Period | A minor subdivision of the study timeline; divides phases into smaller functional |
| | segments such as screening, baseline, titration, washout, etc. |
| Personal data | Subject information collected by the Investigator that is transferred to Novartis |
| | for the purpose of the clinical trial. This data includes subject identifier |
| | information, study information and biological samples |
| Premature subject | Point/time when the subject exits from the study prior to the planned completion |
| withdrawal | of all study drug administration and assessments; at this time all study drug |
| | administration is discontinued and no further assessments are planned. |
| Randomization | A unique identifier assigned to each randomized subject, corresponding to a |
| number | specific treatment arm assignment |
| Screen Failure | A subject who is screened but is not treated or randomized |
| Study completion | Point/time at which the subject came in for a final evaluation visit or when study |
| | drug was discontinued whichever is later. |
| Study drug | Point/time when subject permanently stops taking study drug for any reason; |
| discontinuation | may or may not also be the point/time of premature subject withdrawal. |
| Study | Any drug (or combination of drugs) administered to the subject as part of the |
| drug/treatment | required study procedures; includes investigational drug, active drug run-ins or |
| | background therapy. |
| Study treatment | Any drug administered to the study participants as part of the required study |
| | procedures; includes investigational drug (s), control(s) or non-investigational |
| | medicinal product(s) |
| Study treatment | When the subject permanently stops taking study treatment prior to the defined |
| discontinuation | study treatment completion date |
| Subject | An individual who has consented to participate in this study. The term Subject |
| | may be used to describe either a healthy volunteer or a patient. |
| Subject number | A unique number assigned to each subject upon signing the informed consent. |
| | This number is the definitive, unique identifier for the subject and should be |
| | used to identify the subject throughout the study for all data collected, sample |
| | labels, etc. |
| Treatment number | A unique identifier assigned in non-randomized studies to each dosed subject, |
| | corresponding to a specific treatment arm |
| Variable | Information used in the data analysis; derived directly or indirectly from data |
| | collected using specified assessments at specified timepoints. |
| Withdrawal of | Withdrawal of consent from the study is defined as when a subject does not |
| consent (WoC) | want to participate in the study any longer, and does not want any further visits |
| | or assessments, and does not want any further study related contact, and does |
| | not allow analysis of already obtained biologic material |

### Purpose

The study is a pivotal phase 3 study designed to test the hypothesis that treatment with ISIS 681257 80 mg subcutaneous (SC) once monthly (QM) will significantly reduce the risk of MACE, i.e. CV deaths, non-fatal myocardial infarction (MI), non-fatal stroke and urgent coronary re-vascularization in patients with established CVD and elevated levels of Lp(a) who are treated for CV risk factors other than Lp(a) according to local guidelines for the reduction of cardiovascular risk in patients with established CVD and elevated Lp(a).

### Objectives and Endpoints

Summary: The primary objectives of this study is to demonstrate the superiority of TQJ230 compared to placebo in reducing the risk of expanded MACE (cardiovascular death, non-fatal MI, non-fatal stroke and urgent coronary re-vascularization requiring hospitalization) in 1) the overall study population with established CVD (Lp(a)≥70 mg/dL) and/or 2) in a subpopulation with established CVD and Lp(a)≥90 mg/dL. The secondary objectives in the overall trial population and in the subpopulation (≥90 mg/dL) are (i) to demonstrate the superiority of TQJ230 compared to placebo in reducing the risk of the MACE composite of CV death, non-fatal MI and non-fatal stroke, and (ii) to demonstrate the superiority of TQJ230 compared to placebo in reducing the risk of the composite of coronary heart disease (CHD) outcomes: death due to CHD, non-fatal MI and urgent coronary re-vascularization requiring hospitalization, and (iii) to evaluate the rate of all cause death

The study has two primary objectives addressing the same scientific hypothesis: one in the full study population who is at a high risk of a CV event, and the other one in a subpopulation expected to be at higher risk, i.e patients with Lp(a) value ≥90 mg/dL. Successful achievement of the primary objectives requires meeting one of the two, or both primary objectives. An independent Clinical Endpoint Committee (CEC) will adjudicate all primary and secondary endpoints. Definitions of all endpoints will be included in the CEC Charter and Endpoints Manual, which will be provided to CEC and investigators, respectively.

### Primary Objective(s) and Endpoint(s):

To demonstrate the superiority of ISIS 681257 compared to placebo in reducing the risk of expanded MACE (cardiovascular death, non-fatal MI, non-fatal stroke and urgent coronary re-vascularization requiring hospitalization) in the overall study population with established CVD and (Lp(a)≥70 mg/dL). Endpoint: To assess the time to the first occurrence of CEC confirmed expanded MACE (cardiovascular death, non-fatal MI, non-fatal stroke and urgent coronary re-vascularization requiring hospitalization) in a population of patients with elevated Lp(a)≥70 mg/dL; and/or

To demonstrate the superiority of ISIS 681257 compared to placebo in reducing the risk of expanded MACE (cardiovascular death, non-fatal MI, non-fatal stroke and urgent coronary re-vascularization requiring hospitalization) in a subpopulation with established CVD and Lp(a)≥90 mg/dL. Endpoint: To assess the time to the first occurrence of CEC confirmed expanded MACE (cardiovascular death, non-fatal MI, non-fatal stroke and urgent coronary re-vascularization requiring hospitalization) in a population of patients with elevated Lp(a)≥90 mg/dL.

### Secondary Objective(s) and Endpoint(s):

In the overall trial population and in the sub-population (≥90 mg/dL):

Demonstrate the superiority of ISIS 681257 compared to placebo in reducing the risk of the MACE composite of CV death, non-fatal MI and non-fatal stroke. Endpoint: Time to the first occurrence of the CEC confirmed composite endpoint of MACE (CV death, non-fatal MI, and non-fatal stroke).

Demonstrate the superiority of ISIS 681257 compared to placebo in reducing the risk of the composite of coronary heart disease (CHD) outcomes: death due to CHD, non-fatal MI and urgent coronary re-vascularization requiring hospitalization. Endpoint: Time to the first occurrence of the CEC confirmed composite endpoint of CHD: CHD death, non-fatal MI, urgent coronary re-vascularization requiring hospitalization.

To evaluate the rate of all-cause death.

Endpoint: CEC confirmed all-cause death from randomization to the end of study.

### Exploratory Objective(s) and Endpoint(s):

In the overall trial population and in the sub-population (≥90 mg/dL):

Evaluate ISIS 681257 compared to placebo in reducing the risk of the composite of all-cause mortality, non-fatal MI and non-fatal stroke. Endpoint: Time to the first occurrence of the composite of all-cause death, non-fatal MI and non-fatal stroke.

Evaluate ISIS 681257 compared to placebo for the composite of total vascular events: CV death, non-fatal MI, non-fatal stroke, urgent coronary re-vascularization requiring hospitalization and urgent lower limb re-vascularization for or amputation due to ischemia. Endpoint: Time to the first occurrence of the composite endpoint of total vascular events: CV death, non-fatal MI, non-fatal stroke, urgent coronary re-vascularization requiring hospitalization and urgent lower limb re-vascularization for or amputation due to ischemia.

Evaluate ISIS 681257 compared to placebo for the composite endpoint of all-cause mortality, non-fatal MI, non-fatal stroke and urgent coronary re-vascularization requiring hospitalization. Endpoint: Time to the first occurrence of the composite endpoint of all-cause death, non-fatal MI, non-fatal stroke and urgent coronary re-vascularization requiring hospitalization.

Evaluate ISIS 681257 compared to placebo in reducing the risk of the composite of fatal and non-fatal stroke. Endpoint: Time to the first occurrence of CEC confirmed composite of fatal and non-fatal stroke.

Evaluate the rate of major adverse limb events (MALE) in patients with history of peripheral artery disease (PAD). Endpoint: Time to the first occurrence of acute lower limb ischemia, lower limb amputation due to or urgent lower limb re-vascularization for ischemia.

Evaluate the rate of hospitalization for unstable angina. Endpoint: Time to the first occurrence of hospitalization for unstable angina.

Evaluate the rate of hospitalizations for heart failure. Endpoint: Time to the first occurrence of hospitalization for heart failure.

Evaluate the change in Lp(a). Endpoint: Change in Lp(a) from baseline at selected time points.

Evaluate the change in expanded lipid profile parameters and hsCRP. Endpoint: Change in total cholesterol, LDL-C, apoB, HDL-C, non-HDL-C, triglycerides, and hsCRP from baseline at specified time points.

To evaluate the incidence of new onset type 2 diabetes mellitus. Endpoint: Time to diagnosis of type 2 diabetes mellitus.

Evaluate quality of life. Endpoint: Change in health and well-being (SF-12 questionnaire) from baseline at specified time points

Explore pharmacokinetics and immunogenicity in a subset of patients. Endpoint: ISIS 681257 drug exposure and anti-drug antibodies formation.

Evaluate effect of ISIS 681257 on aortic valve stenosis clinical endpoints. Endpoint: Time to the first occurrence of aortic valve procedure (percutaneous balloon aortic valvuloplasty, surgical (open-heart) aortic valve (AV) replacement, trans-femoral transcatheter AV replacement, transapical/trans-aortic AV replacement, valve-in-valve, other) or hospitalization for aortic valve stenosis.

### Study Design

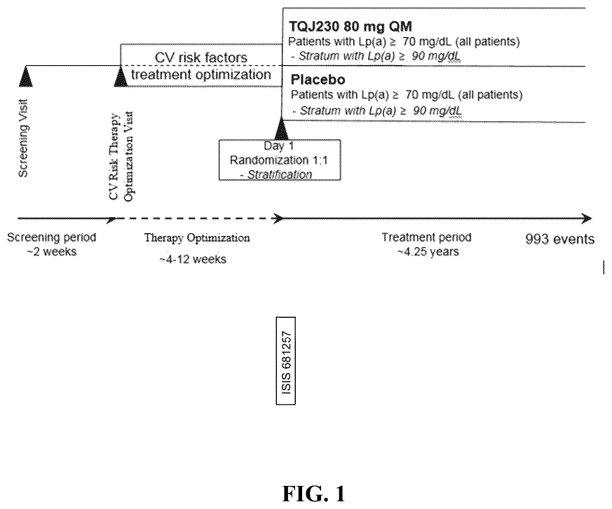

This is a randomized, double-blind, parallel group, placebo-controlled, multi-center study comparing ISIS 681257 80 mg s.c. QM to placebo in subjects with established CVD as evidenced by history of myocardial infarction, history of ischemic stroke or symptomatic peripheral artery disease (PAD) and elevated levels of Lp(a). Recruitment will target approximately 30% of randomized subjects to have had index myocardial infarction between ≥3 month and approximately 12 months prior to Randomization. The study consists of a screening period of approximately 2 weeks, followed by a period of CV risk factor therapy optimization of approximately 4-12 weeks, if required, and a double-blind treatment period. The minimum follow-up in double-blind period is required to be 2.5 years, the overall trial duration is expected to be approximately 4.25 years during which 993 primary endpoint events are expected to accumulate. The study will end when either 993 primary CV events have accumulated or all subjects have had at least 2.5 years of follow-up time-whatever comes later. (See FIG. 1)

Screening Period and Randomization after the Screening Period

At the study visit following the Screening visit, subject eligibility and the need for optimization of treatment of the CV risk factors (e.g., blood pressure, LDL-cholesterol and diabetes mellitus type 2) will be determined. Subjects who meet the eligibility criteria and do not require further optimization of their LDL-cholesterol treatment and/or of therapies for other CV risk factors according to the local practice/guidelines will be randomized, i.e. proceed directly to Day 1 visit.

### CV Risk Factor Optimization of Therapy (if Needed) and Randomization

Only subjects who require further optimization of their LDL-cholesterol (LDL-C) treatment and/or of their therapies for other CV risk factors according to the local practice/guidelines should complete additional visits during the “CV risk factor therapy optimization” period. At the CV Risk Factor Therapy Optimization visit they will be instructed to comply with lifestyle changes according to international recommendations, or equivalent local recommendations. Their treatment of CV risk factors will be optimized according to local practice/guidelines.

Subjects will come back to the site for a regular visit after approximately 4 weeks. Investigators will assess the efficacy and safety/tolerability of their optimized treatment. Treatment of the CV risk factors, or other treatments can be adjusted as needed.
Subjects who do not require further optimization of their treatment for CV risk factors according to the local practice/guidelines and meet the eligibility criteria can proceed to randomization (Day 1 visit)
If further optimization of CV risk factors treatment is considered necessary, the patient will complete additional visits

After approximately 4 weeks subjects will return for another study visit:
Subjects who do not require further optimization of their treatment for CV risk factors according to the local practice/guidelines and meet the eligibility criteria can proceed to randomization (Day 1 visit).
If further optimization of CV risk factors treatment is considered necessary, the period of the CV therapy optimization may be extended again for another 4 weeks, adding up to approximately 12 weeks of total therapy optimization. After completing these additional visits, eligible subjects will proceed to randomization (Day 1 Visit).

### Double-Blind Treatment Period

Eligible subjects will be randomized after screening or after the ‘CV risk factor treatment optimization’ period to subcutaneous injections of ISIS 681257 80 mg QM or placebo to be self-administered or administered by a caregiver or site personnel approximately every 30 days. Stratification based on the Lp(a) value at screening, ≥ or <90 mg/dL, and geographical region will be performed at randomization. Subjects will be followed according to the assessment schedule for efficacy, safety and other study-related assessments.

The following dose adjustments to the LDL-C lowering therapy can be done during the double-blind period:
Interruption, dose reduction or discontinuation of the LDL-C lowering treatment can be done in case of adverse events requiring such changes.
Dose increase or adding another LDL-C lowering treatment: Lipid assessments will be blinded after the randomization visit, however in case of an increase of LDL-C≥above a pre-specified algorithmic threshold from the randomization visit (or earlier, if this value is missing), investigators will be alerted by the Central laboratory to enable LDL therapy adjustment. This threshold algorithm will be provided and defined in the trial's lab manual. Investigators and site staff involved in the conduct of this trial and all medical personnel involved in the subject's care and management should refrain from obtaining lipid panels between the time from Randomization (Day 1) to study completion. If a lipid panel is obtained all reasonable actions must be taken to ensure the study subject is not informed of the results.

In addition, other risk factors for atherosclerotic disease such as high blood pressure and diabetes mellitus should be optimally treated during the double-blind period according to local practice/guidelines. Treatment dose for concomitant CV risk reducing medication and IL-6 inhibitors should be stable unless dose adjustment is required due to an adverse event.

### Population

Approximately 7,680 patients (male and female), aged 18 to 80 years, meeting the eligibility criteria will be randomized.

### Inclusion and Exclusion Criteria

### Key Inclusion Criteria:

Lp(a)≥70 mg/dL at the screening visit
Optimal LDL-cholesterol lowering treatment
Optimal treatment of other CV risk factors
Myocardial infarction: ≥3 months to ≤10 years prior to the screening visit, and/or
Ischemic stroke: ≥3 months to ≤10 years prior to the screening visit, and/or
Clinically significant symptomatic peripheral artery disease

### Key Exclusion Criteria:

Uncontrolled hypertension.
Heart failure New York Heart Association (NYHA) class IV
History of malignancy of any organ system
History of hemorrhagic stroke or other major bleeding
Platelet count <140,000 per μL
Active liver disease or hepatic dysfunction
Significant kidney disease
Pregnant or nursing women

### Detailed Inclusion Criteria

Subjects eligible for inclusion in this study must meet all of the following criteria:
1. Written informed consent must be obtained before any assessment is performed.
2. Male and female 18 to ≤80 years of age
3 Lp(a)≥70 mg/dL at the screening visit, measured at the Central laboratory
4. LDL-cholesterol lowering treatment at Randomization as follows:
   subjects must be on an optimal LDL-C lowering treatment to meet the target LDL-C level according to local practice/guidelines, or
   if subjects do not meet the target LDL-C level according to local practice/guidelines, they should be treated with the highest tolerated doses of statins and/or with other optimized LDL-lowering therapy (e.g. ezetimibe, cholesterol absorption inhibitor, fibrate, PCSK9 inhibitor), or
   if subjects have a contraindication or do not tolerate statin treatment, they must be treated with other optimized LDL-lowering therapy (e.g. ezetimibe, cholesterol absorption inhibitor, fibrate, PCSK9 inhibitor) according to local practice/guidelines
5. At the randomization visit subjects must be optimally treated for other CV risk factors according to local practice/guidelines
6. Established CV disease defined as ANY of the following three conditions:
   1) History of spontaneous myocardial infarction (not resulting from PCI or CABG) having occurred in the period ≥3 months to ≤10 years prior to the screening visit and documented as follows (Thygesen et al., “Fourth Universal Definition of Myocardial Infarction”, Circulation, p. e618-e651, 2018):
      Acute MI (hospitalization records): requires documentation of a rise and/or fall of cardiac biomarkers (preferably cardiac troponin) with at least one value above the 99th percentile of the upper reference limit (URL) and at least one of the following:
      Symptoms of ischemia
      ECG changes indicative of acute myocardial ischemia (new ST-T changes or new LBBB; please see Appendix 2 for ST and T-wave changes)
      Development of pathological Q waves (please see Appendix 2 for definitions of pathological Q waves)
      Imaging evidence of new loss of viable myocardium or new regional wall motion abnormality in a pattern consistent with an ischemic etiology
      Identification of a coronary thrombus by angiography including intracoronary imaging.
      Prior MI (no complete hospital records for acute event available): requires documentation of any one of the following:
         Pathological Q waves (please see Appendix 2 for definitions of pathological Q waves), with or without symptoms, in the absence of a non-ischemic cause
         Imaging evidence of a region of loss of viable myocardium that is thinned and fails to contract, in the absence of a non-ischemic cause
   2) History of ischemic stroke (an acute episode of focal cerebral, spinal, or visual dysfunction caused by infarction of central nervous system tissue) having occurred in the period ≥3 months to ≤10 years prior to the screening visit documented by CT scan, MRI or other visualization method. Transient ischemic attack or embolic stroke (not of atherosclerotic origin) are not qualifying events.
   3) Clinically significant symptomatic peripheral artery disease, evidenced by intermittent claudication with an ankle-brachial index≤0.90 and/or limb amputation or re-vascularization due to lower limb ischemia. Thromboangiitis obliterans is not a qualifying event.

### Detailed Exclusion Criteria

Subjects meeting any of the following criteria are not eligible for inclusion in this study.
1. Uncontrolled hypertension defined as sitting systolic blood pressure (SBP) ≥160 mmHg and/or diastolic blood pressure (DBP) ≥100 mmHg (mean of 3 measurements for each SBP and DBP assessment) at the Screening visit.
2. Treatment with niacin in the 3 months before the screening visit; niacin in multi-vitamins is allowed
3. Treatment with stable dose of a PCSK9 inhibitor (evolocumab, alirocumab) for less than 12 weeks before Randomization
4 Treatment with lipoprotein apheresis, or already planned to start lipoprotein apheresis during the study
5. Within 3 months of screening and between Screening visit and Randomization visit (Day 1): myocardial infarction, stroke, coronary or lower limb re-vascularization, major cardiac or non-cardiac surgery. The subjects can be re-screened 3 months after the relevant event/procedure.
6. Planned or expected cardiac, cerebrovascular or peripheral artery surgery or coronary re-vascularization after Randomization visit (Day 1)
7. Heart failure New York Heart Association (NYHA) class IV at Screening visit or at Randomization visit (Day 1)
8. History of hemorrhagic stroke or other major bleeding, or if occurring between Screening visit and Randomization visit
9. Severe concomitant non-CV disease that is expected to reduce life expectancy to less than 5 years, at Screening visit or at Randomization visit (Day 1)
10. Known active severe infection or major hematologic, metabolic, gastrointestinal or endocrine dysfunction (e.g. uncontrolled thyroid dysfunction or uncontrolled diabetes mellitus) in the judgment of the investigator, at Screening visit or at Randomization visit (Day 1)
11. History of malignancy of any organ system (other than localized basal cell carcinoma or squamous cell carcinoma of the skin, or in situ cervical cancer), treated or untreated, within the past 5 years, or if diagnosed between Screening visit and Randomization visit (Day 1), regardless of whether there is evidence of local recurrence or metastases.
12. Platelet count <140,000 per μL from central laboratory test at Visit 1, confirmed by a second central laboratory test prior to the Randomization visit (Day 1)
13. eGFR ≤30 mL/min/1.73 m² from central laboratory test at Visit 1, confirmed by a second central laboratory test prior to the Randomization visit (Day 1); or patient on dialysis
14. Significant glomerular disease (including but not limited to IgA nephropathy, diabetic nephropathy, systemic lupus erythematosus, etc.) with urinary protein-creatinine ratio (PCR)>500 mg/g (50 mg/mmol) at Visit 1, confirmed by a second central laboratory test prior to the Randomization visit (Day 1)
15. Active liver disease or hepatic dysfunction, defined as AST or ALT ≥2 times the ULN from central laboratory test at Screening visit, confirmed by a second central laboratory test prior to the Randomization visit (Day 1)
16. Total bilirubin ≥1.5 times the ULN from central laboratory test at Screening visit, confirmed by a second central laboratory test prior to the Randomization visit (Day 1)
17. Positive HIV, Hepatitis C screening or Hepatitis B Surface Antigen tests from central laboratory test at Screening visit
18. Any other conditions, at Screening visit or between Screening visit and Randomization visit (Day 1), which in the opinion of the Investigator would make the patient unsuitable for inclusion, or could interfere with the patient participating in or completing the study
19. Treatment with an oligonucleotide or SiRNA within 9 months of screening visit. Exceptions are GalNac oligonucleotide or GalNac siRNA which can be used if approved by health authorities and if allowed for use in the study by the Sponsor.
20. History of hypersensitivity to the study drug or their excipients or to drugs of similar chemical classes
21. Use of other investigational drugs within 5 half-lives of Screening visit, or within 30 days, whichever is longer
22. Unwillingness or inability (e.g. physical or cognitive) to comply with study procedures and medication administration (injections) and schedule
23. Pregnant or nursing (lactating) women
24. Women of child-bearing potential, defined as all women physiologically capable of becoming pregnant, unless they are using highly effective methods of contraception during dosing of investigational drug. Such methods include:
   Total abstinence (when this is in line with the preferred and usual lifestyle of the subject. Periodic abstinence (e.g., calendar, ovulation, symptothermal, post-ovulation methods) and withdrawal are not acceptable methods of contraception.
   Female sterilization (have had surgical bilateral oophorectomy with or without hysterectomy), total hysterectomy, or bilateral tubal ligation at least six weeks before taking study treatment. In case of oophorectomy alone, only when the reproductive status of the woman has been confirmed by follow up hormone level assessment
   Male sterilization (at least 6 months prior to screening). For female subjects on the study, the vasectomized male partner should be the sole partner for that subject
   Use of oral, (estrogen and progesterone), injected or implanted hormonal methods of contraception or placement of an intrauterine device (IUD) or intrauterine system (IUS), or other forms of hormonal contraception that have comparable efficacy (failure rate <1%), for example hormone vaginal ring or transdermal hormone contraception

In case of use of oral contraception women should have been stable on the same pill for a minimum of 3 months before taking study treatment.

Women are considered post-menopausal and not of child bearing potential if they have had 12 months of natural (spontaneous) amenorrhea with an appropriate clinical profile (e.g. age, appropriate history of vasomotor symptoms) or have had surgical bilateral oophorectomy (with or without hysterectomy), total hysterectomy or bilateral tubal ligation at least six weeks ago. In the case of oophorectomy alone, only when the reproductive status of the woman has been confirmed by follow up hormone level assessment is she considered not of child-bearing potential.

If local regulations deviate from the contraception methods listed above to prevent pregnancy, local regulations apply and will be described in the Informed consent form (ICF).

### Treatment

Subjects will be randomized 1:1 to administer sub-cutaneous injections of compound ISIS 681257 80 mg s.c. QM or placebo. Injections will be self-injected or administered by a caregiver subcutaneously once a month with the injection device compound ISIS 681257 Needle Safety Device (NSD). Details on how injections should be performed are found herein below. Instructions for prescribing and taking the study treatment.

The investigational and control drugs used in the study are shown in Table 1 below:

TABLE 1
Investigational and control drugs

| Investigational/ | Pharmaceutical | Route of | | Sponsor (global |
|---|---|---|---|---|
| Control Drug | Dosage Form | Administration | Supply type | or local) |
| ISIS 681257 | Solution for | Subcutaneous | Solution for | Sponsor global |
| 80 mg | injection | injection | injection in pre- | |
| | | | filled syringe | |
| Placebo | Solution for | Subcutaneous | Solution for | Sponsor global |
| | injection | injection | injection in pre- | |
| | | | filled syringe | |

### Treatment Duration

The study duration is expected to be approximately 4.25 years with a minimum follow-up time of approximately 2.5 years (unless death or withdrawal of consent); during this period, the required number of confirmed primary endpoint events is expected to be accumulated. The study will complete when either 993 primary CV events have accumulated or all subjects have had at least 2.5 years of follow-up time-whatever comes later. Subjects may be discontinued from the study drug for safety reasons and/or at the discretion of the investigator or the subject. They will continue to be followed up in the study unless they withdraw their informed consent.

### Other Treatment(s)

All medications, procedures and significant non-drug therapies used by the subject in the 3 months prior to randomization must be recorded in the prior/concomitant medications/significant non-drug therapies or procedures electronic case report form (eCRF) pages independent if these will be continued during the study or not. Each concomitant drug must be individually assessed against all exclusion criteria.

All medications, procedures and significant non-drug therapies (including physical therapy, lifestyle instructions and blood transfusions) administered after the subject was enrolled into the study must also be recorded in the corresponding eCRF pages. New concomitant drug must be assessed against prohibited medication prior to its initiation. Doses of lipid-lowering and anti-platelet drugs should be recorded throughout the entire duration of the study.

If in doubt, the investigator should contact the Novartis medical monitor before randomizing a subject or allowing a new medication to be started. If the subject is already enrolled, contact Novartis to determine if the subject should continue study medication.

It is recommended that subjects participating in the trial or their caregivers inform healthcare professionals not involved in the study for the potential risk of thrombocytopenia, so that any concomitant therapy which may result in thrombocytopenia and/or bleeding (e.g., heparin, oral anti-coagulants, direct thrombin inhibitors, Factor Xa inhibitors, and niacin) is managed appropriately and subject should be closely monitored. A subject card with information about the potential risk of bleeding will be provided to subjects at the Randomization visit (Day 1).

### Prohibited Medication

Use of the treatments displayed in Table 2 below are not allowed.

TABLE 2
Prohibited medication and procedures

| Medication | Prohibition period | Action taken |
|---|---|---|
| Oligonucleotide other than ISIS | Full Trial duration | Discontinue study treatment |
| 681257 or SiRNA treatment* | | |
| Lipoprotein apheresis** | Full Trial duration | Discontinue study treatment |
| Niacin treatment** | Full Trial duration | Discontinue study treatment |
| Other investigational treatments | Full Trial duration | Discontinue study treatment |

*Exceptions are GalNac oligonucleotide or GalNac siRNA which can be used if approved by health authorities and if allowed for use in the study by the Sponsor
**If during the study lipoprotein apheresis becomes indicated according to local practice, study drug must be discontinued and patient will continue to be followed up in the study
***Niacin in multi-vitamins is allowed

### Dose Escalation and Dose Modification

Dose escalation and dose modification-Investigational treatment interruptions are not permitted unless for safety reasons.

### Screening

Screening activities must be initiated only after the patient has signed the Informed Consent Form (ICF). Re-screening is allowed once up to 6 months after the screening visit (Visit 1).

In the case where a safety laboratory assessment at screening outside of the range specified in the exclusion criteria, the assessment may be repeated once prior as required by the exclusion criteria. If the repeat value remains outside of the specified ranges, the subject is not eligible for the study.

### Subject Demographics Other Baseline Characteristics

Patient demographic and baseline characteristic data to be collected on all subjects include: age, sex, race, ethnicity, source of patient referral, relevant family and individual medical history/current medical condition present before signing informed consent (where possible, diagnoses and not symptoms will be recorded), as well as relevant laboratory tests at screening.

Investigators will have the discretion to record abnormal test findings on the medical history eCRF whenever in their judgment, the test abnormality occurred prior to the informed consent signature.

### Efficacy

Efficacy is assessed by clinical endpoint committee adjudicated CV death, non-fatal myocardial infarction, non-fatal stroke, urgent coronary revascularization requiring hospitalization.

The efficacy assessments are specified below with the assessment schedule detailing when each assessment is to be performed.
Efficacy assessment 1—An independent CEC that is blinded to treatment assignments will review and adjudicate all clinical events that constitute the primary and secondary composite endpoints.
Efficacy assessment 2—Changes from baseline at study endpoint in the efficacy biomarkers Lp(a), hsCRP and ApoB, as well as in total cholesterol, LDL-cholesterol, non-HDL-cholesterol, HDL-cholesterol, and triglycerides will be assessed. After randomization (Day 1), the results from Lp(a) and all lipids (total cholesterol, LDL-cholesterol, non-HDL-cholesterol, HDL-cholesterol and triglycerides) must be blinded to subjects, investigator staff, persons performing the assessments, and the CTT.

In case of an increase of LDL-C above a pre-specified algorithmic threshold from the randomization value, or if the latter is missing, from the latest value prior to randomization measured at the Central laboratory, investigators will be alerted by the Central laboratory. In addition, investigators and site staff involved in the conduct of this trial and all medical personnel involved in the subject's care and management should refrain from obtaining lipid panels between the time from Randomization (Day 1) to study completion. If a lipid panel is obtained all reasonable actions must be taken to ensure the study subject is not informed of the results.

### Safety

Key safety assessment includes adverse event monitoring, physical examination, laboratory assessments, and ECG measurement.

A complete physical examination will be performed at the screening visit (Visit 1); at the consequent visits only a short physical exam will be performed; and at remaining study site visits only a brief physical exam will be performed.

### Additional Assessments

### Clinical Outcome Assessments (COAs)

Health status and well-being of subjects enrolled in the double-blind treatment period will be assessed with the SF-12 questionnaire, version 2 (SF-12 v2). SF-12 v2 covers the same eight health domains as the SF-36 with substantially fewer questions, making it a more practical research tool in patients with chronic conditions.

The absolute values and changes from baseline for the domain-specific scores and the two summary scores will be descriptively summarized by visit and treatment. Between-treatment analysis based on the statistical modeling for changes from baseline at post-BL visits in the two summary scores will be provided as well.

The above analyses will be performed in FAS patients whose SF-12 data are collected.

### Biomarkers

Blood samples for assessment of Lp(a) will be collected at certain study visits. Other blood biomarkers used for efficacy assessments are hsCRP and apoB.

### Data Analysis

The primary efficacy variable (time-to-the-first occurrence of the primary endpoint events) will be analyzed using a Cox proportional hazards model with treatment, region, subpopulation indicator (baseline Lp(a) level <, or ≥90 mg/dL) and an interaction term of treatment by subpopulation indicator as factors. The two primary endpoint hypotheses will be tested using a weighted Dunnett test, following the principles in Glimm et al.

Secondary endpoints will be analyzed using the same Cox-regression model as for the primary analysis model.

The estimated hazards ratios and the corresponding two-sided confidence intervals and the p-values in the full and subpopulations from these Cox regression models will be provided separately.

The primary endpoint and secondary endpoints of the MACE composite (CV death, non-fatal MI and non-fatal stroke) and the coronary composite (death due to CHD events, non-fatal MI and urgent coronary revascularization requiring hospitalization) in the full and subpopulation will be included in a closed multiple testing procedure, in order to control the type I error.

The overall study type I error is controlled at 2.5% (one-sided). The one-sided alpha level used for the final analysis will be 2.45%, after accounting for the alpha used in the two interim efficacy analyses.

The hypotheses for all-cause mortality in the full and subpopulation will be tested at the full alpha (one-sided 2.5%) separately. Efficacy analyses will be performed in the full analysis set (FAS).

### Details:

### Analysis of the Primary Endpoint(s)

The primary aim of the study is to demonstrate the superiority of ISIS 681257 compared to placebo in reducing the risk of the expanded major adverse cardiovascular events (cardiovascular death, non-fatal MI, non-fatal stroke and urgent coronary re-vascularization requiring hospitalization). Unless otherwise specified, all time-to-event analyses will be based on adjudicated events occurring during the double-blind treatment period of the study.

The primary patient populations to whom the proposed analyses apply are all subjects in the FAS (full population), and subjects in the FAS whose baseline Lp(a) levels ≥90 mg/dL (subpopulation). The primary endpoint defined below will consider intercurrent events corresponding to discontinuation from the study or deaths from non-CV causes as random, estimating the cause-specific hazard ratio. The estimation will use the follow up data available up to the occurrence of the events of interest or censoring time otherwise, regardless of adherence to study medication. The analysis set on which the analyses will be based is the Full Analysis Set (FAS).

In this patient population, the estimand targeted concerns the reduction of the rate of primary MACE in subjects with established CV disease as defined in the inclusion criteria and Lp(a)≥70 mg/dL (full study population) and/or Lp(a)≥90 mg/dL (subpopulation of interest), including the effect of region. Below the endpoint of interest and the proposed evaluation of treatment benefit in the full population and the subpopulation of interest supporting the proposed estimand are detailed.

### Definition of Primary Endpoint(s)

The primary efficacy variable is the time to first occurrence of an expanded major adverse cardiovascular event (MACE), which is a composite endpoint consisting of cardiovascular death, non-fatal MI, non-fatal stroke and urgent coronary re-vascularization requiring hospitalization. An independent CEC will review and adjudicate the clinical events that constitute the composite of the primary endpoints on a blinded basis.

The time-to-event is computed as the number of days from randomization to the onset of the primary endpoint event. Data on subjects who do not reach the primary endpoint by the study end date will be censored at the latest date they are known to be at risk in the study.

### Statistical Model, Hypothesis, and Method of Analysis

The two primary statistical null hypotheses to be tested are:

$${H}_{10} : {λ}_{12} / {λ}_{11} \geq 1 versus {H}_{1 a} : {λ}_{12} / {λ}_{11} < 1 \qquad ( 1 )$$

$${H}_{20} : {λ}_{22} / {λ}_{21} \geq 1 versus {H}_{2 a} : {λ}_{22} / {λ}_{21} < 1 \qquad ( 2 )$$

where λ1₁, λ₁₂ and λ₂₁, λ₂₂ are hazards of first CEC confirmed expanded MACE for ISIS 681257 group and placebo group in the full population and the subpopulation, respectively.

The primary efficacy variable will be analyzed using Cox's proportional hazards model with treatment, region, subpopulation indicator (baseline Lp(a) level <, or ≥90 mg/dL) and an interaction term of treatment by subpopulation indicator as factors. A model-based Dunnett's test approach as described in Glimm et al. (Glimm et al., “An approach to confirmatory testing of subpopulations in clinical trials, Biom J., p. 897-913, 2015) will be used to test the two primary hypotheses. The above Cox's proportional hazards model can be expressed as:

$$λ ( t ) = {λ}_{0} ( t ) * exp ( {β}_{1} * {x}_{1} + {β}_{2} * {x}_{2} + {β}_{3} * {x}_{3} + {β}_{4} * {x}_{1} * {x}_{3} ) \qquad ( 3 )$$

where λ(t) is the hazard at time t and Δ0(t) is the baseline hazard, xi (i=1, 2, 3) are the covariates. More specifically, x1 is the treatment group indicator, x2 is the region indicator, x3 is the indicator for subpopulation indicator (Lp(a)< or ≥90 mg/dL) and x1*x3 is the interaction term of treatment by subpopulation indicator. The model can be parameterized so that β1+β4 is the log hazard ratio of ISIS 681257 versus placebo in the subpopulation and β1+w β4 is the log hazard ratio of ISIS 681257 versus placebo for the full population, where w is the proportion of patients in the subpopulation (Lp(a)≥90 mg/dL).

Under model (3), the two primary hypotheses (1) and (2) can be expressed equivalently as:

$${H}_{10} : {β}_{1} + w {β}_{4} \geq 0 versus {H}_{1 a} : {β}_{1} + w {β}_{4} < 0 \qquad ( 4 )$$

$${H}_{20} : {β}_{1} + {β}_{4} \geq 0 versus {H}_{2 a} : {β}_{1} + {β}_{4} < 0 \qquad ( 5 )$$

As shown in Glimm et al. (2015), the two test statistics derived from the model (3) for the two hypotheses (4) and (5) follow a bi-variate Normal distribution asymptotically. Based on these two test statistics, Dunnett's test critical values and adjusted 1-sided p-values for the two primary hypotheses will be calculated. The overall study type I error will be controlled at 2.5% (one-sided). The one-sided significance level of a to be used for the final analysis will be 2.45%, after accounting for the alpha used in the interim efficacy analyses according to an interim analyses plan.

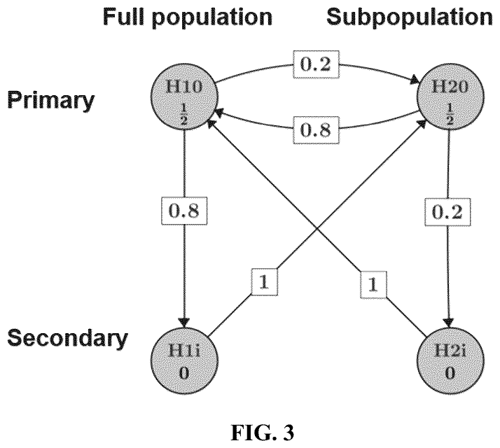

The testing procedure is graphically presented in FIG. 3, and outlined in the following steps:
First, a weighted Dunnett test with equal weights assigned to each of the two primary hypotheses (H10 and H20) is performed. This test exploits the correlation of the primary endpoint between the full and the subpopulation, with the test statistic derived from the primary analysis model as described above
If one primary hypothesis is rejected, a fraction of its significance level will be passed to the other primary hypothesis while the remaining alpha will be propagated to the family of secondary endpoints in the same population, according to pre-specified weights as indicated in the graph
Within a family of secondary endpoints (H1i or H2i), the weighted Simes testing procedure will be used.
If both secondary hypotheses in overall population are rejected, the alpha is propagated to the primary endpoint in the subpopulation; vice versa for the alpha passing from H2i to H10. (See FIG. 3)

The nodes for secondary (H1i or H2i) represent families of null hypotheses related to the first two secondary endpoints in full and subpopulations. The secondary endpoint of all-cause mortality, in the full population and the subpopulation, will not be included in the multiple testing procedure.

If at least one of the two primary hypotheses is rejected under the above testing procedure the study can be claimed a success.

The estimated hazards ratios and the corresponding unadjusted two-sided confidence intervals in the full and subpopulations from the Cox regression model (3) will be provided separately. The FAS will be used for the primary analysis.

### Analysis of Secondary Endpoints

### Secondary Endpoints

There are three secondary endpoints defined, and six secondary hypotheses to be tested for these three secondary endpoints in the full and subpopulation respectively:
Time to the first occurrence of the CEC confirmed composite endpoint of MACE: CV death, non-fatal MI, and non-fatal stroke—H11 and H21 are the relevant secondary hypotheses in the full and subpopulation
Time to the first occurrence of the CEC confirmed composite endpoint of CHD outcomes: CHD death, non-fatal MI, urgent coronary re-vascularization requiring hospitalization—H12 and H22 are the relevant secondary hypotheses in the full and subpopulation
Time to all-cause death from randomization to the end of study—H13 and H23 are the relevant secondary hypotheses in the full and subpopulation.

All secondary endpoints will be analyzed in the FAS using the same Cox-regression model as for the primary analysis model described above. The estimated hazards ratios and the corresponding two-sided confidence intervals and the p-values in the full and subpopulations from these Cox regression models will be provided separately.

As stated above, the two families of secondary hypotheses H1i (including H11 and H12) and H2i (including H21 and H22) are included in the multiple testing procedure as presented in FIG. 3. A weighted Simes testing method will be used to test the two secondary hypotheses within each family (H1i or H2i) separately in the following steps:
First, the two nominal p-values from the above Cox-regression models for the two secondary endpoints in a family (e.g. p11 for MACE, and p12 for coronary composite endpoint in the full population) are compared to the alpha passed to this family as specified in FIG. 3. If both nominal p-values <=this local alpha, both secondary hypotheses within this family are rejected, otherwise continue
Comparing the individual nominal p-values to weighted local alpha respectively with pre-specified weights 0.8 and 0.2. If the p-value for the MACE is ≤80% of the local alpha, the null hypothesis for the MACE is rejected; if the p-value for the coronary composite endpoint is ≤20% of the local alpha, the null hypothesis for the coronary composite endpoint is rejected.

The hypotheses corresponding to all-cause mortality in the full and subpopulation (H13 and H23) are not included in the multiple testing procedure, and will be tested at full alpha (1-sided 2.5%) separately. Kaplan-Meier plots by treatment will be provided for each secondary endpoint in the full and subpopulation separately. The components of the composite secondary endpoints will also be analyzed individually, using the same Cox-regression model as for the primary analysis. The frequency and percentage of patients who reach the secondary composite endpoints will be provided by treatment group in the FAS. The composite secondary endpoints and their components based on investigator-reported events will be similarly analyzed and presented.

### Sample Size Calculation

### Primary Endpoint(s)

The sample size estimation below is based on a 1:1 randomization between ISIS 681257 and placebo, and a one-sided significance level of 0.0245, after adjusting for the efficacy interim analyses planned. The calculations were performed in two steps. First, the calculation was done for the MACE primary endpoint using a conventional log-rank test at one-sided significance level of 0.01225 for the primary hypothesis in the full-population (assuming a more conservative Bonferroni alpha split between two primary hypotheses).

It is assumed that there is a delayed treatment effect following a piecewise exponential distribution with a hazard ratio of 0.90 during the first 12 months after randomization and then a constant hazard ratio of 0.764 after 12 months in favor of the ISIS 681257 group. Under this assumption, 993 primary endpoint MACE events (i.e. 993 subjects with MACE events during the double-blind period) will provide approximately 88% power at one-sided alpha level of 0.01225.

Assuming an annualized primary endpoint MACE event rate of 4.6% in the placebo group, an enrollment period of 1.5 years, a maximum follow-up of 4.25 years and a cumulative censoring rate of 10% for primary endpoint events due to loss to follow-up or non-cardiovascular deaths, a total sample size of 7,680 subjects will be required to obtain 993 primary endpoint MACE events.

The assumption of 4.6% annual event rate for the primary composite endpoint event was based on data from the FOURIER trial (Sabatine et al., “Evolocumab and Clinical Outcomes in Patients with Cardiovascular Disease”, N. Engl. J. Med., p. 1713-1722, 2017). The assumption of hazard ratio of 0.90 during the first 12 months after randomization and then a constant hazard ratio of 0.764 after 12 months corresponds to a hazard ratio of 0.805 over the entire double-blind period (i.e. 19.5% hazard rate reduction of ISIS 681257 versus placebo), given the enrollment period of 1.5 years and maximum follow-up of 4.25 years.

Secondly, power was assessed for the overall study and the two primary hypotheses using the testing procedure described herein above. Table 3 presents the power of the study at the one-sided alpha of 2.45% given the sample size of 7,680 and number of primary endpoint events 993 as calculated in step 1 above, and the various scenarios of the underlying true effect size of TQJ230 versus placebo as assumed in the first two columns.

TABLE 3
Power calculations given target event number and sample
size under assumptions on treatment effect size

| | HRR | HRR | Power | Power | Power |
|---|---|---|---|---|---|
| | (Full | (subpop- | (Overall | (Full | (subpop- |
| | Population) | ulation) | study) | population) | ulation) |
| Case | (%) | (%) | (%) | (%) | (%) |
| 1 | 0.15 | 0.20 | 0.76 | 0.64 | 0.64 |
| 2 | 0.20 | 0.25 | 0.95 | 0.90 | 0.86 |
| 3 | 0.20 | 0.20 | 0.92 | 0.90 | 0.65 |
| 4 | 0.18 | 0.18 | 0.84 | 0.82 | 0.54 |

The annualized primary endpoint event rate in placebo group was assumed to be 4.6% in the full population; 5.06% in subjects with Lp(a)≥90 mg/dL) and 4.14% in subjects with Lp(a)<90 mg/dL.

Calculations were made using the software package R (version 3.4.3).

In order to achieve the required number of events in an acceptable time frame and preserve the target power of the study, the event rate of the primary endpoint will be monitored in a blinded fashion so that adjustments can be made to the number of subjects to be randomized and/or the duration of follow-up as needed. Given the intention in the study protocol of ensuring a minimum 2.5 years of follow-up for the last randomized subjects, it is foreseen that the number of primary endpoint events accrued in the study could be larger than the required number of events derived in the sample size calculations.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1-60. (canceled)

61. A method of treating a patient, the method comprising administering to the patient a unit dose comprising from about 75 mg to about 85 mg of a compound or pharmaceutically acceptable salt thereof by subcutaneous injection once a month, wherein the compound has the following structure: 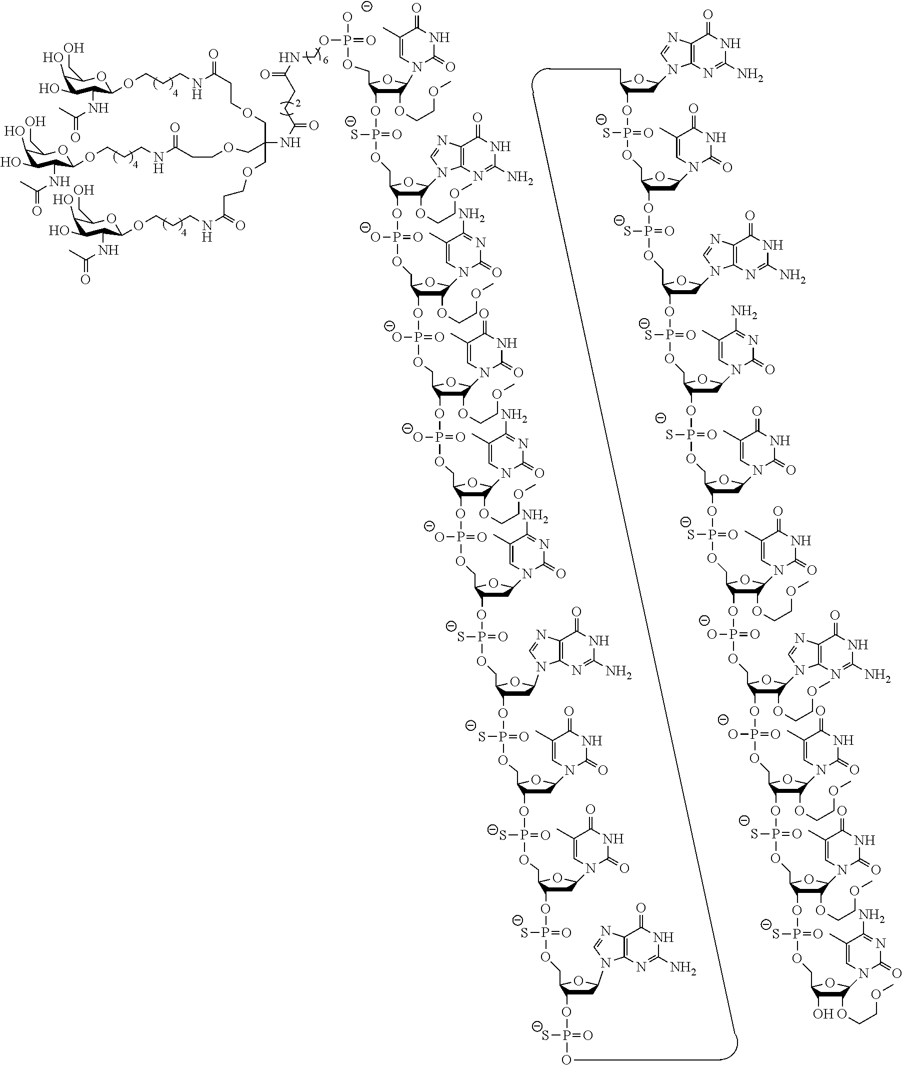
and wherein the patient has established cardiovascular disease and elevated Lp(a) levels, to thereby reduce the risk of cardiovascular death, myocardial infarction, stroke, or urgent coronary re-vascularization.

62. The method according to claim 61, wherein the unit dose comprises about 80 mg of the compound.

63. The method according to claim 61, wherein the unit dose comprises 80 mg of the compound.

64. The method according to claim 61, wherein the patient has elevated Lp(a) levels ≥70 mg/dL prior to the first administration of the compound.

65. The method according to claim 61, wherein the compound is in the form of a sodium salt.

66. The method according to claim 65, wherein the compound has the following structure: [chemical structure]
